SEQUENCE LISTING (53 sequences)

SEQ ID NO: 1 - RNA, 71 nt, Artificial Sequence
  hsa-mir-1-5p
ugggaaacauacuucuuuauaugcccauauggaccugcuaagcuauggaauguaaagaaguauguaucuca

SEQ ID NO: 2 - RNA, 71 nt, Artificial Sequence
  hsa-mir-208a-5p
ugacgggcgagcuuuuggcccggguuauaccugaugcucacguauaagacgagcaaaaagcuuguugguca

SEQ ID NO: 3 - RNA, 77 nt, Artificial Sequence
  hsa-mir-208b-5p
ccucucagggaagcuuuuugcucgaauuauguuucugauccgaauauaagacgaacaaaagguuugucugagggcag

SEQ ID NO: 4 - RNA, 85 nt, Artificial Sequence
  hsa-mir-122
ccuuagcagagcuguggagugugacaaugguguuugugucuaaacuaucaaacgccauuaucacacuaaauagcuacugcuaggc

SEQ ID NO: 5 - RNA, 88 nt, Artificial Sequence
  hsa-mir-133a-1
acaaugcuuugcuagagcugguaaaauggaaccaaaucgccucuucaauggauuugguccccuucaaccagcuguagcuaugcauuga

SEQ ID NO: 6 - RNA, 83 nt, Artificial Sequence
  hsa-mir-488-5p
gagaaucaucucucccagauaauggcacucucaaacaaguuuccaaauuguuugaaaggcuauuucuuggucagaugacucuc

SEQ ID NO: 7 - DNA, 22 bp, Artificial Sequence
  target segment of hsa-mir-1-5p
atgggcatataaagaagtatgt

SEQ ID NO: 8 - DNA, 22 bp, Artificial Sequence
  target segment of hsa-mir-208a-5p
gtataacccgggccaaaagctc

SEQ ID NO: 9 - DNA, 21 bp, Artificial Sequence
  target segment of hsa-mir-208b-5p
acataattcgagcaaaaagct

SEQ ID NO: 10 - DNA, 22 bp, Artificial Sequence
  target segment of hsa-mir-122
caaacaccattgtcacactcca

SEQ ID NO: 11 - DNA, 22 bp, Artificial Sequence
  target segment of hsa-mir-133a-1
cagctggttgaaggggaccaaa

SEQ ID NO: 12 - DNA, 21 bp, Artificial Sequence
  target segment of hsa-mir-488-5p
ttgagagtgccattatctggg

SEQ ID NO: 13 - DNA, 6 bp, Artificial Sequence
  EXG-Link01
cttgac

SEQ ID NO: 14 - DNA, 6 bp, Artificial Sequence
  EXG-Link02
ccatag

SEQ ID NO: 15 - DNA, 6 bp, Artificial Sequence
  EXG-Link03
tttcta

SEQ ID NO: 16 - DNA, 6 bp, Artificial Sequence
  EXG-Link04
caagct

SEQ ID NO: 17 - DNA, 6 bp, Artificial Sequence
  EXG-Link05
gatcta

SEQ ID NO: 18 - DNA, 486 bp, Artificial Sequence
  miRNA Sponge Region-1x hsa-mir-1; 2x hsa-mir- 208a; 3x hsa-mir-208b; 3x hsa-mir-122 (as in EXG202)
ttgaatgaggcttcagtactttacagaatcgttgcctgcacatcttggaaacacttgctgggattacttcttcaggttaacccaacagaaggctcgagaaggtatattgctgttgacagtgagcgctacatacttctttatatgcccatgtgaagccacagatgatgggcatataaagaagtatgtattgcctactgcctcggaattcaaggggctactttaggagcaattatcttgtttactaaaactgaataccttgctatctctttgatacatttttacaaagctgaattaaaatggtataaattaaatcacttttttctagtataacccgggccaaaagctcagtataacccgggccaaaagctcgacataattcgagcaaaaagctaacataattcgagcaaaaagctcttgacaaacaccattgtcacactccaacaaacaccattgtcacactccaacaaacaccattgtcacactcca

SEQ ID NO: 19 - DNA, 237 bp, Artificial Sequence
  miRNA Sponge Region-2x hsa-mir-208a; 2x hsa- mir-208b; 3x hsa-mir-122; 3x hsa-mir-133a (as in EXG204)
gtataacccgggccaaaagctcagtataacccgggccaaaagctcgacataattcgagcaaaaagctaacataattcgagcaaaaagctcttgacaaacaccattgtcacactccaacaaacaccattgtcacactccaacaaacaccattgtcacactccaccatagacagctggttgaaggggaccaaaacagctggttgaaggggaccaaaacagctggttgaaggggaccaaa

SEQ ID NO: 20 - DNA, 258 bp, Artificial Sequence
  miRNA Sponge Region-2x hsa-mir-208a; 2x hsa- mir-208b; 3x hsa-mir-122; 2x hsa-mir-488; 2x hsa-mir-1 (as in EXG205)
gtataacccgggccaaaagctcagtataacccgggccaaaagctcgacataattcgagcaaaaagctaacataattcgagcaaaaagctcttgacaaacaccattgtcacactccaacaaacaccattgtcacactccaacaaacaccattgtcacactccaccatagattgagagtgccattatctgggattgagagtgccattatctgggaatgggcatataaagaagtatgtaatgggcatataaagaagtatgt

SEQ ID NO: 21 - DNA, 486 bp, Artificial Sequence
  miRNA Sponge Region-1x hsa-mir-133a; 2x hsa-mir-208a; 2x hsa-mir-208b; 3x hsa-mir-122 (as in EXG206)
ttgaatgaggcttcagtactttacagaatcgttgcctgcacatcttggaaacacttgctgggattacttcttcaggttaacccaacagaaggctcgagaaggtatattgctgttgacagtgagcgcttttggtccccttcaaccagctggtgaagccacagatgcagctggttgaaggggaccaaaattgcctactgcctcggaattcaaggggctactttaggagcaattatcttgtttactaaaactgaataccttgctatctctttgatacatttttacaaagctgaattaaaatggtataaattaaatcacttttttctagtataacccgggccaaaagctcagtataacccgggccaaaagctcgacataattcgagcaaaaagctaacataattcgagcaaaaagctcttgacaaacaccattgtcacactccaacaaacaccattgtcacactccaacaaacaccattgtcacactcca

SEQ ID NO: 22 - DNA, 2596 bp, Artificial Sequence
  EXG204-LmiR122-HmiR133
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggaattcccgggatatcgtcgacccacgcgtccgggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctagtataacccgggccaaaagctcagtataacccgggccaaaagctcgacataattcgagcaaaaagctaacataattcgagcaaaaagctcttgacaaacaccattgtcacactccaacaaacaccattgtcacactccaacaaacaccattgtcacactccaccatagacagctggttgaaggggaccaaaacagctggttgaaggggaccaaaacagctggttgaaggggaccaaacaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 23 - DNA, 2595 bp, Artificial Sequence
  EXG207-LmiR122-HmiR133
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctatggccatgagcagcggaggaagcggaggaggagtgcccgagcaagaggacagcgtgctgtttaggagaggaaccggacagagcgatgactccgatatctgggacgacaccgctctgatcaaggcctatgacaaagccgtggcctccttcaagcacgctctgaagaatggcgatatctgtgagacctccggcaaacctaagaccacccccaagaggaagcccgccaagaagaacaagtcccagaagaagaataccgccgctagcctccagcagtggaaagtgggcgataagtgcagcgccatttggagcgaggatggatgcatctaccccgccaccattgccagcatcgacttcaagagggagacatgcgtggtggtgtataccggatacggaaatagagaggagcagaatctgagcgatctgctgtcccccatctgcgaggtggccaataatatcgagcagaacgcccaagagaacgagaacgaaagccaagtgtccaccgatgagagcgagaactccagaagccccggaaacaagtccgacaacatcaaacccaagagcgccccttggaacagctttctgcctcctcccccccccatgcccggccctagactgggacccggcaagcccggactgaagttcaacggacccccccctcctcctcccccccctcctcctcatctgctgagctgctggctcccccctttccctagcggcccccccattatccccccccctccccctatctgtcccgacagcctcgatgacgctgacgccctcggaagcatgctgatcagctggtacatgagcggctaccacaccggatactacatgggcttcagacagaaccagaaggagggcagatgctcccactctctgaactgaggagaaatgctggcatagagcagcactaaatgacaccactaaagaaacgatcagacagatctagtataacccgggccaaaagctcagtataacccgggccaaaagctcgacataattcgagcaaaaagctaacataattcgagcaaaaagctcttgacaaacaccattgtcacactccaacaaacaccattgtcacactccaacaaacaccattgtcacactccaccatagacagctggttgaaggggaccaaaacagctggttgaaggggaccaaaacagctggttgaaggggaccaaacaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 24 - DNA, 2595 bp, Artificial Sequence
  EXG209-LmiR122-HmiR133
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctagtataacccgggccaaaagctcagtataacccgggccaaaagctcgacataattcgagcaaaaagctaacataattcgagcaaaaagctcttgacaaacaccattgtcacactccaacaaacaccattgtcacactccaacaaacaccattgtcacactccaccatagacagctggttgaaggggaccaaaacagctggttgaaggggaccaaaacagctggttgaaggggaccaaacaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 25 - DNA, 2594 bp, Artificial Sequence
  EXG211-LmiR122-HmiR133
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggaattcccgggatatcgtcgacccacgcgtccgggccccacgctgcgcacccggggccaccatggccatgagcagcggaggaagcggaggaggagtgcccgagcaagaggacagcgtgctgtttaggagaggaaccggacagagcgatgactccgatatctgggacgacaccgctctgatcaaggcctatgacaaagccgtggcctccttcaagcacgctctgaagaatggcgatatctgtgagacctccggcaaacctaagaccacccccaagaggaagcccgccaagaagaacaagtcccagaagaagaataccgccgctagcctccagcagtggaaagtgggcgataagtgcagcgccatttggagcgaggatggatgcatctaccccgccaccattgccagcatcgacttcaagagggagacatgcgtggtggtgtataccggatacggaaatagagaggagcagaatctgagcgatctgctgtcccccatctgcgaggtggccaataatatcgagcagaacgcccaagagaacgagaacgaaagccaagtgtccaccgatgagagcgagaactccagaagccccggaaacaagtccgacaacatcaaacccaagagcgccccttggaacagctttctgcctcctcccccccccatgcccggccctagactgggacccggcaagcccggactgaagttcaacggacccccccctcctcctcccccccctcctcctcatctgctgagctgctggctcccccctttccctagcggcccccccattatccccccccctccccctatctgtcccgacagcctcgatgacgctgacgccctcggaagcatgctgatcagctggtacatgagcggctaccacaccggatactacatgggcttcagacagaaccagaaggagggcagatgctcccactctctgaactgaggagaaatgctggcatagagcagcactaaatgacaccactaaagaaacgatcagacagatctagtataacccgggccaaaagctcagtataacccgggccaaaagctcgacataattcgagcaaaaagctaacataattcgagcaaaaagctcttgacaaacaccattgtcacactccaacaaacaccattgtcacactccaacaaacaccattgtcacactccaccatagacagctggttgaaggggaccaaaacagctggttgaaggggaccaaaacagctggttgaaggggaccaaacaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 26 - protein, 735 aa, Artificial Sequence [sequence omitted]

SEQ ID NO: 27 - protein, 738 aa, Artificial Sequence
  AAV8 (Uniprot Q8JQF8_9VIRU)
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 28 - protein, 736 aa, Artificial Sequence
  AAVrh8 (Uniprot Q808Y3_9VIRU)
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 29 - protein, 736 aa, Artificial Sequence
  AAV9 (Uniprot Q6JC40_9VIRU)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 738 aa, Artificial Sequence
  AAVrh10 (Uniprot Q808W5_9VIRU)
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 31 - protein, 735 aa, Artificial Sequence
  AAV2v
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQGVSKVSADNNNSEFSWTGATKYHLNGRDSLVNPGPAMASHKDDEEKFFPQSGVLIFGKQGSEKTNVDIEKVMITDEEEIRTTNPVATEQYGSVSTNLQSGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 32 - protein, 736 aa, Artificial Sequence
  AAV44-9
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSNMASQARNWVPGPSYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 33 - protein, 294 aa, Artificial Sequence
  SMN protein (wt) amino acid sequence
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 34 - DNA, 882 bp, Artificial Sequence
  SMN nucleic acid sequence (wt)
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaat

SEQ ID NO: 35 - DNA, 882 bp, Artificial Sequence
  Codon optimized SMN nucleic acid sequence
atggccatgagcagcggaggaagcggaggaggagtgcccgagcaagaggacagcgtgctgtttaggagaggaaccggacagagcgatgactccgatatctgggacgacaccgctctgatcaaggcctatgacaaagccgtggcctccttcaagcacgctctgaagaatggcgatatctgtgagacctccggcaaacctaagaccacccccaagaggaagcccgccaagaagaacaagtcccagaagaagaataccgccgctagcctccagcagtggaaagtgggcgataagtgcagcgccatttggagcgaggatggatgcatctaccccgccaccattgccagcatcgacttcaagagggagacatgcgtggtggtgtataccggatacggaaatagagaggagcagaatctgagcgatctgctgtcccccatctgcgaggtggccaataatatcgagcagaacgcccaagagaacgagaacgaaagccaagtgtccaccgatgagagcgagaactccagaagccccggaaacaagtccgacaacatcaaacccaagagcgccccttggaacagctttctgcctcctcccccccccatgcccggccctagactgggacccggcaagcccggactgaagttcaacggacccccccctcctcctcccccccctcctcctcatctgctgagctgctggctcccccctttccctagcggcccccccattatccccccccctccccctatctgtcccgacagcctcgatgacgctgacgccctcggaagcatgctgatcagctggtacatgagcggctaccacaccggatactacatgggcttcagacagaaccagaaggagggcagatgctcccactctctgaac

SEQ ID NO: 36 - DNA, 850 bp, Artificial Sequence
  Exemplary Promoter Sequence (1)
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgct

SEQ ID NO: 37 - DNA, 796 bp, Artificial Sequence
  Exemplary Promoter Sequence (2)
ccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcagg

SEQ ID NO: 38 - DNA, 448 bp, Artificial Sequence
  Core promoter (hSyn) sequences
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 39 - DNA, 304 bp, Artificial Sequence
  CMV enhancer
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 40 - DNA, 420 bp, Artificial Sequence
  ProC3 enhancer
gatgaattccgccggaaactaggtccggaggactgccggaaacacctgtaatcaagccgccggaaacctgttgtggccgtatgccggaaacgtcttaattggacgtgccggaaactcttttaatgagttcgccggaaaccagaccagccgagctgccggaaaccggttatatagaacggccggaaacggtccacaggaaaaagccggaaacacccaaacggttagcgccggaaacgactggggaggacgtgccggaaacgtactatctgaagatgccggaaacacttgaaagctccaagccggaaacgggcccgtgcggatagccggaaactgacggtacacggccgccggaaacactacttgtatggtagccggaaacttgggcgtggctggggccggaaacgctcgagatctgcgatc

SEQ ID NO: 41 - DNA, 450 bp, Artificial Sequence
  ProA5 enhancer
cctggaggccttcctggaagaagagatcctggcaccgcacaaagagaagcacaggctttccagggctgaggagagggaggtcaagtgaggcccaggtgcccctgcctgagcctgtgtccccagaaacctcctctccctctcatcacccccacatcctccctgccactccccgcagctccctgtggccaagtgcactgcagcactcggctctgctccacaaacggtctgctccactccaggaaggccacctcctccccccccccccacctccggctgtcaccactcaccgctctagcctccagggggtggggaccccagagctggacacaccccatcgaagccccacagctcagccagccggacagactcacggtcggactcaagaccccggagccctgaggtgggcagcgcgccagggttcctcgcagcctcttcaaggtcagtgcaagt

SEQ ID NO: 42 - DNA, 472 bp, Artificial Sequence
  ProB15 enhancer
cagcccccgggccctcctcctccctctgcctttttaagggacgccctccagggcgaccccggagggcggacttgccaagctgaagagaatcagtcaaaaatccgcccacaggggacacatcatttaaataaatgtgtttctttgcccgaacagaagttcagataggctcgattatcattaattctgggtttcacgtaacgagaggaaacacaggttgcaataaaaataaaaaaatggtttgaaatcaattttaactcattttgaacgtcctcacacgtttgacaaaccgatttgtttcaggagacttgctaatatctaaatcggtgacagggtgtttgctgtgagtgtggctctggaaaagttattaagcgttataaaaaaaatgatgtaatgaaattctaattaatgggagggaagtgccaacaaatcactccttaaaatattaacgctatcaaagaacagctggagaagg

SEQ ID NO: 43 - DNA, 675 bp, Artificial Sequence
  ProC3 promoter
gaaacagctgagggtgcccagccggaaactcgaaatcaacgtaggccggaaactattcgatgaattccgccggaaactaggtccggaggactgccggaaacacctgtaatcaagccgccggaaacctgttgtggccgtatgccggaaacgtcttaattggacgtgccggaaactcttttaatgagttcgccggaaaccagaccagccgagctgccggaaaccggttatatagaacggccggaaacggtccacaggaaaaagccggaaacacccaaacggttagcgccggaaacgactggggaggacgtgccggaaacgtactatctgaagatgccggaaacacttgaaagctccaagccggaaacgggcccgtgcggatagccggaaactgacggtacacggccgccggaaacactacttgtatggtagccggaaacttgggcgtggctggggccggaaacgctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 44 - DNA, 1178 bp, Artificial Sequence
  EF1a promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 45 - DNA, 1984 bp, Artificial Sequence
  EXG301
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtgctgcgtatggcgtatagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcaagctgctagccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctacaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 46 - DNA, 2199 bp, Artificial Sequence
  EXG302
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaacgcgtaaagctggaactggggccggaaacagctgagggtgcccagccggaaactcgaaatcaacgtaggccggaaactattcgatgaattccgccggaaactaggtccggaggactgccggaaacacctgtaatcaagccgccggaaacctgttgtggccgtatgccggaaacgtcttaattggacgtgccggaaactcttttaatgagttcgccggaaaccagaccagccgagctgccggaaaccggttatatagaacggccggaaacggtccacaggaaaaagccggaaacacccaaacggttagcgccggaaacgactggggaggacgtgccggaaacgtactatctgaagatgccggaaacacttgaaagctccaagccggaaacgggcccgtgcggatagccggaaactgacggtacacggccgccggaaacactacttgtatggtagccggaaacttgggcgtggctggggccggaaacgctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggatccgccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctacaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 47 - DNA, 2685 bp, Artificial Sequence
  EXG303
cctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaattcacgcgtgtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctacaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 48 - DNA, 2452 bp, Artificial Sequence
  EXG304
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcaacgcgtaaagctggaactggggccggaaacagctgagggtgcccagccggaaactcgaaatcaacgtaggccggaaactattcgatgaattccgccggaaactaggtccggaggactgccggaaacacctgtaatcaagccgccggaaacctgttgtggccgtatgccggaaacgtcttaattggacgtgccggaaactcttttaatgagttcgccggaaaccagaccagccgagctgccggaaaccggttatatagaacggccggaaacggtccacaggaaaaagccggaaacacccaaacggttagcgccggaaacgactggggaggacgtgccggaaacgtactatctgaagatgccggaaacacttgaaagctccaagccggaaacgggcccgtgcggatagccggaaactgacggtacacggccgccggaaacactacttgtatggtagccggaaacttgggcgtggctggggccggaaacgctcgagatctgcgatcagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggatacacgccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctacaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 49 - DNA, 2451 bp, Artificial Sequence
  EXG305
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcgcgtgcccctgcctgcgcgagggcgggaagacagcccccgggccctcctcctccctctgcctttttaagggacgccctccagggcgaccccggagggcggacttgccaagctgaagagaatcagtcaaaaatccgcccacaggggacacatcatttaaataaatgtgtttctttgcccgaacagaagttcagataggctcgattatcattaattctgggtttcacgtaacgagaggaaacacaggttgcaataaaaataaaaaaatggtttgaaatcaattttaactcattttgaacgtcctcacacgtttgacaaaccgatttgtttcaggagacttgctaatatctaaatcggtgacagggtgtttgctgtgagtgtggctctggaaaagttattaagcgttataaaaaaaatgatgtaatgaaattctaattaatgggagggaagtgccaacaaatcactccttaaaatattaacgctatcaaagaacagctggagaaggagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggatacacgccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctacaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 50 - DNA, 2450 bp, Artificial Sequence
  EXG306
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcgcgtggtgcccaggcagtgggagcagggctgaccagagttctgcagagattgcctggaggccttcctggaagaagagatcctggcaccgcacaaagagaagcacaggctttccagggctgaggagagggaggtcaagtgaggcccaggtgcccctgcctgagcctgtgtccccagaaacctcctctccctctcatcacccccacatcctccctgccactccccgcagctccctgtggccaagtgcactgcagcactcggctctgctccacaaacggtctgctccactccaggaaggccacctcctccccccccccccacctccggctgtcaccactcaccgctctagcctccagggggtggggaccccagagctggacacaccccatcgaagccccacagctcagccagccggacagactcacggtcggactcaagaccccggagccctgaggtgggcagcgcgccagggttcctcgcagcctcttcaaggtcagtgcaagtagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggatacacgccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctacaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 51 - DNA, 2256 bp, Artificial Sequence
  EXG307
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcgcgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggatacacgccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctacaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 52 - DNA, 2256 bp, Artificial Sequence
  EXG340
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcgcgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggatacacgccaccatggccatgagcagcggaggaagcggaggaggagtgcccgagcaagaggacagcgtgctgtttaggagaggaaccggacagagcgatgactccgatatctgggacgacaccgctctgatcaaggcctatgacaaagccgtggcctccttcaagcacgctctgaagaatggcgatatctgtgagacctccggcaaacctaagaccacccccaagaggaagcccgccaagaagaacaagtcccagaagaagaataccgccgctagcctccagcagtggaaagtgggcgataagtgcagcgccatttggagcgaggatggatgcatctaccccgccaccattgccagcatcgacttcaagagggagacatgcgtggtggtgtataccggatacggaaatagagaggagcagaatctgagcgatctgctgtcccccatctgcgaggtggccaataatatcgagcagaacgcccaagagaacgagaacgaaagccaagtgtccaccgatgagagcgagaactccagaagccccggaaacaagtccgacaacatcaaacccaagagcgccccttggaacagctttctgcctcctcccccccccatgcccggccctagactgggacccggcaagcccggactgaagttcaacggacccccccctcctcctcccccccctcctcctcatctgctgagctgctggctcccccctttccctagcggcccccccattatccccccccctccccctatctgtcccgacagcctcgatgacgctgacgccctcggaagcatgctgatcagctggtacatgagcggctaccacaccggatactacatgggcttcagacagaaccagaaggagggcagatgctcccactctctgaactgaggagaaatgctggcatagagcagcactaaatgacaccactaaagaaacgatcagacagatctacaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 53 - DNA, 2503 bp, Artificial Sequence
  EXG341
ctgcgcgctcgctcgctcactgaggccgcccgggcaaagcccgggcgtcgggcgacctttggtcgcccggcctcagtgagcgagcgagcgcgcagagagggagtggaatgcacgcgtggatctgagttcgcgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggatacacgccaccatggccatgagcagcggaggaagcggaggaggagtgcccgagcaagaggacagcgtgctgtttaggagaggaaccggacagagcgatgactccgatatctgggacgacaccgctctgatcaaggcctatgacaaagccgtggcctccttcaagcacgctctgaagaatggcgatatctgtgagacctccggcaaacctaagaccacccccaagaggaagcccgccaagaagaacaagtcccagaagaagaataccgccgctagcctccagcagtggaaagtgggcgataagtgcagcgccatttggagcgaggatggatgcatctaccccgccaccattgccagcatcgacttcaagagggagacatgcgtggtggtgtataccggatacggaaatagagaggagcagaatctgagcgatctgctgtcccccatctgcgaggtggccaataatatcgagcagaacgcccaagagaacgagaacgaaagccaagtgtccaccgatgagagcgagaactccagaagccccggaaacaagtccgacaacatcaaacccaagagcgccccttggaacagctttctgcctcctcccccccccatgcccggccctagactgggacccggcaagcccggactgaagttcaacggacccccccctcctcctcccccccctcctcctcatctgctgagctgctggctcccccctttccctagcggcccccccattatccccccccctccccctatctgtcccgacagcctcgatgacgctgacgccctcggaagcatgctgatcagctggtacatgagcggctaccacaccggatactacatgggcttcagacagaaccagaaggagggcagatgctcccactctctgaactgaggagaaatgctggcatagagcagcactaaatgacaccactaaagaaacgatcagacagatctataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagtctagagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

(19) United States
(12) Patent Application Publication
(10) Pub. No.: US 2023/0293730 A1
(43) Pub. Date: Sep. 21, 2023

(54) NUCLEIC ACID CONSTRUCTS AND USES THEREOF FOR TREATING SPINAL MUSCULAR ATROPHY

(71) Applicants: HANGZHOU EXEGENESIS BIO LTD., Hangzhou, Zhejiang (CN); HANGZHOU JIAYIN BIOTECH LTD., Hangzhou, Zhejiang (CN)

(72) Inventors: Peixin ZHU, Lexington, MA (US); Guojie YE, Gainesville, FL (US); Zhenhua WU, Lansdale, PA (US); Lijun WANG, Brisbane, CA (US)

(73) Assignee: HANGZHOU EXEGENESIS BIO LTD., Hangzhou, Zhejiang (CN)

(21) Appl. No.: 18/019,449

(22) PCT Filed: Aug. 4, 2021
(86) PCT No.: PCT/CN2021/110521

§ 371 Date: Feb. 2, 2023

(30) Foreign Application Priority Data
Aug. 5, 2020 (WO) PCT/CN2020/107173
Dec. 21, 2020 (WO) PCT/CN2020/138056

(51) Int. Cl. A61K 48/00; C12N 15/86; A61K 38/17; A61P 21/00

(52) U.S. Cl. CPC A61K 48/0066; C12N 15/86; A61K 38/1709; A61K 48/0041; A61K 48/0058; A61P 21/00; C12N 2750/14143; C12N 2750/14145

(57) ABSTRACT

A nucleic acid comprising a first nucleic acid region comprising a nucleic acid sequence encoding a SMN protein or variant thereof; and a second nucleic acid region comprising one or more target segment(s) of one or more endogenous microRNA(s), wherein the second nucleic acid region is at 3′ of the first nucleic acid region.

### CROSS-REFERENCE TO RELATED APPLICATIONS

This application claims the benefit of PCT Application No. PCT/CN2020/107173, filed Aug. 5, 2020; and PCT Application No. PCT/CN2020/138056, filed Dec. 21, 2020, each of which is herein incorporated by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file “14652-017 228_SEQ_LISTING.txt” and a creation date of Jul. 21, 2021 and having a size of 108,587 bytes. The sequence listing submitted via EFS-Web is part of the specification and is herein incorporated by reference in its entirety.

### 1. FIELD

The present disclosure relates to nucleic acid constructs, gene therapies based on such constructs, and methods of use thereof.

### 2. BACKGROUND

Spinal muscular atrophy (SMA) is an autosomal recessive neurodegenerative disorder characterized by progressive muscular weakness and hypotonia as a consequence of the loss of lower motor neurons. On the basis of the age of onset and the severity of the neuromuscular symptoms, four clinical phenotypes have been described (Lunn and Wang, Lancet, 371 (9630):2120-33 (2008)). The most severe form, type 1 SMA, is a devastating childhood condition also known as Werdnig-Hoffmann disease. The gene responsible for most cases of SMA, survival motor neuron (SMN), was identified at the chromosomal locus 5q13 (Lefebvre et. al., Cell, 80(1):155-65 (1995)). The human gene is duplicated with telomeric and centromeric copies, SMN1 and SMN2, respectively. SMA is caused by mutations or deletion of the SMN1 gene, leading to depletion of SMN protein, because SMN2 fails to generate sufficient amounts of full-length protein.

Therapeutic strategies including gene threapies have been generated based on increasing expression of the SMN gene. However, current available treatments face various challenges including, for example, insufficient expression level of the SMN gene and off-targeting toxicitity. Thus, there is a need in the art for improved gene therapies for SMA with optimized SMN expression and reduced off-targeting toxicity.

### 3. SUMMARY

In one aspect, provided herein is a nucleic acid comprising (i) a first nucleic acid region comprising a nucleic acid sequence encoding a SMN protein or variant thereof; and (ii) a second nucleic acid region comprising one or more target segment(s) of one or more endogenous microRNA(s) (miRNA(s)), wherein the second nucleic acid region is at 3′ of the first nucleic acid region.

In some embodiments, the SMN protein or variant thereof comprises an amino acid sequence of SEQ ID NO: 33, or an amino acid sequence having at least 80%, 85%, 90%, 91%, 92%, 93%, 94%, 95%, 96%, 97%, 98%, 99% identity to SEQ ID NO: 33.

In some embodiments, the first nucleic acid region comprises a nucleic acid sequence selected from a group consisting of SEQ ID NO: 34 and SEQ ID NO: 35.

In some embodiments, the second nucleic acid region comprises at least one target segment of an endogenous miRNA in heart. In some embodiments, the endogenous miRNA in heart is selected from a group consisting of hsa-mir-1-5p, hsa-mir-208a-5p, hsa-mir-208b-5p, hsa-mir-133a-1, and hsa-mir-488-5p. In some embodiments, the endogenous miRNA in heart comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 1, SEQ ID NO: 2, SEQ ID NO: 3, SEQ ID NO: 5, or SEQ ID NO: 6.

In some embodiments, the second nucleic acid region comprises at least one target segment of an endogenous miRNA in liver. In some embodiments, the endogenous miRNA in liver is hsa-mir-122. In some embodiments, the endogenous miRNA in liver comprises a nucleic acid sequence at least 80%, 85%, 90%, 91%, 92%, 93%, 94%, 95%, 96%, 97%, 98%, 99% or 100% identical to SEQ ID NO: 4.

In some embodiments, the second nucleic acid region comprises two or more target segments of hsa-mir-133a-1. In some embodiments, the second nucleic acid region comprises at least three target segments of hsa-mir-133a-1. In some embodiments, the second nucleic acid region comprises at least one target segment of hsa-mir-208a-5p, at least one target segment of hsa-mir-208b-5p, at least one target segment of hsa-mir-122, and at least one target segment of hsa-mir-133a-1. In some embodiments, the second nucleic acid region comprises 2 target segments of hsa-mir-208a-5p, 2 target segments of hsa-mir-208b-5p, 3 target segments of hsa-mir-122, and 3 target segments of hsa-mir-133a-1.

In some embodiments, the target segment of hsa-mir-1-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 7; the target segment of hsa-mir-208a-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 8; the target segment of hsa-mir-208b-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 9; the target segment of hsa-mir-122 comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 10; the target segment of hsa-mir-133a-1 comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 11; and/or the target segment of hsa-mir-488-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 12.

In some embodiments, the second nucleic acid region comprises at least 3 repeats of a nucleic acid sequence of SEQ ID NO: 11. In some embodiments, the second nucleic acid region further comprises one or more target segments of an endogenous miRNA in liver.

In some embodiments, the second nucleic acid region comprises (i) 2 repeats of a target segment having a nucleic acid sequence of SEQ ID NO: 8, (ii) 2 repeats of a target segment having a nucleic acid sequence of SEQ ID NO: 9, (iii) 3 repeats of a target segment having a nucleic acid sequence of SEQ ID NO: 10, and (iv) 3 repeats of a target segment having a nucleic acid sequence of SEQ ID NO: 11.

In some embodiments, the second nucleic acid region further comprises one or more linkers between target segments. In some embodiments, the linker comprises 1 to 10 nucleotides. In some embodiments, the linker comprises a nucleic acid sequence selected from a group consisting of SEQ ID NO: 13, SEQ ID NO: 14, SEQ ID NO: 15, SEQ ID NO: 16, and SEQ ID NO: 17.

In some embodiments, the second nucleic acid region comprises: (i) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 18; (ii) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 19; (iii) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 20; or (iv) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 21.

In some embodiments, the first nucleic acid region further comprises a promoter. In some embodiments, the promoter comprises a nucleic acid sequence of SEQ ID NO: 36 or SEQ ID NO: 37. In some embodiments, the promoter comprises CMV enhancer and hSyn promoter. In some embodiments, the promoter comprises proC3 enhancer and hSyn promoter. In some embodiments, the promoter comprises proA5 enhancer and hSyn promoter. In other embodiments, the promoter comprises proB15 enhancer and hSyn promoter. In some embodiments, the promoter comprises a region of SEQ ID NO: 38 and a region of SEQ ID NO: 39. In some embodiments, the promoter comprises a region of SEQ ID NO: 38 and a region of SEQ ID NO: 40. In some embodiments, the promoter comprises a region of SEQ ID NO: 38 and a region of SEQ ID NO: 41. In some embodiments, the promoter comprises a region of SEQ ID NO: 38 and a region of SEQ ID NO: 42.

In another aspect, provided herein is a vector comprising the nucleic acid provided herein. In some embodiments, the vector is a viral vector. In some embodiments, the viral vector is an adeno-associated virus (AAV) vector. In some embodiments, the AAV vector is derived from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a combination or variant thereof. In a specific embodiment, the vector is a recombinant AAV9 (rAAV9) vector, or a variant thereof.

In yet another aspect, provided herein is a recombinant AAV (rAAV) vector comprising (i) a first nucleic acid region comprising a transgene; and (ii) a second nucleic acid region comprising one or more target segment(s) of one or more endogenous miRNA(s), wherein at least one target segment is a target segment of an endogenous miRNA in heart, and at least one target segment is a target segment of an endogenous miRNA in liver; wherein the second nucleic acid region is at 3′ of the first nucleic acid region; and wherein the rAAV vector comprises an inverted terminal repeat (ITR) from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, or AAV44-9.

In some embodiments, the first nucleic acid region comprises a nucleic acid sequence encoding a SMA protein or variant thereof comprising an amino acid sequence of SEQ ID NO: 33, or an amino acid sequence having at least 80%, 85%, 90%, 91%, 92%, 93%, 94%, 95%, 96%, 97%, 98%, 99% identity to SEQ ID NO: 33.

In some embodiments, the first nucleic acid region comprises a nucleic acid sequence selected from a group consisting of SEQ ID NO: 34 and SEQ ID NO: 35.

In some embodiments, the endogenous miRNA in heart is selected from a group consisting of hsa-mir-1-5p, hsa-mir-208a-5p, hsa-mir-208b-5p, hsa-mir-133a-1, and hsa-mir-488-5p; and/or wherein the endogenous miRNA in liver is hsa-mir-122.

In some embodiments, (i) the endogenous miRNA in heart comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 1, SEQ ID NO: 2, SEQ ID NO: 3, SEQ ID NO: 5, or SEQ ID NO: 6; and/or (ii) the endogenous miRNA in liver comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 4.

In some embodiments, the second nucleic acid region comprises two or more target segments of hsa-mir-133a-1. In some embodiments, the second nucleic acid region comprises at least three target segments of hsa-mir-133a-1. In some embodiments, the second nucleic acid region comprises at least one target segment of hsa-mir-208a-5p, at least one target segment of hsa-mir-208b-5p, at least one target segment of hsa-mir-122, and at least one target segment of hsa-mir-133a-1. In some embodiments, the second nucleic acid region comprises 2 target segments of hsa-mir-208a-5p, 2 target segments of hsa-mir-208b-5p, 3 target segments of hsa-mir-122, and 3 target segments of hsa-mir-133a-1.

In some embodiments, (i) the target segment of hsa-mir-1-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 7; (ii) the target segment of hsa-mir-208a-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 8; (iii) the target segment of hsa-mir-208b-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 9; (iv) the target segment of hsa-mir-122 comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 10; (v) the target segment of hsa-mir-133a-1 comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 11; and/or (vi) the target segment of hsa-mir-488-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 12.

In some embodiments, the second nucleic acid region comprises at least 3 repeats of a nucleic acid sequence of SEQ ID NO: 11.

In some embodiments, the second nucleic acid region comprises: (i) 2 repeats of a target segment having a nucleic acid sequence of SEQ ID NO: 8, (ii) 2 repeats of a target segment having a nucleic acid sequence of SEQ ID NO: 9, (iii) 3 repeats of a target segment having a nucleic acid sequence of SEQ ID NO: 10, and (iv) 3 repeats of a target segment having a nucleic acid sequence of SEQ ID NO: 11.

In some embodiments, the second nucleic acid region further comprises one or more linkers between target segments, and wherein optionally the linker comprises 1 to 1500 nucleotides, 1 to 500 nucleotides, 1 to 100 nucleotides, 1 to 50 nucleotides, or 1 to 10 nucleotides.

In some embodiments, the linker comprises a nucleic acid sequence selected from a group consisting of SEQ ID NO: 13, SEQ ID NO: 14, SEQ ID NO: 15, SEQ ID NO: 16, and SEQ ID NO: 17.

In some embodiments, the second nucleic acid region comprises: (i) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 18; (ii) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 19; (iii) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 20; or (ii) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 21.

In some embodiments, the first nucleic acid region further comprises a promoter. In some embodiments, the promoter comprises a nucleic acid sequence of SEQ ID NO: 36 or SEQ ID NO: 37. In some embodiments, the promoter comprises CMV enhancer and hSyn promoter. In some embodiments, the promoter comprises proC3 enhancer and hSyn promoter. In some embodiments, the promoter comprises proA5 enhancer and hSyn promoter. In other embodiments, the promoter comprises proB15 enhancer and hSyn promoter. In some embodiments, the promoter comprises a region of SEQ ID NO: 38 and a region of SEQ ID NO: 39. In some embodiments, the promoter comprises a region of SEQ ID NO: 38 and a region of SEQ ID NO: 40. In some embodiments, the promoter comprises a region of SEQ ID NO: 38 and a region of SEQ ID NO: 41. In some embodiments, the promoter comprises a region of SEQ ID NO: 38 and a region of SEQ ID NO: 42.

In some embodiments, the rAAV vector provided herein comprises an ITR is from AAV9.

In yet another aspect, provided herein is a nucleic acid comprising a nucleic acid region comprising a nucleic acid sequence encoding a SMN protein or variant thereof and a synthetic promoter comprising an enhancer and a core promoter, wherein optionally, (i) the synthetic promoter comprises CMV enhancer and hSyn promoter; (ii) the synthetic promoter comprises proC3 enhancer and hSyn promoter; (iii) the synthetic promoter comprises proA5 enhancer and hSyn promoter; or (iv) the synthetic promoter comprises proB15 enhancer and hSyn promoter.

In some embodiments, the hSyn promoter comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 38.

In some embodiments, the CMV enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 39.

In some embodiments, the proC3 enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 40.

In some embodiments, the proA5 enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 41.

In some embodiments, the proB15 enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 42.

In some embodiments, the SMN protein or variant thereof comprises an amino acid sequence of SEQ ID NO: 33, or an amino acid sequence having at least 80%, 85%, 90%, 91%, 92%, 93%, 94%, 95%, 96%, 97%, 98%, 99% identity to SEQ ID NO: 33.

In some embodiments, the nucleic acid sequence encoding the SMN protein or variant thereof comprises a nucleic acid sequence selected from a group consisting of SEQ ID NO: 34 and SEQ ID NO: 35.

In another aspect, provided herein is a rAAV vector comprising a nucleic acid sequence of SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53, or a nucleic acid sequence having at least 80%, 85%, 90%, 91%, 92%, 93%, 94%, 95%, 96%, 97%, 98%, 99% identify to SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

In another aspect, provided herein is a rAAV vector comprising a nucleic acid sequence of SEQ ID NO: 22 or SEQ ID NO: 23, SEQ ID NO: 24, or SEQ ID NO: 25, or a nucleic acid sequence having at least 80%, 85%, 90%, 91%, 92%, 93%, 94%, 95%, 96%, 97%, 98%, 99% identify to SEQ ID NO: 21, SEQ ID NO: 22, SEQ ID NO: 23, SEQ ID NO: 24 or SEQ ID NO: 25.

In yet another aspect, provided herein is a recombinant AAV (rAAV) particle comprising (a) the nucleic acid or the rAAV vector provided herein; and (b) a capsid protein of AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a variant thereof. In some embodiments, the capsid protein is a AAV9 capsid protein or a variant thereof.

In yet another aspect, provided herein is a pharmaceutical composition comprising the nucleic acid, the vector or rAAV vector, or the rAAV particle provided herein, and a pharmaceutically acceptable excipient.

In yet another aspect, provided herein is a method of enhancing expression of SMN protein in a cell, comprising contacting the cell with the nucleic acid, the vector or rAAV vector, the rAAV particle, or the pharmaceutical composition provided herein.

In yet another aspect, provided herein is a method of treating a disease or disorder in a subject, comprising administering to the subject the nucleic acid, the vector or rAAV vector, the rAAV particle, or the pharmaceutical composition provided herein. In some embodiments, the disease or disorder is a SMN associated disease or disorder. In some embodiments, the SMN associated disease or disorder is a disease or disorder associated with insufficient expression of SMN protein. In some embodiments, wherein the disease or disorder is associated with a deficient SMN protein. In other embodiments, the disease or disorder is associated with a smn1 gene deletion and/or mutation. In some embodiments, the disease or disorder is spinal muscular atrophy (SMA). In some embodiments, the disease or disorder is SMA-I, SMA-II, SMA-III, or SMA-IV. In some embodiments, the subject is under 2 years old.

### 4. BRIEF DESCRIPTION OF THE FIGURES

FIG. 1A illustrates the design of the exemplary rAAV vectors provided herein. FIG. 1B illustrates an exemplary nucleic acid region of the present nucleic acid constructs or rAAV vectors comprising multiple target segments.

FIG. 2A illustrates the SMN protein expression levels of various rAAV vectors provided herein. FIG. 2B shows the results of the transcriptome analysis for the codon optimizatized constructs provided herein.

FIG. 3 illustrates the survival results of the in vivo potency assay for the present rAAV particles.

FIG. 4 illustrates the open field activity results of the in vivo potency assay for the present rAAV particles.

FIG. 5 illustrates the body weight results of the in vivo potency assay for the present rAAV particles.

FIG. 6 illustrates the comparison of survival results among rAAV comprising different target segments of miRNAs.

FIGS. 7A and 7B illustrate the exemplary constructs provided herein comprising synthetic protomers.

FIG. 8A shows the in vivo potency assay results of the constructs comprising the synthetic promoter provided herein compared to constructs comprising other promoters.

FIG. 8B shows in vivo potency of constructs with various synthetic promoters with different enhancers at lower dosage.

### 5. DETAILED DESCRIPTION

The present disclosure is based in part on the novel nucleic acid constructs (e.g., nucleic acid constructs encoding a SMN protein and comprising a target sequence of a tissue specific microRNA), AAV vector comprising same, and improved properties thereof.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the polypeptides have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

As used herein, “nucleobase” is meant to refer to heterocyclic moiety capable of pairing with a base of another nucleic acid.

As used herein, “nucleotide” is meant to refer to a nucleoside having a phosphate group covalently linked to the sugar portion of the nucleoside.

As used herein, “nucleoside” is meant to refer to a nucleobase linked to a sugar.

The asymmetric ends of DNA and RNA strands are called the 5′ (five prime) and 3′ (three prime) ends, with the 5′ end having a terminal phosphate group and the 3′ end a terminal hydroxyl group. The five prime (5′) end has the fifth carbon in the sugar-ring of the deoxyribose or ribose at its terminus. Nucleic acids are synthesized in vivo in the 5′- to 3′-direction, because the polymerase used to assemble new strands attaches each new nucleotide to the 3′-hydroxyl (—OH) group via a phosphodiester bond.

An “isolated nucleic acid” is a nucleic acid, for example, an RNA, DNA, or a mixed nucleic acids, which is usually substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a polypeptide described herein is a nucleic acid molecule that is identified and separated from at least one contaminant nucleic acid molecule with which it is ordinarily associated in the environment in which it was produced.

The term “homology” as used herein refers to the percent identity between two polynucleotide or two polypeptide moieties. Two DNA, or two polypeptide sequences are “substantially homologous” to each other when the sequences exhibit at least about 50%, at least about 75%, at least about 80%-85%, at least about 90%, at least about 95%-98% sequence identity, at least about 99%, or any percent therebetween over a defined length of the molecules. As used herein, substantially homologous also refers to sequences showing complete identity to the specified DNA or polypeptide sequence.

The term “identity” as used herein refers to an exact nucleotide-to-nucleotide or amino acid-to-amino acid correspondence of two polynucleotides or polypeptide sequences, respectively. Methods for determining percent identity are well known in the art. For example, percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions. Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are well known in the art. Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The term “vector” as used herein refers to a substance that is used to carry or include a nucleic acid sequence, for example, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed both nucleic acid molecules can be inserted, for example, into a single expression vector or in separate expression vectors. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. The term “vector” includes cloning and expression vehicles, as well as viral vectors. In certain embodiments, the vector provided herein is a recombinant AAV vector.

The term “recombinant AAV vector (rAAV vector)” as used herein refers to a polynucleotide vector comprising a nucleic acid sequence from an AAV and one or more heterologous sequences (i.e., nucleic acid sequence not of AAV origin). In some embodiments, the one or more heterologous sequences are flanked by at least one, in certain embodiments two, AAV inverted terminal repeat sequences (ITRs). In some embodiments, such rAAV vectors can be replicated and packaged into infectious viral capsid particles, e.g., when present in a host cell that has been infected with a suitable helper virus (or that is expressing suitable helper functions) and that is expressing AAV rep and cap gene products (i.e., AAV Rep and Cap proteins). An rAAV vector may be incorporated into a larger polynucleotide (e.g., in a chromosome or in another vector such as a plasmid used for cloning or transfection), and can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral capsid particle, particularly an AAV particle. An rAAV vector can be packaged into an AAV capsid to generate a “recombinant adeno-associated viral capsid particle (rAAV particle).”

The term “heterologous” as used herein in connection with nucleic acid sequences such as coding sequences and control sequences, refers to sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene).

The term “flanked” as used herein with respect to a sequence that is flanked by other elements, indicates the presence of one or more the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence (e.g., a transgene) that is “flanked” by two other elements (e.g., TRs) indicates that one element is located 5′ to the sequence and the other is located 3′ to the sequence; however, there may be intervening sequences therebetween.

The term “inverted terminal repeat” or “ITR” sequence as used herein refers to relatively short sequences found at the termini of viral genomes which are in opposite orientation. An “AAV inverted terminal repeat (ITR)” sequence is well known in the art, and is usually an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “coding sequence” or a sequence which “encodes” a selected polypeptide, is a nucleic acid molecule which is transcribed (in the case of DNA) and translated (in the case of mRNA) into a polypeptide when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “promoter” as used herein in its ordinary sense refers to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters.”

The term “transgene” as used herein in a broad sense means any heterologous nucleotide sequence incorporated in a viral vector, e.g., for expression in a target cell and it can be associated with expression control sequences, such as promoters. It is appreciated by those of skill in the art that expression control sequences will be selected based on ability to promote expression of the transgene in the target cell. An example of a transgene is a nucleic acid encoding a therapeutic polypeptide or a detectable marker.

The term “AAV capsid” or “AAV capsid protein” or “AAV cap” as used herein refers to a protein encoded by an AAV capsid (cap) gene (e.g., VPI, VP2, and VP3) or a variant thereof. For example, the term includes but not limited to a capsid protein derived from any AAV serotype such as AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV-2/1, AAV 2/6, AAV 2/7, AAV 2/8, AAV 2/9, AAV LK03, AAVrh10, AAVrh74, AAV44-9, or a variant thereof. The term also includes a capsid protein expressed by or derived from a recombinant AAV such as a chimeric AAV.

The term “AAV capsid particle” or “AAV particle” as used herein includes at least one AAV capsid protein (e.g., a VP1 protein, a VP2 protein, a VP3 protein, or variant thereof) and optionally encapsulates a nucleic acid from an AAV genome or a nucleic acid derived from an AAV genome.

The term “serotype” used with respect to vector or virus capsid is defined by a distinct immunological profile based on the capsid protein sequences and capsid structure.

The term “chimeric” as used herein means, with respect to a viral capsid or particle, that the capsid or particle includes sequences from different parvoviruses, preferably different AAV serotypes, as described in Rabinowitz et al., U.S. Pat. No. 6,491,907 the disclosure of which is incorporated in its entirety herein by reference.

The term “recombinant” means a genetic entity distinct from that generally found in nature. As applied to a polynucleotide or gene, this means that the polynucleotide is the product of various combinations of cloning, restriction and/or ligation steps, and other procedures that result in the production of a construct that is distinct from a polynucleotide found in nature.

The term “recombinant virus” as used herein refers to a virus that has been genetically altered, e.g., by the addition or insertion of a heterologous nucleic acid construct into the particle. For example, the term “recombinant AAV particle” or “rAAV” as used herein refers to an AAV that has been genetically altered, e.g., by the deletion or other mutation of an endogenous AAV gene and/or the addition or insertion of a heterologous nucleic acid construct into the polynucleotide of the AAV particle.

As used herein, “detargeting activity” refers to any detectable or measurable activity attributable to the hybridization of an antisense compound to its target nucleic acid. In certain embodiments, detargeting activity is a decrease in the amount or expression of a target nucleic acid or its protein product encoded by such target nucleic acid.

As used herein, “endogenous miRNA” refers to a known or unknown microRNA, e.g., expressed by any mammalian cells, that is capable, e.g., after steps of processing, of undergoing hybridization to a target nucleic acid through hydrogen bonding, or protein/RNA complex. Non-limiting examples of endogenous miRNA include single-stranded and double-stranded DNA or RNA, or nuclei acid compounds, such as, antisense oligonucleotides, siRNAs, shRNAs, ssRNAs, miRNAs, lncRNAs and occupancy-based compounds.

As used herein, “detargeting inhibition” refers to reduction of target nucleic acid levels in the presence of an endogenous miRNA complementary to a target nucleic acid compared to target nucleic acid levels or in the absence of the endogenous miRNA.

As used herein, “antisense oligonucleotide” refers to a single-stranded oligonucleotide having a nucleobase sequence that permits hybridization to a corresponding segment of a target nucleic acid. According to some embodiments, the antisense oligonucleotides of the present disclosure comprise at least 80%, at least about 85%, at least about 90%, at least about 95% sequence complementarity to a target region within the target nucleic acid. For example, an endogenous miRNA in which 18 of 20 nucleobases of the antisense oligonucleotide are complementary, and would therefore specifically hybridize, to a target region would represent 90 percent complementarity. Percent complementarity of an endogenous miRNA with a region of a target nucleic acid can be determined routinely using basic local alignment search tools (BLAST programs) (see Altschul et al., J. Mol. Biol., 215, 403-410 (1990); Zhang and Madden, Genome Res., 7, 649-656 (1997)). In some embodiments, the antisense oligonucleotides include endogenous and other miRNAs, which hybridize to rAAV vector expressed SMN1 mRNA, and representative sequences of these endogenous miRNAs are described herein.

As used herein, “wildtype SMN1 transcript” means transcripts produced from the AAV vector containing the wildtype SMN1 mRNA with or without arrays of tissue specific target segments of miRNA. The wildtype SMN1 transcript with or without arrays of tissue specific target segments of miRNA therefore differs from the canonically transcribed mammalian cell “SMN1 sense transcript,” which is produced from the coding strand (also called sense strand) of the host cell smn1 gene.

As used herein, “specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

As used herein, “stringent hybridization conditions” or “stringent conditions” refers to conditions under which an oligomeric compound will hybridize to its target sequence, but to a minimal number of other sequences.

As used herein, a “target segment” refers to the sequence of nucleotides of a target nucleic acid to which an antisense compound (e.g., an miRNA) is targeted. “5′ target site” refers to the 5′-most nucleotide of a target segment. “3′ target site” is meant to refer to the 3′-most nucleotide of a target segment. A target segment of an miRNA is a nucleic acid sequence, whose mRNA transcript is specifically hybridizable by the miRNA.

The term “transfected” or “transformed” or “transduced” as used herein refers to a process by which exogenous nucleic acid is transferred or introduced into a host cell. A “transfected” or “transformed” or “transduced” cell is one which has been transfected, transformed or transduced with exogenous nucleic acid. For example, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous molecules into suitable host cells. “Transduction” of a cell by a virus means that there is transfer of a nucleic acid such as DNA or RNA from the virus particle to the cell.

The term “host cell” as used herein refers to a particular cell that may be transfected with a nucleic acid molecule and the progeny or potential progeny of such a cell. Host cells may be bacterial cells, yeast cells, insect cells or mammalian cell.

The term “purified” refers to isolation of a substance (compound, polynucleotide, protein, polypeptide, polypeptide composition) such that the substance of interest comprises the majority percent of the sample in which it resides. Typically in a sample a substantially purified component comprises 50%, 80%-85%, 90-99%, such as at least 90%, 91%, 92%, 93%, 94%, 95%, 96%, 97%, 98%, 99% of the sample. Techniques for purifying polynucleotides and polypeptides of interest are well-known in the art and include, for example, ion-exchange chromatography, affinity chromatography and sedimentation according to density.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease. “Treatment” or “treating” includes: (1) preventing the disease, i.e., preventing the development of the disease or causing the disease to occur with less intensity in a subject that may be exposed to or predisposed to the disease but does not yet experience or display symptoms of the disease, (2) inhibiting the disease, i.e., arresting the development, preventing or retarding progression, or reversing the disease state, (3) relieving symptoms of the disease i.e., decreasing the number of symptoms experienced by the subject, and (4) reducing, preventing or retarding progression of the disease or a symptom thereof. The terms “prevent,” “preventing,” and “prevention” refer to reducing the likelihood of the onset (or recurrence) of a disease, disorder, condition, or associated symptom(s).

As used herein, “administer”, “administration”, or “administering” refers to the act of injecting or otherwise physically delivering a substance (e.g., a conjugate or pharmaceutical composition provided herein) to a subject or a patient (e.g., human), such as by oral, mucosal, topical, intradermal, parenteral, intravenous, intravitreal, intraarticular, subretinal, intramuscular, intrathecal delivery and/or any other method of physical delivery described herein or known in the art. In a particular embodiment, administration is by intravenous infusion. A conjugate or a composition provided herein may be delivered systemically or to a specific tissue.

As used herein, the terms “effective amount” or “therapeutically effective amount” refer to an amount of a therapeutic (e.g., a conjugate or pharmaceutical composition provided herein) which is sufficient to treat, diagnose, prevent, delay the onset of, reduce and/or ameliorate the severity and/or duration of a given condition, disorder or disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy or to serve as a bridge to another therapy. In some embodiments, “effective amount” as used herein also refers to the amount of a conjugate described herein to achieve a specified result. As used herein, the terms “subject” and “patient” are used interchangeably.

As used herein, a subject is a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

As used herein, the terms “therapies” and “therapy” can refer to any protocol(s), method(s), compositions, formulations, and/or agent(s) that can be used in the prevention, treatment, management, or amelioration of a disease or disorder or symptom thereof (e.g., a disease or disorder provided herein or one or more symptoms or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a disease or disorder or one or more symptoms thereof. In certain embodiments, the term “therapy” refers to a therapy other than a conjugate described herein or pharmaceutical composition thereof.

As used herein, the term “disease or disorder associated with SMN” refers to a disease or disorder that involves SMN (including abnormal expression level of a SMN protein), e.g., either as a symptom or direct or indirect cause. A disease or disorder associated with SMN includes, but not limited to, a disease or disorder associated with decreased expression of smn1 gene or with a mutant smn1 gene.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Nucleic Acid Constructs

In one aspect, provided herein are novel nucleic acids comprising a first nucleic acid region encoding a protein of interest and a second nucleic acid region comprising one or more target segment(s) of one or more endogenous miRNA(s) (also referred to as a target sequence of miRNA).

In some embodiments, the first nucleic acid region encodes a therapeutic molecule (e.g., a SMN protein). In some embodiments, the second nucleic acid comprising one or more target segments is at 3′ of the first nucleic acid region encoding a protein of interest. In some embodiments, the second nucleic acid region comprising one or more target segments are immediately following or continuous to the 3′ of the first nucleic acid region encoding a protein of interest. In other embodiments, the second nucleic acid region comprising one or more target segments is spaced from the 3′ of the first nucleic acid region encoding a protein of interest with one or more nucleotides (e.g., 1, 2, 3, 4, 5, 6, 7, 8, 9, 10 or more nucleotides).

5.2.1. Nucleic Acids Encoding a Protein of Interest or Variants Thereof

In some embodiments, the nucleic acid constructs provided herein comprises a nucleic acid sequence encoding a SMN protein or a variant thereof.

In some embodiments, the nucleic acid constructs provided herein comprises a nucleic acid sequence encoding a SMN protein having an amino acid sequence of SEQ ID NO: 33. In some embodiments, the nucleic acid sequence encoding the SMN protein is selected from a group consisting of SEQ ID NO: 34 and SEQ ID NO: 35.

In some embodiments, the nucleic acids provided herein comprise certain percent identity to the nucleic acid sequence of SEQ ID NO: 34 or SEQ ID NO: 35. In some embodiments, the nucleic acid encoding a protein of interest provided herein comprises at least about any one of 80%, 85%, 86%, 87%, 88%, 89%, 90%, 91%, 92%, 93%, 94%, 95%, 96%, 97%, 98%, 99%, or 100% sequence identity to SEQ ID NO: 34. In some embodiments, the nucleic acid encoding a protein of interest provided herein comprises at least about any one of 80%, 85%, 86%, 87%, 88%, 89%, 90%, 91%, 92%, 93%, 94%, 95%, 96%, 97%, 98%, 99%, or 100% sequence identity to SEQ ID NO: 35.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

According to some embodiments, and of the sequences described herein can further comprise one or more modifications to a sugar moiety, an internucleoside linkage, or a nucleobase.

According to certain embodiments, the nucleic acid is a human nucleic acid (i.e., a nucleic acid that is derived from a human smn1 gene). In other embodiments, the nucleic acid is a non-human nucleic acid (i.e., a nucleic acid that is derived from a non-human smn1 gene).

According to some embodiments, the nucleic acid provided herein comprises one or more insertions, deletions, inversions, and/or substitutions. According to some embodiments, the nucleic acid construct provided herein comprises a nucleic acid region which has been codon optimized. According to one embodiment, the nucleic acid encoding smn1 is codon optimized. According to one embodiment, the nucleic acid encoding smn1 is codon optimized for expression in a eukaryote, e.g., humans. According to some embodiments, a coding sequence encoding smn1 is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al., Nucl. Acids Res. 28:292 (2000). Computer algorithms for codon optimizing a particular sequence for expression in a particular host cell are also available, such as Gene Forge (Aptagen; Jacobus, Pa.), are also available.

A nucleic acid molecule (including, for example, a smn1 nucleic acid) of the present disclosure can be isolated using standard molecular biology techniques. Using all or a portion of a nucleic acid sequence of interest as a hybridization probe, nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

A nucleic acid molecule for use in the methods of the disclosure can also be isolated by the polymerase chain reaction (PCR) using synthetic oligonucleotide primers designed based upon the sequence of a nucleic acid molecule of interest. A nucleic acid molecule used in the methods of the disclosure can be amplified using cDNA, mRNA or, alternatively, genomic DNA as a template and appropriate oligonucleotide primers according to standard PCR amplification techniques.

Furthermore, oligonucleotides corresponding to nucleotide sequences of interest can also be chemically synthesized using standard techniques. Numerous methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein). Automated methods for designing synthetic oligonucleotides are available. See e.g., Hoover, D. M. & Lubowski, J. Nucleic Acids Research, 30(10): e43 (2002).

In other embodiments, the nucleic acid constructs provided herein comprises a nucleic acid sequence encoding a SMN protein variant. In some embodiments, the SMN protein variant comprises an amino acid sequence having at least 80%, 85%, 90%, 91%, 92%, 93%, 94%, 95%, 96%, 97%, 98%, 99% identify to SEQ ID NO: 33.

It is contemplated that variants of the protein described herein can be prepared. For example, peptide variants can be prepared by introducing appropriate nucleotide changes into the encoding DNA, and/or by synthesis of the desired polypeptide. Those skilled in the art who appreciate that amino acid changes may alter post-translational processes of the peptide.

Variations may be a substitution, deletion, or insertion of one or more codons encoding the polypeptide that results in a change in the amino acid sequence as compared with the original polypeptide.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental peptides.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include a polypeptide with an N-terminal methionyl residue.

Proteins generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties. Exemplary substitutions are shown in the table below.

TABLE 1
Amino Acid Substitutions

| Original | Exemplary |
|---|---|
| Residue | Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the polypeptide provided herein also may be substituted, for example, with another amino acid, such as alanine or serine, to improve the oxidative stability of the molecule and to prevent aberrant crosslinking.

Non-conservative substitutions will entail exchanging a member of one of these classes for another class.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include a polypeptide with an N-terminal methionyl residue.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the polypeptide variant DNA.

In another aspect, provided herein is an optimized nucleic acid encoding a SMN protein comprising a nucleic acid sequence of SEQ ID NO: 35, or a nucleic acid sequence having at least 90%, 91%, 92%, 93%, 94%, 95%, 96%, 97%, 98% or 99% identity to SEQ ID NO: 35. In some embodiments, the optimized nucleic acid provided herein further comprises a promoter region comprising SEQ ID NO: 36 or SEQ ID NO: 37. In some specific embodiments, provided herein is a nucleic acid comprising a nucleic acid of SEQ ID NO: 36 and a nucleic acid of SEQ ID NO: 35. In other specific embodiments, provided herein is a nucleic acid comprising a nucleic acid of SEQ ID NO: 37 and a nucleic acid of SEQ ID NO: 35.

5.2.2. Target Segments of Endogenous miRNAs

In certain embodiments, the nucleic acid constructs provided herein comprise, in addition to the nucleic acid encoding a protein of interest (e.g., a SMN protein), a nucleic acid (or a target sequence of miRNA) comprising one or more target segment(s) of one or more endogenous miRNA(s). As shown in the Section 6 below, inclusion of certain target segment(s) of one or more endogenous miRNA(s) showed advantageous properties for reducing off-target toxicity for gene therapies (e.g., AAV based gene therapies).

In some embodiments, the nucleic acid comprises target segments of two or more endogenous miRNAs, and the two or more endogenous miRNA(s) are tissue specific miRNA(s). As used herein, a tissue-specific miRNA is an miRNA whose amount is higher is one or more specific tissues as compared to others, such as liver specific miRNAs and heart specific miRNAs. In some embodiments, the two or more endogenous miRNAs are specific to the same tissue. In other embodiments, the two or more endogenous miRNAs are specific to different tissues. In some specific embodiments, the nucleic acid comprises one or more target segment(s) of one or more liver specific miRNA(s) and one or more target segment(s) of one or more heart specific miRNA(s).

In some embodiments, the nucleic acid comprises 1 to 50 target segments. In some embodiments, the nucleic acid comprises 1 to 40 target segments. In some embodiments, the nucleic acid comprises 1 to 30 target segments. In some embodiments, the nucleic acid comprises 1 to 20 target segments. In some embodiments, the nucleic acid comprises 1 to 15 target segments. In some embodiments, the nucleic acid comprises 5 to 15 target segments. In some embodiments, the nucleic acid comprises 7 to 11 target segments.

A target segment of an miRNA refers to a nucleic acid segment specifically hybridizable or complementary to the miRNA (e.g., a tissue specific endogenous miRNA). The most common mechanism of hybridization involves hydrogen bonding (e.g., Watson-Crick, Hoogsteen or reversed Hoogsteen hydrogen bonding) between complementary nucleobases of the nucleic acid molecules. Stringent conditions are sequence-dependent and are determined by the nature and composition of the nucleic acid molecules to be hybridized. Methods of determining whether a sequence is specifically hybridizable to another sequence are well known in the art.

An endogenous miRNA and a target nucleic acid are complementary to each other when a sufficient number of nucleobases of the endogenous miRNA can hydrogen bond with the corresponding nucleobases of the target nucleic acid, such that a desired effect will occur (e.g., antisense inhibition of a target nucleic acid, such as a smn1 with target segments provided herein).

Non-complementary nucleobases between an miRNA and a smn1 with target segments may be tolerated provided that the miRNA remains able to specifically hybridize to a target nucleic acid. Further, an miRNA may hybridize over one or more segments of a smn1 with target segments such that intervening or adjacent segments are not involved in the hybridization event (e.g., a loop structure, mismatch or hairpin structure).

According to some embodiments, the miRNA provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to the target sequence of miRNA provided in the present nucleic acid constructs or a portion thereof. Percent complementarity of an miRNA with a target nucleic acid can be determined using routine methods. For example, an miRNA in which 18 of 20 nucleobases of the miRNA are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, an miRNA which is 18 nucleobases in length having 4 (four) non-complementary nucleobases which are flanked by two regions of complete complementarity with the target nucleic acid would have 77.8% overall complementarity with the target nucleic acid and would thus fall within the scope of the present disclosure. Percent complementarity of an miRNA with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In some embodiments, the miRNAs provided herein, or specified portions thereof, are fully complementary (i.e., 100% complementary) to a target nucleic acid, or specified portion thereof. For example, in some embodiments, an miRNA may be fully complementary to a target segment provided herein. As used herein, “fully complementary” means each nucleobase of an miRNA is capable of precise base pairing with the corresponding nucleobases of a target nucleic acid. For example, a 20-nucleobase miRNA is fully complementary to a target sequence that is 400 nucleobases long, so long as there is a corresponding 20 nucleobase portion of the target nucleic acid that is fully complementary to the miRNA. Fully complementary can also be used in reference to a specified portion of the first and/or the second nucleic acid. For example, a 20-nucleobase portion of a 30 nucleobase miRNA can be “fully complementary” to a target sequence that is 400 nucleobases long. The 20-nucleobase portion of the 30 nucleobase oligonucleotide is fully complementary to the target sequence if the target sequence has a corresponding 20 nucleobase portion wherein each nucleobase is complementary to the 20 nucleobase portion of the miRNA. At the same time, the entire 30 nucleobase miRNA may or may not be fully complementary to the target sequence, depending on whether the remaining 10 nucleobases of the miRNA are also complementary to the target sequence.

The location of a non-complementary nucleobase may be at the 5′ end or 3′ end, or anywhere in between of the 5′ end and 3′ end of the miRNA. Alternatively, the non-complementary nucleobase or nucleobases may be at an internal position of the miRNA. When two or more non-complementary nucleobases are present, they may be contiguous (i.e., linked) or non-contiguous. In one embodiment, a non-complementary nucleobase is located in the wing segment of a gapmer antisense oligonucleotide.

According to some embodiments, miRNAs that are, or are up to 12, 13, 14, 15, 16, 17, 18, 19, 20, 21, 22, 23, 24, 25, 26, 27, 28, 29, or 30 nucleobases in length comprise no more than 8, no more than 7, no more than 6, no more than 5, no more than 4, no more than 3, no more than 2, or no more than 1 non-complementary nucleobase(s) relative to a target nucleic acid. According to some embodiments, miRNAs that are, or are up to 12, 13, 14, 15, 16, 17, 18, 19, 20, 21, 22, 23, 24, 25, 26, 27, 28, 29, or 30 nucleobases in length comprise no more than 8, no more than 7, no more than 6, no more than 5, no more than 4, no more than 3, no more than 2, or no more than 1 non-complementary nucleobase(s) relative to a target nucleic acid.

The miRNAs provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an miRNA. According to some embodiments, the miRNAs, are complementary to at least an 8 nucleobase portion of a target segment. According to some embodiments, the miRNAs are complementary to at least a 9 nucleobase portion of a target segment. According to some embodiments, the miRNAs are complementary to at least a 10 nucleobase portion of a target segment. According to some embodiments, the miRNAs, are complementary to at least an 11 nucleobase portion of a target segment. According to some embodiments, the miRNAs, are complementary to at least a 12 nucleobase portion of a target segment. According to some embodiments, the miRNAs, are complementary to at least a 13 nucleobase portion of a target segment. According to some embodiments, the miRNAs, are complementary to at least a 14 nucleobase portion of a target segment. According to some embodiments, the miRNAs, are complementary to at least a 15 nucleobase portion of a target segment. Also contemplated are miRNAs that are complementary to at least a 9, 10, 11, 12, 13, 14, 15, 16, 17, 18, 19, 20, or more nucleobase portion of a target segment, or a range defined by any two of these values.

Non-limiting exemplary endogenous miRNAs and non-limiting exemplary target segments of these exemplary miRNAs are shown in Table 2 below.

TABLE 2
List of miRNA and Targeting Sequences Thereof

| miRNA | | |
|---|---|---|
| (hsa-) | miRNA sequence | Target Segments |
| mir-1-5p | 5′-UGGGAAACAUACUUC | 5′-atgggca |
| (or | UUUAUAUGCCCAUAU | tataaag |
| mir-1) | GGACCUGCUAAGCUA | aagtatg |
| | UGGAAUGUAAAGAAG | t-3′ |
| | UAUGUAUCUCA-3′ | (SEQ ID NO: 7) |
| | (SEQ ID NO: 1) | |
| mir-208a-5p | 5′-UGACGGGCGAGCUUU | 5′-gtataac |
| (or | UGGCCCGGGUUAUAC | ccgggcc |
| mir-208a) | CUGAUGCUCACGUAU | aaaagct |
| | AAGACGAGCAAAAAG | c-3′ |
| | CUUGUUGGUCA-3′ | (SEQ ID NO: 8) |
| | (SEQ ID NO: 2) | |
| mir-208b-5p | 5′-CCUCUCAGGGAAGCU | 5′-acataat |
| (or | UUUUGCUCGAAUUAU | tcgagca |
| mir-208b) | GUUUCUGAUCCGAAU | aaaagct |
| | AUAAGACGAACAAAA | -3′ |
| | GGUUUGUCUGAGGGC | (SEQ ID NO: 9) |
| | AG-3′ | |
| | (SEQ ID NO: 3) | |
| mir-122 | 5′-CCUUAGCAGAGCUGU | 5′-caaacac |
| | GGAGUGUGACAAUGG | cattgtc |
| | UGUUUGUGUCUAAAC | acactcc |
| | UAUCAAACGCCAUUA | a-3′ |
| | UCACACUAAAUAGCU | (SEQ ID NO: 10) |
| | ACUGCUAGGC-3′ | |
| | (SEQ ID NO: 4) | |
| mir-133a-1 | 5′-ACAAUGCUUUGCUAG | 5′-cagctgg |
| (or mir-133a) | AGCUGGUAAAAUGGA | ttgaagg |
| | ACCAAAUCGCCUCUU | ggaccaa |
| | CAAUGGAUUUGGUCC | a-3′ |
| | CCUUCAACCAGCUGU | (SEQ ID NO: 11) |
| | AGCUAUGCAUUGA-3′ | |
| | (SEQ ID NO: 5) | |
| mir-488-5p | 5′-GAGAAUCAUCUCUCC | 5′-ttgagag |
| (or | CAGAUAAUGGCACUC | tgccatt |
| mir-488) | UCAAACAAGUUUCCA | atctggg |
| | AAUUGUUUGAAAGGC | -3′ |
| | UAUUUCUUGGUCAGA | (SEQ ID NO: 12) |
| | UGACUCUC-3′ | |
| | (SEQ ID NO: 6) | |

In some embodiments, the endogenous miRNA sequence comprises SEQ ID NO: 1. In some embodiments, the endogenous miRNA sequence is at least 80% identical to SEQ ID NO: 1. In some embodiments, the endogenous miRNA sequence is about 80% identical to SEQ ID NO: 1. In some embodiments, the endogenous miRNA sequence is about 81% identical to SEQ ID NO: 1. In some embodiments, the endogenous miRNA sequence is about 82% identical to SEQ ID NO: 1. In some embodiments, the endogenous miRNA sequence is about 83% identical to SEQ ID NO: 1. In some embodiments, the endogenous miRNA sequence is about 84% identical to SEQ ID NO: 1. In some embodiments, the endogenous miRNA sequence is about 85% identical to SEQ ID NO: 1. In some embodiments, the endogenous miRNA sequence is about 86% identical to SEQ ID NO: 1. In some embodiments, the endogenous miRNA sequence is about 87% identical to SEQ ID NO: 1. In some embodiments, the endogenous miRNA sequence is about 88% identical to SEQ ID NO: 1. In some embodiments, the endogenous miRNA sequence is about 89% identical to SEQ ID NO: 1. In some embodiments, the endogenous miRNA sequence is about 90% identical to SEQ ID NO: 1. In some embodiments, the endogenous miRNA sequence is about 91% identical to SEQ ID NO: 1. In some embodiments, the endogenous miRNA sequence is about 92% identical to SEQ ID NO: 1. In some embodiments, the endogenous miRNA sequence is about 93% identical to SEQ ID NO: 1. In some embodiments, the endogenous miRNA sequence is about 94% identical to SEQ ID NO: 1. In some embodiments, the endogenous miRNA sequence is about 95% identical to SEQ ID NO: 1. In some embodiments, the endogenous miRNA sequence is about 96% identical to SEQ ID NO: 1. In some embodiments, the endogenous miRNA sequence is about 97% identical to SEQ ID NO: 1. In some embodiments, the endogenous miRNA sequence is about 98% identical to SEQ ID NO: 1. In some embodiments, the endogenous miRNA sequence is about 99% identical to SEQ ID NO: 1. In some embodiments, the nucleic acid comprises one or more target segment(s) of any of the above miRNAs.

In some embodiments, the endogenous miRNA sequence comprises SEQ ID NO: 2. In some embodiments, the endogenous miRNA sequence is at least 80% identical to SEQ ID NO: 2. In some embodiments, the endogenous miRNA sequence is about 80% identical to SEQ ID NO: 2. In some embodiments, the endogenous miRNA sequence is about 81% identical to SEQ ID NO: 2. In some embodiments, the endogenous miRNA sequence is about 82% identical to SEQ ID NO: 2. In some embodiments, the endogenous miRNA sequence is about 83% identical to SEQ ID NO: 2. In some embodiments, the endogenous miRNA sequence is about 84% identical to SEQ ID NO: 2. In some embodiments, the endogenous miRNA sequence is about 85% identical to SEQ ID NO: 2. In some embodiments, the endogenous miRNA sequence is about 86% identical to SEQ ID NO: 2. In some embodiments, the endogenous miRNA sequence is about 87% identical to SEQ ID NO: 2. In some embodiments, the endogenous miRNA sequence is about 88% identical to SEQ ID NO: 2. In some embodiments, the endogenous miRNA sequence is about 89% identical to SEQ ID NO: 2. In some embodiments, the endogenous miRNA sequence is about 90% identical to SEQ ID NO: 2. In some embodiments, the endogenous miRNA sequence is about 91% identical to SEQ ID NO: 2. In some embodiments, the endogenous miRNA sequence is about 92% identical to SEQ ID NO: 2. In some embodiments, the endogenous miRNA sequence is about 93% identical to SEQ ID NO: 2. In some embodiments, the endogenous miRNA sequence is about 94% identical to SEQ ID NO: 2. In some embodiments, the endogenous miRNA sequence is about 95% identical to SEQ ID NO: 2. In some embodiments, the endogenous miRNA sequence is about 96% identical to SEQ ID NO: 2. In some embodiments, the endogenous miRNA sequence is about 97% identical to SEQ ID NO: 2. In some embodiments, the endogenous miRNA sequence is about 98% identical to SEQ ID NO: 2. In some embodiments, the endogenous miRNA sequence is about 99% identical to SEQ ID NO: 2. In some embodiments, the nucleic acid comprises one or more target segment(s) of any of the above miRNAs.

In some embodiments, the endogenous miRNA sequence comprises SEQ ID NO: 3. In some embodiments, the endogenous miRNA sequence is at least 80% identical to SEQ ID NO: 3. In some embodiments, the endogenous miRNA sequence is about 80% identical to SEQ ID NO: 3. In some embodiments, the endogenous miRNA sequence is about 81% identical to SEQ ID NO: 3. In some embodiments, the endogenous miRNA sequence is about 82% identical to SEQ ID NO: 3. In some embodiments, the endogenous miRNA sequence is about 83% identical to SEQ ID NO: 3. In some embodiments, the endogenous miRNA sequence is about 84% identical to SEQ ID NO: 3. In some embodiments, the endogenous miRNA sequence is about 85% identical to SEQ ID NO: 3. In some embodiments, the endogenous miRNA sequence is about 86% identical to SEQ ID NO: 3. In some embodiments, the endogenous miRNA sequence is about 87% identical to SEQ ID NO: 3. In some embodiments, the endogenous miRNA sequence is about 88% identical to SEQ ID NO: 3. In some embodiments, the endogenous miRNA sequence is about 89% identical to SEQ ID NO: 3. In some embodiments, the endogenous miRNA sequence is about 90% identical to SEQ ID NO: 3. In some embodiments, the endogenous miRNA sequence is about 91% identical to SEQ ID NO: 3. In some embodiments, the endogenous miRNA sequence is about 92% identical to SEQ ID NO: 3. In some embodiments, the endogenous miRNA sequence is about 93% identical to SEQ ID NO: 3. In some embodiments, the endogenous miRNA sequence is about 94% identical to SEQ ID NO: 3. In some embodiments, the endogenous miRNA sequence is about 95% identical to SEQ ID NO: 3. In some embodiments, the endogenous miRNA sequence is about 96% identical to SEQ ID NO: 3. In some embodiments, the endogenous miRNA sequence is about 97% identical to SEQ ID NO: 3. In some embodiments, the endogenous miRNA sequence is about 98% identical to SEQ ID NO: 3. In some embodiments, the endogenous miRNA sequence is about 99% identical to SEQ ID NO: 3. In some embodiments, the nucleic acid comprises one or more target segment(s) of any of the above miRNAs.

In some embodiments, the endogenous miRNA sequence comprises SEQ ID NO: 4. In some embodiments, the endogenous miRNA sequence is at least 80% identical to SEQ ID NO: 4. In some embodiments, the endogenous miRNA sequence is about 80% identical to SEQ ID NO: 4. In some embodiments, the endogenous miRNA sequence is about 81% identical to SEQ ID NO: 4. In some embodiments, the endogenous miRNA sequence is about 82% identical to SEQ ID NO: 4. In some embodiments, the endogenous miRNA sequence is about 83% identical to SEQ ID NO: 4. In some embodiments, the endogenous miRNA sequence is about 84% identical to SEQ ID NO: 4. In some embodiments, the endogenous miRNA sequence is about 85% identical to SEQ ID NO: 4. In some embodiments, the endogenous miRNA sequence is about 86% identical to SEQ ID NO: 4. In some embodiments, the endogenous miRNA sequence is about 87% identical to SEQ ID NO: 4. In some embodiments, the endogenous miRNA sequence is about 88% identical to SEQ ID NO: 4. In some embodiments, the endogenous miRNA sequence is about 89% identical to SEQ ID NO: 4. In some embodiments, the endogenous miRNA sequence is about 90% identical to SEQ ID NO: 4. In some embodiments, the endogenous miRNA sequence is about 91% identical to SEQ ID NO: 4. In some embodiments, the endogenous miRNA sequence is about 92% identical to SEQ ID NO: 4. In some embodiments, the endogenous miRNA sequence is about 93% identical to SEQ ID NO: 4. In some embodiments, the endogenous miRNA sequence is about 94% identical to SEQ ID NO: 4. In some embodiments, the endogenous miRNA sequence is about 95% identical to SEQ ID NO: 4. In some embodiments, the endogenous miRNA sequence is about 96% identical to SEQ ID NO: 4. In some embodiments, the endogenous miRNA sequence is about 97% identical to SEQ ID NO: 4. In some embodiments, the endogenous miRNA sequence is about 98% identical to SEQ ID NO: 4. In some embodiments, the endogenous miRNA sequence is about 99% identical to SEQ ID NO: 4. In some embodiments, the nucleic acid comprises one or more target segment(s) of any of the above miRNAs.

In some embodiments, the endogenous miRNA sequence comprises SEQ ID NO: 5. In some embodiments, the endogenous miRNA sequence is at least 80% identical to SEQ ID NO: 5. In some embodiments, the endogenous miRNA sequence is about 80% identical to SEQ ID NO: 5. In some embodiments, the endogenous miRNA sequence is about 81% identical to SEQ ID NO: 5. In some embodiments, the endogenous miRNA sequence is about 82% identical to SEQ ID NO: 5. In some embodiments, the endogenous miRNA sequence is about 83% identical to SEQ ID NO: 5. In some embodiments, the endogenous miRNA sequence is about 84% identical to SEQ ID NO: 5. In some embodiments, the endogenous miRNA sequence is about 85% identical to SEQ ID NO: 5. In some embodiments, the endogenous miRNA sequence is about 86% identical to SEQ ID NO: 5. In some embodiments, the endogenous miRNA sequence is about 87% identical to SEQ ID NO: 5. In some embodiments, the endogenous miRNA sequence is about 88% identical to SEQ ID NO: 5. In some embodiments, the endogenous miRNA sequence is about 89% identical to SEQ ID NO: 5. In some embodiments, the endogenous miRNA sequence is about 90% identical to SEQ ID NO: 5. In some embodiments, the endogenous miRNA sequence is about 91% identical to SEQ ID NO: 5. In some embodiments, the endogenous miRNA sequence is about 92% identical to SEQ ID NO: 5. In some embodiments, the endogenous miRNA sequence is about 93% identical to SEQ ID NO: 5. In some embodiments, the endogenous miRNA sequence is about 94% identical to SEQ ID NO: 5. In some embodiments, the endogenous miRNA sequence is about 95% identical to SEQ ID NO: 5. In some embodiments, the endogenous miRNA sequence is about 96% identical to SEQ ID NO: 5. In some embodiments, the endogenous miRNA sequence is about 97% identical to SEQ ID NO: 5. In some embodiments, the endogenous miRNA sequence is about 98% identical to SEQ ID NO: 5. In some embodiments, the endogenous miRNA sequence is about 99% identical to SEQ ID NO: 5. In some embodiments, the nucleic acid comprises one or more target segment(s) of any of the above miRNAs.

In some embodiments, the endogenous miRNA sequence comprises SEQ ID NO: 6. In some embodiments, the endogenous miRNA sequence is at least 80% identical to SEQ ID NO: 6. In some embodiments, the endogenous miRNA sequence is about 80% identical to SEQ ID NO: 6. In some embodiments, the endogenous miRNA sequence is about 81% identical to SEQ ID NO: 6. In some embodiments, the endogenous miRNA sequence is about 82% identical to SEQ ID NO: 6. In some embodiments, the endogenous miRNA sequence is about 83% identical to SEQ ID NO: 6. In some embodiments, the endogenous miRNA sequence is about 84% identical to SEQ ID NO: 6. In some embodiments, the endogenous miRNA sequence is about 85% identical to SEQ ID NO: 6. In some embodiments, the endogenous miRNA sequence is about 86% identical to SEQ ID NO: 6. In some embodiments, the endogenous miRNA sequence is about 87% identical to SEQ ID NO: 6. In some embodiments, the endogenous miRNA sequence is about 88% identical to SEQ ID NO: 6. In some embodiments, the endogenous miRNA sequence is about 89% identical to SEQ ID NO: 6. In some embodiments, the endogenous miRNA sequence is about 90% identical to SEQ ID NO: 6. In some embodiments, the endogenous miRNA sequence is about 91% identical to SEQ ID NO: 6. In some embodiments, the endogenous miRNA sequence is about 92% identical to SEQ ID NO: 6. In some embodiments, the endogenous miRNA sequence is about 93% identical to SEQ ID NO: 6. In some embodiments, the endogenous miRNA sequence is about 94% identical to SEQ ID NO: 6. In some embodiments, the endogenous miRNA sequence is about 95% identical to SEQ ID NO: 6. In some embodiments, the endogenous miRNA sequence is about 96% identical to SEQ ID NO: 6. In some embodiments, the endogenous miRNA sequence is about 97% identical to SEQ ID NO: 6. In some embodiments, the endogenous miRNA sequence is about 98% identical to SEQ ID NO: 6. In some embodiments, the endogenous miRNA sequence is about 99% identical to SEQ ID NO: 6. In some embodiments, the nucleic acid comprises one or more target segment(s) of any of the above miRNAs.

In some embodiments, the nucleic acid comprises one or more repeat(s) of a target segment comprising a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 7. In some specific embodiments, the nucleic acid comprises one or more repeat(s) of the target segment having SEQ ID NO: 7. In some embodiments, the nucleic acid comprises 1, 2, 3, 4, 5, 6, 7, 8, 9, 10 or more repeats of any of the above target segment(s), e.g., the target segment having a nucleic acid sequence of SEQ ID NO: 7.

In some embodiments, the nucleic acid comprises one or more repeat(s) of a target segment comprising a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 8. In some specific embodiments, the nucleic acid comprises one or more repeat(s) of the target segment having SEQ ID NO: 8. In some embodiments, the nucleic acid comprises 1, 2, 3, 4, 5, 6, 7, 8, 9, 10 or more repeats of any of the above target segment(s), e.g., the target segment having a nucleic acid sequence of SEQ ID NO: 8.

In some embodiments, the nucleic acid comprises one or more repeat(s) of a target segment comprising a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 9. In some specific embodiments, the nucleic acid comprises one or more repeat(s) of the target segment having SEQ ID NO: 9. In some embodiments, the nucleic acid comprises 1, 2, 3, 4, 5, 6, 7, 8, 9, 10 or more repeats of any of the above target segment(s), e.g., the target segment having a nucleic acid sequence of SEQ ID NO: 9.

In some embodiments, the nucleic acid comprises one or more repeat(s) of a target segment comprising a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 10. In some specific embodiments, the nucleic acid comprises one or more repeat(s) of the target segment having SEQ ID NO: 10. In some embodiments, the nucleic acid comprises 1, 2, 3, 4, 5, 6, 7, 8, 9, 10 or more repeats of any of the above target segment(s), e.g., the target segment having a nucleic acid sequence of SEQ ID NO: 10.

In some embodiments, the nucleic acid comprises one or more repeat(s) of a target segment comprising a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 11. In some specific embodiments, the nucleic acid comprises one or more repeat(s) of the target segment having SEQ ID NO: 11. In some embodiments, the nucleic acid comprises 1, 2, 3, 4, 5, 6, 7, 8, 9, 10 or more repeats of any of the above target segment(s), e.g., the target segment having a nucleic acid sequence of SEQ ID NO: 11.

In some embodiments, the nucleic acid comprises one or more repeat(s) of a target segment comprising a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 12. In some specific embodiments, the nucleic acid comprises one or more repeat(s) of the target segment having SEQ ID NO: 12. In some embodiments, the nucleic acid comprises 1, 2, 3, 4, 5, 6, 7, 8, 9, 10 or more repeats of any of the above target segment(s), e.g., the target segment having a nucleic acid sequence of SEQ ID NO: 12.

In some embodiments, the nucleic acid comprises at least one target segment of hsa-mir-133a (or hsa-mir-133a-1). In some embodiments, the nucleic acid comprises at least 2 repeats of a target segment of hsa-mir-133a (or hsa-mir-133a-1). In some embodiments, the nucleic acid comprises at least 3 repeats of a target segment of hsa-mir-133a (or hsa-mir-133a-1).

In some embodiments, the nucleic acid comprises at least one target segment of hsa-mir-133a (or hsa-mir-133a-1) and at least one target segment of a liver specific miRNA. In some embodiments, the nucleic acid comprises at least 2 repeats of a target segment of hsa-mir-133a (or hsa-mir-133a-1) and at least one target segment of a liver specific miRNA. In some embodiments, the nucleic acid comprises at least 3 repeats of a target segment of hsa-mir-133a (or hsa-mir-133a-1) and at least one target segment of a liver specific miRNA. In some embodiments, the liver specific miRNA is hsa-mir-122. In some more specific embodiments, the target segment of hsa-mir-133a comprises SEQ ID NO: 11 and the target segment of hsa-mir-122 comprises SEQ ID NO: 10.

TABLE 3
Exemplary Linkers

| Linker name | Linker sequence |
|---|---|
| EXG-Link01 | 5′-cttgac-3′ (SEQ ID NO: 13) |
| EXG-Link02 | 5′-ccatag-3′ (SEQ ID NO: 14) |
| EXG-Link03 | 5′-tttcta-3′ (SEQ ID NO: 15) |
| EXG-Link04 | 5′-caagct-3′ (SEQ ID NO: 16) |
| EXG-Link05 | 5′-gatcta-3′ (SEQ ID NO: 17) |

In some embodiments, multiple target segments within a target sequence in the present nucleic acid constructs may be overlapping. Alternatively, they may be non-overlapping. In some embodiments, target segments within a target region are separated by a number of nucleotides that is, is about, is no more than, is no more than about, 250, 200, 150, 100, 90, 80, 70, 60, 50, 40, 30, 20, or 10 nucleotides, or is a range defined by any two of the preceding values. In some embodiments, target segments within a target sequence are separated by no more than, or no more than about, 5 nucleotides. According to some embodiments, target segments are contiguous.

In some embodiments, one or more linkers are present between two target segments. In some embodiments, all target segments are linked by linkers. In other embodiments, only part of the target segments are linked by linkers. In some embodiments, the linkers in the nucleic acid are the same. In other embodiments, different linkers are in the present nucleic acid.

Exemplary linkers are shown in Table 3. In some embodiments, the nucleic acid comprises one or more linker(s) having SEQ ID NO: 13. In some embodiments, the nucleic acid comprises one or more linker(s) having SEQ ID NO: 14. In some embodiments, the nucleic acid comprises one or more linker(s) having SEQ ID NO: 15. In some embodiments, the nucleic acid comprises one or more linker(s) having SEQ ID NO: 16. In some embodiments, the nucleic acid comprises one or more linker(s) having SEQ ID NO: 17. In some embodiments, the nucleic acid comprises two or more linkers each independently selected from SEQ ID NO: 13, SEQ ID NO: 14, SEQ ID NO: 15, SEQ ID NO: 16, and SEQ ID NO: 17.

Exemplary nucleic acids (or target sequences of miRNAs) comprising multiple target segments of multiple endogenous miRNAs are shown in Table 6 in Section 6 below.

In some embodiments, the nucleic acid (or a target sequences of miRNAs) comprising multiple target segments comprises at least one target segment of hsa-mir-208a, at least one target segment of hsa-mir-208b, at least one target segment of hsa-mir-122, and at least one target segment of hsa-mir-133a. In some more specific embodiments, the nucleic acid (or a target sequences of miRNAs) comprising multiple target segments comprises 2 target segments of hsa-mir-208a, 2 target segments of hsa-mir-208b, 3 target segments of hsa-mir-122, and 3 target segments of hsa-mir-133a. In a specific embodiment, the nucleic acid (or a target sequences of miRNAs) comprising multiple target segments comprises 2 repeats of a target segment having a nucleic acid sequence of SEQ ID NO: 8, 2 repeats of a target segment having a nucleic acid sequence of SEQ ID NO: 9, 3 repeats of a target segment having a nucleic acid sequence of SEQ ID NO: 10, and 3 repeats of a target segment having a nucleic acid sequence of SEQ ID NO: 11. In some embodiments, the nucleic acid comprising multiple target segments (or a target sequences of miRNAs) comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 19.

In some embodiments, the nucleic acid comprising multiple target segments (or a target sequences of miRNAs) comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 18.

In some embodiments, the nucleic acid comprising multiple target segments (or a target sequences of miRNAs) comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 20.

In some embodiments, the nucleic acid comprising multiple target segments (or a target sequences of miRNAs) comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 21.

In some embodiments, the component target segments are in the same order of those in SEQ ID NO: 18, SEQ ID NO: 19, SEQ ID NO; 20, or SEQ ID NO: 21. In other embodiments, the component target segments are in a different order (from 5′ to 3′) from those in SEQ ID NO: 18, SEQ ID NO: 19, SEQ ID NO; 20, or SEQ ID NO: 21.

5.2.3. Synthetic Promoters

As demonstrated in Section 6 below, certain synthetic promoters provided herein provide surprisingly superior effects, particularly in use in an AAV based gene therapy for delivering SMN.

Thus, in yet another aspect, provided herein is a nucleic acid comprising a nucleic acid region comprising a nucleic acid sequence encoding a SMN protein or variant thereof and a synthetic promoter comprising an enhancer and a core promoter.

In some embodiments, the synthetic promoter comprises CMV enhancer and hSyn promoter. In some embodiments, the synthetic promoter comprises proC3 enhancer and hSyn promoter. In some embodiments, the synthetic promoter comprises proA5 enhancer and hSyn promoter. In other embodiments, the synthetic promoter comprises proB15 enhancer and hSyn promoter.

In some embodiments, the synthetic promoter comprises CMV enhancer and hSyn promoter, wherein the hSyn promoter comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 38, and the CMV enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 39.

In some embodiments, the synthetic promoter comprises proC3 enhancer and hSyn promoter, wherein the hSyn promoter comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 38, and the proC3 enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 40.

In some embodiments, the synthetic promoter comprises proA5 enhancer and hSyn promoter, wherein the hSyn promoter comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 38, and the proA5 enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 41.

In some embodiments, the synthetic promoter comprises proB15 enhancer and hSyn promoter, wherein the hSyn promoter comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 38, the proB15 enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 42.

In some embodiments, the SMN protein or variant thereof comprises an amino acid sequence of SEQ ID NO: 33, or an amino acid sequence having at least 80%, 85%, 90%, 91%, 92%, 93%, 94%, 95%, 96%, 97%, 98%, 99% identity to SEQ ID NO: 33.

In some embodiments, the nucleic acid sequence encoding the SMN protein or variant thereof comprises a nucleic acid sequence selected from a group consisting of SEQ ID NO: 34 and SEQ ID NO: 35.

Exemplary rAAV vectors comprising the present synthetic promoters are described in Section 6 below.

In some embodiments, provided herein is a rAAV vector comprising a nucleic acid sequence of SEQ ID NO: 45, or a nucleic acid sequence having at least 80%, 85%, 90%, 91%, 92%, 93%, 94%, 95%, 96%, 97%, 98%, 99% identify to SEQ ID NO: 45.

In some embodiments, provided herein is a rAAV vector comprising a nucleic acid sequence of SEQ ID NO: 46, or a nucleic acid sequence having at least 80%, 85%, 90%, 91%, 92%, 93%, 94%, 95%, 96%, 97%, 98%, 99% identify to SEQ ID NO: 46.

In some embodiments, provided herein is a rAAV vector comprising a nucleic acid sequence of SEQ ID NO: 47, or a nucleic acid sequence having at least 80%, 85%, 90%, 91%, 92%, 93%, 94%, 95%, 96%, 97%, 98%, 99% identify to SEQ ID NO: 47.

In some embodiments, provided herein is a rAAV vector comprising a nucleic acid sequence of SEQ ID NO: 48, or a nucleic acid sequence having at least 80%, 85%, 90%, 91%, 92%, 93%, 94%, 95%, 96%, 97%, 98%, 99% identify to SEQ ID NO: 48.

In some embodiments, provided herein is a rAAV vector comprising a nucleic acid sequence of SEQ ID NO: 49, or a nucleic acid sequence having at least 80%, 85%, 90%, 91%, 92%, 93%, 94%, 95%, 96%, 97%, 98%, 99% identify to SEQ ID NO: 49.

In some embodiments, provided herein is a rAAV vector comprising a nucleic acid sequence of SEQ ID NO: 50, or a nucleic acid sequence having at least 80%, 85%, 90%, 91%, 92%, 93%, 94%, 95%, 96%, 97%, 98%, 99% identify to SEQ ID NO: 50.

In some embodiments, provided herein is a rAAV vector comprising a nucleic acid sequence of SEQ ID NO: 51, or a nucleic acid sequence having at least 80%, 85%, 90%, 91%, 92%, 93%, 94%, 95%, 96%, 97%, 98%, 99% identify to SEQ ID NO: 51.

In some embodiments, provided herein is a rAAV vector comprising a nucleic acid sequence of SEQ ID NO: 52, or a nucleic acid sequence having at least 80%, 85%, 90%, 91%, 92%, 93%, 94%, 95%, 96%, 97%, 98%, 99% identify to SEQ ID NO: 52.

In some embodiments, provided herein is a rAAV vector comprising a nucleic acid sequence of SEQ ID NO: 53, or a nucleic acid sequence having at least 80%, 85%, 90%, 91%, 92%, 93%, 94%, 95%, 96%, 97%, 98%, 99% identify to SEQ ID NO: 53.

### 5.3. Recombinant Viral Vectors and Viral Particles for Gene Therapies

Also provided herein are virus based gene therapies for delivering the nucleic acid provided herein. Thus, in another aspect, provided herein are viral vectors (e.g., rAAV vectors) comprising the nucleic acid (comprising a nucleic acid region encoding a protein of interest and a nucleic acid region comprising target segments of miRNAs) provided herein. In yet another aspect, provided herein are viral particles (e.g., rAAVs or rAAV particles) comprising the nucleic acid provided herein.

5.3.1. Virus Based Gene Delivery Systems

A number of viral based systems have been developed for gene transfer into mammalian cells. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

In certain embodiments, the viral vector or viral particle provided herein is derived from an adenovirus. Exemplary vectors are based on or derived from HAd5, ChAd3, HAd26, HAd6, AdCH3NSmut, HAd35, ChAd63, HAd4, rcAd26. Recombinant adenovirus vectors can be constructed according to known methods in the art. See, e.g., O'Connor et al., Virology, 217(1):11-22 (1996); Hardy et al., Journal of Virology, 73(9):7835-7841 (1999); Hardy et al., Journal of Virology, 71(3):1842-1849 (1997). In some embodiments, third-generation adenoviral vectors (also called “high capacity adenoviral vectors” (HCAds), helper-dependent or “gutless” adenoviral vectors) can be used herein to deliver longer sequences. In some embodiments, the polynucleotide of interest, e.g., a transgene is cloned into an adenoviral vector that only contains the ITRs and a packaging signal. A helper adenoviral vector may be co-transfected into HEK cells to generate the adenoviral particle. See Lee et al., Genes and Diseases, 4(2):43-63 (2007).

In certain embodiments, the viral vector or viral particle provided herein is derived from a lentivirus. Exemplary vectors are based on or derived from HIV-1, HIV-2, SIVSM, SIVAGM, EIAV, FIV, VNV, CAEV, or BIV. Lentiviral vectors can be produced according to the known methods in the art, e.g., as described in Cribbs et al., BMC Biotechnology, 13:98 (2003); Merten et al., Mol Ther Methods Clin Dev., 13 (3):16017 (2016); Durand and Cimarelli, Viruses, 3:132-159 (2011). In some embodiments, third-generation self-inactivating lentiviral vectors are used herein.

In certain embodiments, the viral vector or viral particle provided herein is derived from a herpes simplex virus (HSV). In some embodiments, the herpes simplex virus is a herpes simplex type 1 virus (HSV-1), a herpes simplex type 2 virus (HSV-2), or any derivative thereof. Exemplary vectors are based on or derived from HSV-1, HSV-2, CMV, VZV, EBV, and KSHV. HSV-based vectors can be constructed according the methods known in the art, e.g., as described in U.S. Pat. Nos. 7,078,029, 6,261,552, 5,998,174, 5,879,934, 5,849,572, 5,849,571, 5,837,532, 5,804,413, and 5,658,724, and International Patent Applications WO 91/02788, WO 96/04394, WO 98/15637, and WO 99/06583, which are incorporated herein by reference in their entireties.

In some embodiments, the HSV-based vector provided herein is an amplicon vector. In other embodiments, the HSV-based vector provided herein is a replication-defective vector. In yet other embodiments, the HSV-based vector provided herein is a replication-competent vector.

The amplicons are plasmid-derived vectors engineered to contain both the origin of HSV DNA replication (ori) and HSV cleavage-packaging recognition sequences (pac). When amplicons are transfected into mammalian cells with HSV helper functions, they are replicated, form head-to-tail linked concatamers and are then packaged into viral particles. There are two major methods currently used for producing amplicon particles, one based on infection with defective helper HSVs and the other based on transfection of HSV-1 genes, such as a set of pac-deleted overlapping cosmids or a pac-deleted and ICP27-deleted BAC-HSV-1. In some embodiments, amplicons used herein can accommodate large fragments of foreign DNA (e.g., up to 152 kb), including multiple copies of the transgene (e.g., up to 15 copies), and are non-toxic.

In some embodiments, an HSV-based vector used herein is deficient in at least one essential HSV gene, and the HSV-based vector may also comprise one or more deletions of non-essential genes. In some embodiments, the HSV-based vector is replication-deficient. Most replication-deficient HSV-based vectors contain a deletion to remove one or more intermediate-early, early, or late HSV genes to prevent replication. In other embodiments, the HSV-based vector is deficient in an immediate early gene selected from the group consisting of ICP0, ICP4, ICP22, ICP27, ICP47, and a combination thereof. In a specific embodiment, the HSV-based vector is deficient for all of ICP0, ICP4, ICP22, ICP27, and ICP47. Exemplary replication-competent vectors include NV-1020 (HSV-1), RAV9395 (HSV-2), AD-472 (HSV-2), NS-gEnull (HSV-1), and ImmunoVEX (HSV2). Exemplary replication-defective vectors include dl5-29 (HSV-2), dl5-29-41L (HSV-1), DISC-dH (HSV-1 and HSV-2), CJ9gD (HSV-1), TOH-OVA (HSV-1), d106 (HSV-1), d81 (HSV-1), HSV-SIV d106 (HSV-1), and d106 (HSV-1).

Replication-deficient HSV-based vectors are typically produced in complementing cell lines that provide gene functions not present in the replication-deficient HSV-based vectors, but required for viral propagation, at appropriate levels in order to generate high titers of viral vector stock. An exemplary cell line complements for at least one and, in some embodiments, all replication-essential gene functions not present in a replication-deficient HSV-based vector. For example, a HSV-based vector deficient in ICP0, ICP4, ICP22, ICP27, and ICP47 can be complemented by the human osteosarcoma line U2OS. The cell line can also complement non-essential genes that, when missing, reduce growth or replication efficiency (e.g., UL55). The complementing cell line can complement for a deficiency in at least one replication-essential gene function encoded by the early regions, immediate-early regions, late regions, viral packaging regions, virus-associated regions, or combinations thereof, including all HSV functions (e.g., to enable propagation of HSV amplicons, which comprise minimal HSV sequences, such as only inverted terminal repeats and the packaging signal or only ITRs and an HSV promoter). In some embodiments, the cell line is further characterized in that it contains the complementing genes in a non-overlapping fashion with the HSV-based vector, which minimizes, and practically eliminates, the possibility of the HSV-based vector genome recombining with the cellular DNA. Accordingly, the presence of replication competent HSV is minimized, if not avoided in the vector stock, which, therefore, is suitable for certain therapeutic purposes, especially gene therapy purposes. The construction of complementing cell lines involves standard molecular biology and cell culture techniques well known in the art.

In certain embodiments, the viral vector or viral particle provided herein is derived from an adeno-associated virus (AAV). More detailed description related to AAV is provided in Sections 5.3.2-5.3.4 below.

The nucleic acid of interest can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present disclosure. Promoters may be roughly categorized as constitutive promoters or regulated promoters, such as inducible promoters.

In some embodiments, the nucleic acid provided herein is operably linked to a constitutive promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1 alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies.

In some embodiments, the nucleic acid provided herein is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof.

In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

A person skilled in the art may recognize that a target cell may require a specific promoter including but not limited to a promoter that is species specific, inducible, tissue-specific, or cell cycle-specific Parr et al., Nat. Med. 3:1145-9 (1997); the contents of which are herein incorporated by reference in its entirety. In one embodiment, the promoter is a promoter deemed to be efficient to drive the expression of the polynucleotides described herein. Promoters for which promote expression in most tissues include, for example, but are not limited to, human elongation factor 1α-subunit (EF1α), immediate-early cytomegalovirus (CMV), the RSV LTR, the MoMLV LTR, the phosphoglycerate kinase-1 (PGK) promoter, a simian virus 40 (SV40) promoter and a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), the telomerase (hTERT) promoter, chicken β-actin (CBA) and its derivative CAG and miniCBA, the β glucuronidase (GUSB), or ubiquitin C (UBC). Tissue-specific expression elements can be used to restrict expression to certain cell types such as, but not limited to, nervous system promoters which can be used to restrict expression to neurons, astrocytes, or oligodendrocytes. Non-limiting example of tissue-specific expression elements for neurons include neuron-specific enolase (NSE), platelet-derived growth factor (PDGF), platelet-derived growth factor B-chain (PDGF-β), the synapsin (Syn), the methyl-CpG binding protein 2 (MeCP2), CaMKII, mGluR2, NFL, NFH, nβ2, PPE, Enk and EAAT2 promoters. The above metioned promoters can be combined with other synthetic short regulatory elements to generate novel synthetic promoters with center homology in DNA sequences.

In some embodiments, the promoter is capable of expressing the heterologous nucleic acid in a neuronal cell. In some embodiments, the promoter is capable of expressing the heterologous nucleic acid in a motor neuron cell. In some embodiments, the promoter is capable of expressing the heterologous nucleic acid in astrocytes. According to some embodiments, the promoter is a human Synapsin 1 (hSyn) promoter, or hSyn combined with synthetic regulatory element that is specific for neuronal cells. According to some embodiments, the promoter is a glial fibrillary acidic protein (GFAP) or EAAT2 promoter or GFAP or EAAT2 combined with synthetic regulatory element, that are specific for astrocytes.

In one embodiment, the nucleic acid construct comprises a promoter such as, but not limited to, CMV or U6, or CMV or U6 combined with synthetic regulatory element. As a non-limiting example, the promoter in the present rAAV vector is a CBA or a miniCBA promoter. As another non-limiting example, the promoter in the present rAAV vector is a modified miniCBA promoter. In one embodiment, the rAAV vector has an engineered promoter. In one embodiment, the rAAV vector further comprises a synthetic enhancer element.

In one embodiment, the vector genome comprises at least one element to enhance the transgene target specificity and expression (See e.g., Powell et al. Viral Expression Cassette Elements to Enhance Transgene Target Specificity and Expression in Gene Therapy, 2015; the contents of which are herein incorporated by reference in its entirety) such as an intron, or a synthetic intron with modified sequences from mammalian genomes. Non-limiting examples of introns include, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps), and hybrid adenovirus splice donor/IgG splice acceptor (230 bps). In one embodiment, the intron may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The promoter may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

In some embodiments, the vector also contains a selectable marker gene or a reporter gene to select cells expressing the protein from the population of host cells transfected through vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

5.3.2. Recombinant AAV Vectors

In certain more specific embodiments, the nucleic acid provided herein is delivered by a AAV based system, and thus is included in a recombinant AAV vector.

Any AAV serotypes or variants thereof can be used in the present disclosure. AAV serotypes may include, but not limited to, AAV1 (Genbank Accession No. NC_002077.1; HC000057.1), AAV2 (Genbank Accession No. NC_001401.2, JC527779.1), AAV2i8 (Asokan, A., 2010, Discov. Med. 9:399), AAV3 (Genbank Accession No. NC_001729.1), AAV3-B (Genbank Accession No. AF028705.1), AAV4 (Genbank Accession No. NC_001829.1), AAV5 (Genbank Accession No. NC_006152.1; JC527780.1), AAV6 (Genbank Accession No. AF028704.1; JC527781.1), AAV7 (Genbank Accession No. NC_006260.1; JC527782.1), AAV8 (Genbank Accession No. NC_006261.1; JC527783.1), AAV9 (Genbank Accession No AX753250.1; JC527784.1), AAV10 (Genbank Accession No AY631965.1), AAVrh10 (Genbank Accession No. AY243015.1), AAV11 (Genbank Accession No AY631966.1), AAV12 (Genbank Accession No DQ813647.1), AAV13 (Genbank Accession No EU285562.1), AAV LK03, AAVrh74, AAV DJ (Wu Z, et al., J Virol. 80:11393-7 (2006)), AAVAnc81, Anc82, Anc83, Anc84, Anc110, Anc113, Anc126, or Anc127 (Zin, E. et al., Cell. Rep. 12:1056 (2016)), AAV_go.1 (Arbetum, A. E. et al., J. Virol. 79:15238 (2005)), AAVhu.37, AAVrh8, AAVrh8R, and AAV rh.8 (Wang et al., Mol. Ther. 18:119-125 (2010), or variants thereof.

AAV variants include, but not limited to, AAV1 variants (e.g., AAV comprising AAV1 variant capsid proteins), AAV2 variants (e.g., AAV comprising AAV2 variant capsid proteins), AAV3 variants (e.g., AAV comprising AAV3 variant capsid proteins), AAV3-B variants (e.g., AAV comprising AAV3-B variant capsid proteins), AAV4 variants (e.g., AAV comprising AAV4 variant capsid proteins), AAV5 variants (e.g., AAV comprising AAV5 variant capsid proteins), AAV6 variants (e.g., AAV comprising AAV6 variant capsid proteins), AAV7 variants (e.g., AAV comprising AAV7 variant capsid proteins), AAV8 variants (e.g., AAV comprising AAV8 variant capsid proteins), AAVrh8, AAVrh8R (e.g., AAV comprising AAVrh8 or AAVrh8R variant capsid proteins), AAV9 variants (e.g., AAV comprising AAV9 variant capsid proteins), AAV10 variants (e.g., AAV comprising AAV10 variant capsid proteins), AAVrh10 variants (e.g., AAV comprising AAVrh10 variant capsid proteins), AAV11 variants (e.g., AAV comprising AAV11 variant capsid proteins), AAV12 variants (e.g., AAV comprising AAV12 variant capsid proteins), AAV13 variants (e.g., AAV comprising AAV13 variant capsid proteins), AAV LK03 variants (e.g., AAV comprising AAV LK03 variant capsid proteins), or AAVrh74 variants (e.g., AAV comprising AAVrh74 variant capsid proteins).

Recombinant AAV (rAAV) vectors used in the present disclosure can be constructed according to known techniques. In some embodiments, the rAAV vector is constructed to include operatively linked components in the direction of transcription, control dements including a transcriptional initiation region, the polynucleotide provided herein and a transcriptional termination region. The control elements can be selected based on the cell of interest. In some embodiments, the resulting rAAV vector construct comprising the operatively linked components is franked (5′ and 3′) with functional AAV FIR sequences.

In certain embodiments, the polypeptide (e.g., encoding a SMN protein) provided herein is operatively linked to at least one regulatory sequence. In certain embodiments, regulatory sequences may, for example, include promoter sequences, enhancer sequences, e.g., upstream enhancer sequences (USEs), RNA processing signals, e.g., splicing signals, polyadenylation signal sequences, sequences that stabilize cytoplasmic mRNA, post-transcriptional regulatory elements (PREs) and/or microRNA (miRNA) target sequences. In certain embodiments, regulatory sequences may include sequences that enhance translation efficiency (e.g., Kozak sequences), sequences that enhance protein stability, and/or sequences that enhance protein processing and/or secretion. In certain embodiments, the polynucleotide may encode regulatory miRNAs.

In certain embodiments, a regulatory sequence comprises a constitutive promoter and/or regulatory control element. In certain embodiments, a regulatory sequence comprises a regulatable promoter and/or regulatory control element. In certain embodiments, a regulatory sequence comprises a ubiquitous promoter and/or regulatory control element. In certain embodiments, a regulatory sequence comprises a cell- or tissue-specific promoter and/or regulatory control element. In certain embodiments, the regulatory control element is 5′ of the coding sequence of the protein (that is, is present in ′5 untranslated regions; 5′ UTRs). In other embodiments, the regulatory control element is 3′ of the coding sequence of the protein (that is, is present in ′3 untranslated regions; 3′ UTRs). In certain embodiments, the polynucleotide comprises more than one regulatory control element, for example may comprise two, three, four or five control elements. In instances wherein the polynucleotide comprises more than one control element, each control element may independently be 5′ of, 3′ of, flank, or within the coding sequence of the protein.

In certain embodiments, the control element is an enhancer. In some embodiments, the control elements included direct the transcription or expression of the polynucleotide of the protein in the subject in vivo. Control elements can comprise control sequences normally associated with the selected polynucleotide of interest or alternatively heterologous control sequences.

Exemplary control sequences include those derived from sequences encoding mammalian or viral genes, such as neuron-specific enolase promoter, a GFAP promoter, the SV40 early promoter, mouse mammary tumor virus LTR promoter, adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, and hybrid promoters.

In certain embodiments, a promoter is not cell- or tissue-specific, e.g., the promoter is considered a ubiquitous promoter. Examples of promoter sequences that may promote expression in multiple cell or tissue types include, for example, human elongation factor 1a-subunit (EF1a), cytomegalovirus (CMV) immediate-early enhancer and/or promoter, chicken beta-actin (CBA) and its derivatives, e.g., CAG, for example, a CBA promoter with an S40 intron, beta glucuronidase (GUSB), or ubiquitin C (UBC).

In certain embodiments, a promoter sequence can promote expression in particular cell types or tissues. For example, in certain embodiments, a promoter may be a muscle-specific promoter, e.g., may be a mammalian muscle creatine kinase (MCK) promoter, mammalian desmin (DES) promoter, mammalian troponin I (TNNI2) promoter, or a mammalian skeletal alpha-actin (ASKA) promoter. In other embodiments, a promoter sequence may be able to promote expression in neural cells or cell types, e.g., may be a neuron-specific enolase (NSE), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), beta-globin minigene hb2, preproenkephalin (PPE), enkephalin (Enk) or excitatory amino acid transporter 2 (EAAT2) promoter. In other embodiments, a promoter sequence may promote expression in the liver, e.g., may be an alpha-1-antitrypsin (hAAT) or thyroxine binding globulin (TBG) promoter. In yet other embodiments, a promoter sequence may promote expression in cardiac tissue, e.g., may be a cardiomyocyte-specific promoter such as an MHC, cTnT, or CMV-MUC2k promoter.

In certain embodiments, the polynucleotide may comprise at least one polyadenylation (polyA) signal sequence, which are well known in the art. In instances where a polyadenylation sequence is present, it is generally located between the 3′ end of the transgene coding sequence and the 5′ end of the 3′ ITR. In certain embodiments, the polynucleotide further comprises a polyA upstream enhancer sequence 5′ of the polyA signal sequence. In certain instances, the regulatory sequence is a sequence that increases translation efficiency, e.g., a Kozak sequence.

In certain embodiments, the polynucleotide comprises an intron. In certain embodiments, the intron is present within the coding sequence of the protein provided herein. In certain embodiments, the intron is 5′ or 3′ of the coding sequence of the protein. In certain embodiments, the intron flanks the 5′ or 3′ terminus of the coding sequence of the protein. In certain embodiments, the polynucleotide comprises two introns. In some embodiments, one intron is 5′ of and one intron is 3′ of the coding sequence of the protein. In certain embodiments, one intron flanks the 5′ terminus of the coding sequence of the protein and the second intron flanks the 3′ terminus of the coding sequence of the protein. In certain embodiments, the intron is an SV40 intron, e.g., a 5′ UTR SV40 intron.

The sequences of AAV ITR known in the art can be used in the present rAAV vector. In some embodiments, the AAV ITR used in the present vectors has a wild-type nucleotide sequence. In other embodiments, the AAV ITR sequence used in the present vectors is not wild-type sequence, and instead it comprises, e.g., the insertion, deletion or substitution of nucleotides. AAV ITRs provided herein may be derived from any AAV serotypes, including but not limited to, AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV LK03, AAVrh74, AAV44-9, or a variant thereof.

In some embodiments, the 5′ and 3′ ITRs which flank a nucleotide sequence in a rAAV vector provided herein are identical and derived from the same AAV serotype. In other embodiments, the 5′ and 3′ LTRs which flank a nucleotide sequence in a rAAV vector provided herein are different and/or derived from different AAV serotypes.

In some embodiments, the rAAV vector comprising the polynucleotide of the protein flanked by AAV ITRs can be constructed by directly inserting the polynucleotide of interest into are AAV genome, e.g., into an excised AAV open reading frames, and certain portions of the AAV genome can optionally be deleted, as described in, e.g., WO 1993/003769; Kotin (1994) Human Gene Therapy 5:793-801; Shelling and Smith (1994) Gene Therapy 1:165469; and Zhou et al. (1994) J. Exp. Med. 179:1867-1875.

In other embodiments, AAV ITRs are excised from an AAV genome or from an AAV vector containing such ITRs, and then are fused to 5′ and 3′ of a polynucleotide sequence of the protein that is present in another vector using standard ligation techniques.

In certain embodiments, the rAAV vector provided herein comprises a recombinant self-complementing genome. A rAAV comprising a self-complementing genome can usually quickly form a double stranded DNA molecule by its partially complementing sequences (e.g., complementing coding and non-coding strands of a transgene). More specifically, in some embodiments, an rAAV vector provided herein comprises an rAAV genome that comprises a first heterologous polynucleotide sequence (e.g., a therapeutic transgene coding strand) and a second heterologous polynucleotide sequence (e.g., the noncoding or antisense strand of the therapeutic transgene), and the first heterologous polynucleotide sequence can form intrastrand base pairs with the second polynucleotide sequence. In some embodiments, the first heterologous polynucleotide sequence and a second heterologous polynucleotide sequence are linked by a sequence that facilitates intrastrand base-pairing, e.g., a hairpin DNA structure. In some embodiments, the first heterologous polynucleotide sequence and a second heterologous polynucleotide sequence are linked by a mutated ITR, so that the rep proteins do not cleave the viral genome at the mutated ITR. rAAV vectors comprising self-complementing genomes can be made using the methods known in the art, e.g., as described in U.S. Pat. Nos. 7,125,717; 7,785,888; 7,790,154; 7,846,729; 8,093,054; and 8,361,457.

In some embodiments, the polynucleotide molecules in the rAAV vectors provided herein is less than about 5 kilobases (kb) in size. In some embodiments, the polynucleotide molecules in the rAAV vectors provided herein is less than about 4.5 kb in size. In some embodiments, the polynucleotide molecules in the rAAV vectors provided herein is less than about 4.0 kb in size. In some embodiments, the polynucleotide molecules in the rAAV vectors provided herein is less than about 3.5 kb in size. In some embodiments, the polynucleotide molecules in the rAAV vectors provided herein is less than about 3.0 kb in size. In some embodiments, the polynucleotide molecules in the rAAV vectors provided herein is less than about 2.5 kb in size.

In one aspect, provided herein is a recombinant AAV (rAAV) vector comprising (i) a first nucleic acid region comprising a transgene; and (ii) a second nucleic acid region comprising one or more target segment(s) of one or more endogenous miRNA(s), wherein at least one target segment is a target segment of an endogenous miRNA in heart, and at least one target segment is a target segment of an endogenous miRNA in liver; wherein the second nucleic acid region is at 3′ of the first nucleic acid region; and wherein the rAAV vector comprises an inverted terminal repeat (ITR) from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, or AAV44-9. In some embodiment, the first nucleic acid encodes SMN or a variant thereof.

In some specific embodiments, the second nucleic acid region in the present rAAV vector comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 18. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11 In some embodiments, the rAAV vector comprises an ITR from AAV12. In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the second nucleic acid region in the present rAAV vector comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 19. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4. In some embodiments, the rAAV vector comprises an ITR from AAV5 In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12. In some embodiments, the rAAV vector comprises an ITR from AAV13 In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the second nucleic acid region in the present rAAV vector comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 20. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AA w′3-B. In some embodiments, the rAAV vector comprises an ITR from AAV4. In some embodiments, the rAAV vector comprises an ITR from AAV5. In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12. In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh74.

In some specific embodiments, the second nucleic acid region in the present rAAV vector comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 21. In some embodiments, the rAAV vector comprises an ITR from AAV1. In some embodiments, the rAAV vector comprises an ITR from AAV2. In some embodiments, the rAAV vector comprises an ITR from AAV2i8. In some embodiments, the rAAV vector comprises an ITR from AAV3. In some embodiments, the rAAV vector comprises an ITR from AAV3-B. In some embodiments, the rAAV vector comprises an ITR from AAV4. In some embodiments, the rAAV vector comprises an ITR from AAV5 In some embodiments, the rAAV vector comprises an ITR from AAV6. In some embodiments, the rAAV vector comprises an ITR from AAV7. In some embodiments, the rAAV vector comprises an ITR from AAV8. In some embodiments, the rAAV vector comprises an ITR from AAVrh8. In some embodiments, the rAAV vector comprises an ITR from AAVrh8R. In some embodiments, the rAAV vector comprises an ITR from AAV9. In some embodiments, the rAAV vector comprises an ITR from AAV10. In some embodiments, the rAAV vector comprises an ITR from AAVrh10. In some embodiments, the rAAV vector comprises an ITR from AAV11. In some embodiments, the rAAV vector comprises an ITR from AAV12. In some embodiments, the rAAV vector comprises an ITR from AAV13. In some embodiments, the rAAV vector comprises an ITR from AAV-DJ. In some embodiments, the rAAV vector comprises an ITR from AAV LK03. In some embodiments, the rAAV vector comprises an ITR from AAVrh4.

In some more specific embodiments, provided herein is a vector comprising a nucleic acid sequence of SEQ ID NO: 22, or a nucleic acid sequence having at least 80%, 85%, 90%, 91%, 92%, 93%, 94%, 95%, 96%, 97%, 98%, 99% identify to SEQ ID NO: 22.

In some more specific embodiments, provided herein is a vector comprising a nucleic acid sequence of SEQ ID NO: 23, or a nucleic acid sequence having at least 80%, 85%, 90%, 91%, 92%, 93%, 94%, 95%, 96%, 97%, 98%, 99% identify to SEQ ID NO: 23.

In some more specific embodiments, provided herein is a vector comprising a nucleic acid sequence of SEQ ID NO: 24, or a nucleic acid sequence having at least 80%, 85%, 90%, 91%, 92%, 93%, 94%, 95%, 96%, 97%, 98%, 99% identify to SEQ ID NO: 24.

In some more specific embodiments, provided herein is a vector comprising a nucleic acid sequence of SEQ ID NO: 25, or a nucleic acid sequence having at least 80%, 85%, 90%, 91%, 92%, 93%, 94%, 95%, 96%, 97%, 98%, 99% identify to SEQ ID NO: 25.

5.3.3. Recombinant AAV Particles

In another aspect, provided herein are recombinant AAVs (rAAVs) or rAAV particles comprising a nucleic acid provided herein, and at least an AAV capsid protein. The nucleic acid includes any nucleic acids and rAAV vectors described in Section 5.2 and 5.3.2 above.

The capsid protein may be derived from the same serotype as the ITRs, or a derivative thereof. The capsid may also be of a different serotype than the ITR. For example, in certain embodiments, an AAV particle comprises AAV2 ITRs and an AAV6 capsid (AAV 2/6), AAV2 ITRs and an AAV7 capsid (AAV 2/7), AAV2 ITRs and an AAV8 capsid (AAV 2/8), or AAV2 ITRs and an AAV9 capsid (AAV 2/9).

Naturally occurring AAV capsids comprise AAV VP1, VP2 and VP3 capsid proteins, which are each encoded by splice variants of the AAV cap gene. In general, an AAV particle comprises three proteins, VP1, VP2 and VP3, with VP2 and VP3 being truncated version of VP1 so having sequences that are also comprised by VP1. Generally, the amino acid sequence of VP1 defines the serotype of the capsid. Thus, for example, if the VP1 capsid protein encodes for an AAV2 VP1 protein, AAV will be of the AAV2 serotype, whereas if the VP1 capsid protein encodes an AAV8 VP1 protein, the AAV will be of the AAV8 serotype.

In some embodiments, an AAV capsid protein (e.g., VP1, VP2 and/or VP3) in the present rAAV particle is not a naturally occurring capsid protein. In some embodiments, an AAV capsid protein (e.g., VP1, VP2 and/or VP3) is derived from a naturally occurring capsid protein.

In some embodiments, the AAV capsid protein is a VP1 capsid protein. In other embodiments, the AAV capsid protein is a VP2 capsid protein. In other embodiments, the AAV capsid protein is a VP3 capsid protein. In some embodiments, the rAAV particle comprises a VP1 capsid protein, a VP2 capsid protein and/or a VP3 capsid protein. In other embodiments, the rAAV particle comprises a VP1 capsid protein, a VP2 capsid protein and a VP3 capsid protein. In some embodiments, the rAAV particle comprises a VP1 capsid protein, a VP2 capsid protein and/or a VP3 capsid protein, wherein the capsid proteins of the rAAV particle are of the same serotype. In other embodiments, the rAAV particle comprises a VP1 capsid protein, a VP2 capsid protein and a VP3 capsid protein, wherein the capsid proteins of the AAV particle are of the same serotype.

In certain aspects, the capsid protein is a variant capsid protein. A variant capsid protein may comprise one or more mutations, e.g. amino acid substitutions, amino acid deletions, and heterologous peptide insertions, compared to a corresponding reference capsid protein such as the naturally occurring parental capsid protein, i.e. the capsid protein from which it was derived. In some embodiments the amino acid sequence of the AAV capsid protein is identical to the amino acid sequence of the wild type, or reference, or parent AAV capsid protein except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues, e.g., except for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residue substitutions. In some embodiments, the capsid protein or AAV particle described herein may be a chimeric capsid protein or AAV particle, respectively, comprising a protein sequence of two or more AAV serotype capsid proteins or particles, respectively.

In some embodiments, the capsid protein in the rAAV particle provided herein is derived from an AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9 capsid protein. In specific embodiments, the capsid protein in the rAAV particle provided herein has an amino acid sequence that is at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% identical to the amino acid sequence of an AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9 capsid protein.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV1.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV2.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV2i8.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV3.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV3-B.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV4.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV5.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV6.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV7.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV8.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh8.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh8R.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV9.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV10.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh10.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV11.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV12.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV13.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV-DJ.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV LK03.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAVrh74.

In certain embodiments, an AAV particle provided herein comprises VP1, VP2 and/or VP3 capsid proteins that comprises a VP1, VP2 and/or VP3 capsid protein sequence having at least 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 100% sequence identity, to any of the VP1, VP2 or VP3 amino acid sequences of AAV44-9.

In some specific embodiments, the rAAV particle provided herein comprises a nucleic acid encoding the protein of interest provided herein and VP1 of an AAV comprising an amino acid sequence of SEQ ID NO: 26, SEQ ID NO: 27, SEQ ID NO: 28, SEQ ID NO: 29, SEQ ID NO: 30, SEQ ID NO: 31 or SEQ ID NO: 32. In a specific embodiment, the VP1 comprises an amino acid sequence of SEQ ID NO: 29.

TABLE 4
Exemplary VP1, VP2 and VP3 Proteins

| AAV | Sequence | SEQ ID NO |
|---|---|---|
| AAV2 (UniProt: | MAADGYLPDWLEDTLSEGIRQWWKLKPGPPP | SEQ ID NO: 26 |
| P03135-1) | PKPAERHKDDSRGLVLPGYKYLGPFNGLDKG | |
| VP1: amino acids | EPVNEADAAALEHDKAYDRQLDSGDNPYLKY | |
| 1-735 | NHADAEFQERLKEDTSFGGNLGRAVFQAKKR | |
| VP2: amino acids | VLEPLGLVEEPVKTAPGKKRPVEHSPVEPDS | |
| 138-735 | SSGTGKAGQQPARKRLNFGQTGDADSVPDPQ | |
| VP3: amino acids | PLGQPPAAPSGLGTNTMATGSGAPMADNNEG | |
| 203-735 | ADGVGNSSGNWHCDSTWMGDRVITTSTRTWA | |
| | LPTYNNHLYKQISSQSGASNDNHYFGYSTPW | |
| | GYFDFNRFHCHFSPRDWQRLINNNWGFRPKR | |
| | LNFKLFNIQVKEVTQNDGTTTIANNLTSTVQ | |
| | VFTDSEYQLPYVLGSAHQGCLPPFPADVFMV | |
| | PQYGYLTLNNGSQAVGRSSFYCLEYFPSQML | |
| | RTGNNFTFSYTFEDVPFHSSYAHSQSLDRLM | |
| | NPLIDQYLYYLSRTNTPSGTTTQSRLQFSQA | |
| | GASDIRDQSRNWLPGPCYRQQRVSKTSADNN | |
| | NSEYSWTGATKYHLNGRDSLVNPGPAMASHK | |
| | DDEEKFFPQSGVLIFGKQGSEKTNVDIEKVM | |
| | ITDEEEIRTTNPVATEQYGSVSTNLQRGNRQ | |
| | AATADVNTQGVLPGMVWQDRDVYLQGPIWAK | |
| | IPHTDGHFHPSPLMGGFGLKHPPPQILIKNT | |
| | PVPANPSTTFSAAKFASFITQYSTGQVSVEI | |
| | EWELQKENSKRWNPEIQYTSNYNKSVNVDFT | |
| | VDTNGVYSEPRPIGTRYLTRNL | |
| AAV8 | MAADGYLPDWLEDNLSEGIREWWALKPGAPK | SEQ ID NO: 27 |
| (Uniprot | PKANQQKQDDGRGLVLPGYKYLGPFNGLDKG | |
| Q8JQF8_9VIRU) | EPVNAADAAALEHDKAYDQQLQAGDNPYLRY | |
| VP1: amino acids | NHADAEFQERLQEDTSFGGNLGRAVFQAKKR | |
| 1-738; | VLEPLGLVEEGAKTAPGKKRPVEPSPQRSPD | |
| VP2: amino acids | SSTGIGKKGQQPARKRLNFGQTGDSESVPDP | |
| 138-738; | QPLGEPPAAPSGVGPNTMAAGGGAPMADNNE | |
| VP3: amino acids | GADGVGSSSGNWHCDSTWLGDRVITTSTRTW | |
| 204-738. | ALPTYNNHLYKQISNGTSGGATNDNTYFGYS | |
| | TPWGYFDFNRFHCHFSPRDWQRLINNNWGFR | |
| | PKRLSFKLFNIQVKEVTQNEGTKTIANNLTS | |
| | TIQVFTDSEYQLPYVLGSAHQGCLPPFPADV | |
| | FMIPQYGYLTLNNGSQAVGRSSFYCLEYFPS | |
| | QMLRTGNNFQFTYTFEDVPFHSSYAHSQSLD | |
| | RLMNPLIDQYLYYLSRTQTTGGTANTQTLGF | |
| | SQGGPNTMANQAKNWLPGPCYRQQRVSTTTG | |
| | QNNNSNFAWTAGTKYHLNGRNSLANPGIAMA | |
| | THKDDEERFFPSNGILIFGKQNAARDNADYS | |
| | DVMLTSEEEIKTTNPVATEEYGIVADNLQQQ | |
| | NTAPQIGTVNSQGALPGMVWQNRDVYLQGPI | |
| | WAKIPHTDGNFHPSPLMGGFGLKHPPPQILI | |
| | KNTPVPADPPTTFNQSKLNSFITQYSTGQVS | |
| | VEIEWELQKENSKRWNPEIQYTSNYYKSTSV | |
| | DFAVNTEGVYSEPRPIGTRYLTRNL | |
| AAVrh8 | MAADGYLPDWLEDNLSEGIREWWDLKPGAPK | SEQ ID NO: 28 |
| (Uniprot | PKANQQKQDDGRGLVLPGYKYLGPFNGLDKG | |
| Q808Y3_9VIRU) | EPVNAADAAALEHDKAYDQQLKAGDNPYLRY | |
| VP1: amino acids | NHADAEFQERLQEDTSFGGNLGRAVFQAKKR | |
| 1-736 | VLEPLGLVEEGAKTAPGKKRPVEQSPQEPDS | |
| VP2: amino acids | SSGIGKTGQQPAKKRLNFGQTGDSESVPDPQ | |
| 138-736 | PLGEPPAAPSGLGPNTMASGGGAPMADNNEG | |
| VP3: amino acids | ADGVGNSSGNWHCDSTWLGDRVITTSTRTWA | |
| 203-736 | LPTYNNHLYKQISNGTSGGSTNDNTYFGYST | |
| | PWGYFDFNRFHCHFSPRDWQRLINNNWGFRP | |
| | KRLNFKLFNIQVKEVTTNEGTKTIANNLTST | |
| | VQVFTDSEYQLPYVLGSAHQGCLPPFPADVF | |
| | MVPQYGYLTLNNGSQALGRSSFYCLEYFPSQ | |
| | MLRTGNNFQFSYTFEDVPFHSSYAHSQSLDR | |
| | LMNPLIDQYLYYLVRTQTTGTGGTQTLAFSQ | |
| | AGPSSMANQARNWVPGPCYRQQRVSTTTNQN | |
| | NNSNFAWTGAAKFKLNGRDSLMNPGVAMASH | |
| | KDDDDRFFPSSGVLIFGKQGAGNDGVDYSQV | |
| | LITDEEEIKATNPVATEEYGAVAINNQAANT | |
| | QAQTGLVHNQGVIPGMVWQNRDVYLQGPIWA | |
| | KIPHTDGNFHPSPLMGGFGLKHPPPQILIKN | |
| | TPVPADPPLTFNQAKLNSFITQYSTGQVSVE | |
| | IEWELQKENSKRWNPEIQYTSNYYKSTNVDF | |
| | AVNTEGVYSEPRPIGTRYLTRNL | |
| AAV9 | MAADGYLPDWLEDNLSEGIREWWALKPGAPQ | SEQ ID NO: 29 |
| (Uniprot | PKANQQHQDNARGLVLPGYKYLGPGNGLDKG | |
| Q6JC40_9VIRU) | EPVNAADAAALEHDKAYDQQLKAGDNPYLKY | |
| VP1: amino acids | NHADAEFQERLKEDTSFGGNLGRAVFQAKKR | |
| 1-736 | LLEPLGLVEEAAKTAPGKKRPVEQSPQEPDS | |
| VP2: amino acids | SAGIGKSGAQPAKKRLNFGQTGDTESVPDPQ | |
| 138-736 | PIGEPPAAPSGVGSLTMASGGGAPVADNNEG | |
| VP3: amino acids | ADGVGSSSGNWHCDSQWLGDRVITTSTRTWA | |
| 203-736 | LPTYNNHLYKQISNSTSGGSSNDNAYFGYST | |
| | PWGYFDFNRFHCHFSPRDWQRLINNNWGFRP | |
| | KRLNFKLFNIQVKEVTDNNGVKTIANNLTST | |
| | VQVFTDSDYQLPYVLGSAHEGCLPPFPADVF | |
| | MIPQYGYLTLNDGSQAVGRSSFYCLEYFPSQ | |
| | MLRTGNNFQFSYEFENVPFHSSYAHSQSLDR | |
| | LMNPLIDQYLYYLSKTINGSGQNQQTLKFSV | |
| | AGPSNMAVQGRNYIPGPSYRQQRVSTTVTQN | |
| | NNSEFAWPGASSWALNGRNSLMNPGPAMASH | |
| | KEGEDRFFPLSGSLIFGKQGTGRDNVDADKV | |
| | MITNEEEIKTTNPVATESYGQVATNHQSAQA | |
| | QAQTGWVQNQGILPGMVWQDRDVYLQGPIWA | |
| | KIPHTDGNFHPSPLMGGFGMKHPPPQILIKN | |
| | TPVPADPPTAFNKDKLNSFITQYSTGQVSVE | |
| | IEWELQKENSKRWNPEIQYTSNYYKSNNVEF | |
| | AVNTEGVYSEPRPIGTRYLTRNL | |
| AAVrh10 | MAADGYLPDWLEDNLSEGIREWWDLKPGAPK | SEQ ID NO: 30 |
| (Uniprot | PKANQQKQDDGRGLVLPGYKYLGPFNGLDKG | |
| Q808W5_9VIRU) | EPVNAADAAALEHDKAYDQQLKAGDNPYLRY | |
| VP1: amino acids | NHADAEFQERLQEDTSFGGNLGRAVFQAKKR | |
| 1-738; | VLEPLGLVEEGAKTAPGKKRPVEPSPQRSPD | |
| VP2: amino acids | SSTGIGKKGQQPAKKRLNFGQTGDSESVPDP | |
| 138-738; | QPIGEPPAGPSGLGSGTMAAGGGAPMADNNE | |
| VP3: amino acids | GADGVGSSSGNWHCDSTWLGDRVITTSTRTW | |
| 204-738. | ALPTYNNHLYKQISNGTSGGSTNDNTYFGYS | |
| | TPWGYFDFNRFHCHFSPRDWQRLINNNWGFR | |
| | PKRLNFKLFNIQVKEVTQNEGTKTIANNLTS | |
| | TIQVFTDSEYQLPYVLGSAHQGCLPPFPADV | |
| | FMIPQYGYLTLNNGSQAVGRSSFYCLEYFPS | |
| | QMLRTGNNFEFSYQFEDVPFHSSYAHSQSLD | |
| | RLMNPLIDQYLYYLSRTQSTGGTAGTQQLLF | |
| | SQAGPNNMSAQAKNWLPGPCYRQQRVSTTLS | |
| | QNNNSNFAWTGATKYHLNGRDSLVNPGVAMA | |
| | THKDDEERFFPSSGVLMFGKQGAGKDNVDYS | |
| | SVMLTSEEEIKTTNPVATEQYGVVADNLQQQ | |
| | NAAPIVGAVNSQGALPGMVWQNRDVYLQGPI | |
| | WAKIPHTDGNFHPSPLMGGFGLKHPPPQILI | |
| | KNTPVPADPPTTFSQAKLASFITQYSTGQVS | |
| | VEIEWELQKENSKRWNPEIQYTSNYYKSTNV | |
| | DFAVNTDGTYSEPRPIGTRYLTRNL | |
| AAV2v | MAADGYLPDWLEDTLSEGIRQWWKLKPGPPP | SEQ ID NO: 31 |
| VP1: amino acids | PKPAERHKDDSRGLVLPGYKYLGPFNGLDKG | |
| 1-735 | EPVNEADAAALEHDKAYDRQLDSGDNPYLKY | |
| VP2: amino acids | NHADAEFQERLKEDTSFGGNLGRAVFQAKKR | |
| 138-735; | VLEPLGLVEEPVKTAPGKKRPVEHSPVEPDS | |
| VP3: amino acids | SSGTGKAGQQPARKRLNFGQTGDADSVPDPQ | |
| 204-735 | PLGQPPAAPSGLGTNTMATGSGAPMADNNEG | |
| | ADGVGNSSGNWHCDSTWMGDRVITTSTRTWA | |
| | LPTYNNHLYKQISSQSGASNDNHYFGYSTPW | |
| | GYFDFNRFHCHFSPRDWQRLINNNWGFRPKR | |
| | LNFKLFNIQVKEVTQNDGTTTIANNLTSTVQ | |
| | VFTDSEYQLPYVLGSAHQGCLPPFPADVFMV | |
| | PQYGYLTLNNGSQAVGRSSFYCLEYFPSQML | |
| | RTGNNFTFSYTFEDVPFHSSYAHSQSLDRLM | |
| | NPLIDQYLYFLSRTNTPSGTTTQSRLQFSQA | |
| | GASDIRDQSRNWLPGPCYRQQGVSKVSADNN | |
| | NSEFSWTGATKYHLNGRDSLVNPGPAMASHK | |
| | DDEEKFFPQSGVLIFGKQGSEKTNVDIEKVM | |
| | ITDEEEIRTTNPVATEQYGSVSTNLQSGNTQ | |
| | AATADVNTQGVLPGMVWQDRDVYLQGPIWAK | |
| | IPHTDGHFHPSPLMGGFGLKHPPPQILIKNT | |
| | PVPANPSTTFSAAKFASFITQYSTGQVSVEI | |
| | EWELQKENSKRWNPEIQYTSNYNKSVNVDFT | |
| | VDTNGVYSEPRPIGTRFLTRNL | |
| AAV44-9 | MAADGYLPDWLEDNLSEGIREWWDLKPGAPK | SEQ ID NO: 32 |
| VP1: amino acids | PKANQQKQDDGRGLVLPGYKYLGPFNGLDKG | |
| 1-736 | EPVNAADAAALEHDKAYDQQLKAGDNPYLRY | |
| VP2: amino acids | NHADAEFQERLQEDTSFGGNLGRAVFQAKKR | |
| 138-736; | VLEPLGLVEEGAKTAPGKKRPVEQSPQEPDS | |
| VP3: amino acids | SSGIGKTGQQPAKKRLNFGQTGDTESVPDPQ | |
| 204-736 | PLGEPPAAPSGLGPNTMASGGGAPMADNNEG | |
| | ADGVGNSSGNWHCDSTWLGDRVITTSTRTWA | |
| | LPTYNNHLYKQISNGTSGGSTNDNTYFGYST | |
| | PWGYFDFNRFHCHFSPRDWQRLINNNWGFRP | |
| | KRLNFKLFNIQVKEVTTNEGTKTIANNLTST | |
| | VQVFTDSEYQLPYVLGSAHQGCLPPFPADVF | |
| | MVPQYGYLTLNNGSQALGRSSFYCLEYFPSQ | |
| | MLRTGNNFQFSYTFEDVPFHSSYAHSQSLDR | |
| | LMNPLIDQYLYYLVRTQTTGTGGTQTLAFSQ | |
| | AGPSNMASQARNWVPGPSYRQQRVSTTTNQN | |
| | NNSNFAWTGAAKFKLNGRDSLMNPGVAMASH | |
| | KDDEDRFFPSSGVLIFGKQGAGNDGVDYSQV | |
| | LITDEEEIKATNPVATEEYGAVAINNQAANT | |
| | QAQTGLVHNQGVIPGMVWQNRDVYLQGPIWA | |
| | KIPHTDGNFHPSPLMGGFGLKHPPPQILIKN | |
| | TPVPADPPLTFNQAKLNSFITQYSTGQVSVE | |
| | IEWELQKENSKRWNPEIQYTSNYYKSTNVDF | |
| | AVNTEGVYSEPRPIGTRYLTRNL | |

The rAAV particles described herein may be produced using any suitable method known in the art. For example, a host cell (e.g., a mammalian cell) may be engineered to stably express the necessary components for AAV particle production. This can be achieved by integrating a plasmid (or multiple plasmids) comprising AAV rep and cap genes, and a selectable marker, such as an antibiotic (e.g., neomycin or ampicillin) resistance gene into the genome of the cell. The cell can be, e.g., an insect or mammalian cell which can then be co-infected with a helper virus (e.g., adenovirus or baculovirus providing the helper functions) and the rAAV vector comprising the 5′ and 3′ AAV ITR. The use of a selectable marker allows for large-scale production of the rAAV. As another non-limiting example, adenovirus or baculovirus rather than plasmids can be used to introduce rep and cap genes into packaging cells. As yet another non-limiting example, both the viral vector containing the 5′ and 3′ AAV ITRs and the rep and cap genes can be stably integrated into the DNA of producer cells, and the helper functions can be provided by a wild-type adenovirus to produce the rAAV.

A helper virus for AAV refers to a virus that allows AAV to be replicated and packaged by a host cell. A helper virus provides helper functions that allow for the replication of AAV. A number of such helper viruses have been identified, including adenoviruses, herpesviruses and poxviruses such as vaccinia. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Examples of adenovirus helper functions for the replication of AAV include E1A functions, E1B functions, E2A functions, VA functions and E4orf6 functions.

A preparation of AAV is said to be substantially free of helper virus if the ratio of infectious AAV particles to infectious helper virus particles is at least about 102:1; at least about 104:1, at least about 106:1; or at least about 108:1. Preparations can also be free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

In certain embodiments, host cells containing the rAAV vectors described above is rendered capable of providing AAV helper functions to replicate and encapsulate the polynucleotide encoding the protein of interest provided herein flanked by the AAV LTRs to produce rAAV particles. AAV helper functions are generally AAV-derived coding sequences which can be expressed to provide AAV gene products that, in turn, function in trans for productive AAV replication. AAV helper functions are used herein to complement necessary AAV functions that are missing from the rAAV vectors. In some embodiments, AAV helper functions include one, or both of the major AAV ORFs, namely the rep and cap coding regions, or functional homologues thereof.

AAV helper functions can be introduced into the host cell by transfecting the host cell with an AAV helper construct either prior to, or concurrently with, the transfection of the rAAV vector. For example, AAV helper constructs can be used to provide at least transient expression of AAV rep and/or cap genes to complement missing AAV functions that are necessary for productive AAV infection. Typically, AAV helper constructs lack AAV ITRs and can neither replicate nor package themselves. The AAV helper constructs can be in the form of, e.g., a plasmid, phage, transposon, cosmid, virus, or virion.

In certain embodiments, the host cell is also capable of providing or is provided with non AAV-derived functions or “accessory functions” to produce rAAV particles. Accessory functions are non AAV-derived viral and/or cellular functions upon which AAV is dependent for its replication, such as non AAV proteins and RNAs that are required in AAV replication, including those involved in activation of AAV gene transcription, stage specific AAV mRNA splicing, AAV DNA replication, synthesis of Cap expression products and AAV capsid assembly. In some embodiments, viral-based accessory functions can be derived from a known helper virus.

In some embodiments, as a result of the infection of the host cell with a helper virus and/or an accessory function vector, a recombinant AAV particle is produced, and the produced rAAV particle is infectious, replication-defective virus, and includes an AAV protein shell that encapsulates a heterologous nucleotide sequence of interest flanked on both sides by AAV

rAAV particles can be purified from the host cell using a purification method known in the art, such as chromatography, CsCl gradients, and other methods as described, for example, in U.S. Pat. Nos. 6,989,264 and 8,137,948 and WO 2010/148143. In some embodiments, residual helper virus can be inactivated using known methods, e.g., by heating.

5.3.4. Cells

A variety of host cells can be used to produce rAAV particles described herein. Suitable host cells for producing AAV particles from the polynucleotides and AAV vectors provided herein include microorganisms, yeast cells, insect cells, and mammalian cells. Typically, such cells can be, or have been, used as recipients of a heterologous nucleic acid molecule and can grow in, e.g., suspension culture and a bioreactor.

In some embodiments, the cell is a mammalian host cell, for example, a HEK293, HEK293-T, A549, WEHI, 10T1/2, BHK, MDCK, COS1, COS7, BSC 1, BSC 40, BMT 10, VERO, W138, HeLa, 293, Jurkat, 2V6.11, Saos, C2C12, L, HT1080, HepG2, primary fibroblast, hepatocyte, and myoblast cells.

In other embodiments, the cell is an insect cell, for example an Sf9, SF21, SF900+, or a Drosophila cell lines, mosquito cell lines, e.g., Aedes albopictus derived cell lines, domestic silkworm cell lines, e.g. Bombyxmori cell lines, Trichoplusia ni cell lines such as High Five cells or Lepidoptera cell lines such as Ascalapha odorata cell lines. In some embodiments, insect cells are cells from the insect species which are susceptible to baculovirus infection, including High Five, Sf9, Se301, SeIZD2109, SeUCR1, SP900+, Sf21, BTI-TN-5B1-4, MG-1, Tn368, HzAm1, BM-N, Ha2302, Hz2E5 and Ao38. For example, large scale production of recombinant AAV in cells, including Sf9 insect cells, has been described by Kotin R M. Hum Mol Genet. 20(R1):R2-R6 (2011) doi:10.1093/hmg/ddr141. Methodology for molecular engineering and expression of polypeptides in insect cells is described, for example, in Summers and Smith. A Manual of Methods for Baculovirus Vectors and Insect Culture Procedures, Texas Agricultural Experimental Station Bull. No. 7555, College Station, Tex. (1986); King, L. A. and R. D. Possee, The baculovirus expression system, Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995).

5.4. Pharmaceutical Compositions

In one aspect, the present disclosure further provides pharmaceutical compositions comprising the vector or viral particle of the present disclosure. In some embodiments, a pharmaceutical composition comprises a therapeutically effective amount of the vectors or viral particles provided herein and a pharmaceutically acceptable excipient.

In some embodiments, provided herein is a pharmaceutical composition comprising a therapeutically effective amount of the rAAV vectors provided herein and a pharmaceutically acceptable excipient.

In other embodiments, provided herein is a pharmaceutical composition comprising a therapeutically effective amount of the rAAV particles provided herein and a pharmaceutically acceptable excipient.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, Pa. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, viral particle, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they are preferably sterile. The pharmaceutical composition may be rendered sterile by filtration through sterile filtration membranes. The pharmaceutical compositions herein generally can be placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, intravitreal, subretinal injection, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The pharmaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the therapeutic molecule provided herein, construction of a nucleic acid as part of a viral vector or other vector, etc.

In some embodiments, the pharmaceutical composition provided herein contains the binding molecules and/or viral particles in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

### 5.5. Methods and Uses

In another aspect, provided herein are methods for using and uses of the vectors, or the viral particles (rAAV) provided herein.

Such methods and uses include therapeutic methods and uses, for example, involving administration of the molecules, rAAV or compositions containing the same, to a subject having a disease or disorder. In some embodiments, the molecule, viral particle, and/or composition is administered in an effective amount to effect treatment of the disease or disorder. Uses include uses of viral particles in such methods and treatments, and in the preparation of a medicament in order to carry out such therapeutic methods. In some embodiments, the methods are carried out by administering the viral particles, or compositions comprising the same, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as SMA). This delay can be of varying lengths of time, depending on the history of the disease and/or individual being treated. As is evident to one skilled in the art, a sufficient or significant delay can, in effect, encompass prevention, in that the individual does not develop the disease or disorder.

In other embodiments, the method or the use provided herein prevents a disease or disorder.

In some embodiments, the disease or disorder is associated with SMN. In some embodiments, the disease or disorder is associated with insufficient expression of SMN protein. In some embodiments, the disease or disorder is associated with a deficient SMN protein (such as a mutant SMN protein). In some embodiments, the vectors or viral particles described herein are used to treat a subject with SMA who has a smn1 deletion and/or mutation. In some embodiments, the subject has one or more smn1 mutations or micro deletions. In some embodiments, the subject has one or more smn1 nonsense mutations. In some embodiments, the subject has one or more smn1 frame shift mutations.

In some specific embodiments, the disease or disorder is SMA. In certain embodiments, the present disclosure provides methods of gene therapy for SMN (e.g., SMN1) associated diseases or disorders, for example neuromuscular degenerative diseases, such as SMA-I, SMA-II, SMA-III, and SMA-IV. In some embodiments, the disease or disorder is a neuromuscular degenerative disease such as SMA type I, type II, III and IV. In some specific embodiments, the disease or disorder is SMA type 1. In some embodiments, the disease or disorder is SMA type II. In other embodiments, the disease or disorder is SMA type III. In yet other embodiments, the disease or disorder is SMA type IV.

A microdeletion and non-sense mutation in the smn1 gene is the most frequent genetic cause of SMA-I. The vast majority of neurologically healthy individuals have enough level of SMN1 protein expression detected in blood. Lack of SMN protein (e.g., SMN1) expression has also been identified as a cause of other neurodegenerative diseases, including for example, Parkinson disease, progressive supranuclear palsy, ataxia, corticobasal syndrome, Huntington disease-like syndrome, Creutzfeldt-Jakob disease and Alzheimer disease. In some embodiment, the SMN associated disease or disorder is a SMN protein expression deficiency associated disease (e.g., SMN1 expression deficiency associated disease).

Spinal muscular atrophy (SMA), an early-onset neuromuscular degenerative disorder, is a progressive and fatal disease characterized by, e.g., the selective death of motor neurons in the motor cortex, brainstem and spinal cord. Patients diagnosed with SMA-I develop a progressive muscle phenotype characterized by, e.g., spasticity, hyperreflexia or hyporeflexia, fasciculations, muscle atrophy and paralysis. These motor impairments are caused by the denervation of muscles due to the loss of motor neurons. The major pathological features of SMA-I include degeneration of the corticospinal tracts and extensive loss of lower motor neurons (LMNs) or anterior horn cells, degeneration and loss of Betz cells and other pyramidal cells in the primary motor cortex and reactive gliosis in the motor cortex and spinal cord. SMA-I is usually fatal within 0.9 to 2 years after the diagnosis due to respiratory defects and/or inflammation.

In some embodiments, the symptoms of SMA include, but are not limited to, motor neuron degeneration, muscle weakness, muscle atrophy, the stiffness of muscle, difficulty in breathing, slurred speech, fasciculation development, frontotemporal dementia and/or premature death are improved in the subject treated. In other aspects, the composition of the present disclosure is applied to one or both of the brain and the spinal cord. According to some embodiments, one or both of muscle coordination and muscle function are improved. According to some embodiments, the survival of the subject is prolonged.

In some embodiments, overall enhancement of expression of the AAV derived wild-type smn1 or codon-optimized smn1 reduces the effects of SMA in a subject.

In some embodiments, administration of the composition of the disclosure to a subject may enhance smn1 (e.g. wild type smn1 or codon optimized smn1) mRNA transcription in the transduced cells of a subject. In some embodiments, the transcription of wild-type smn1 and/or codon-optimized smn1 may be enhanced by about 30%, 40%, 50%, 60%, 70%, 80%, 85%, 90%, 95%, 100%, 200%, 300% or 500%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%, 100-500% in the transduced cells, including a region of the CNS, or a specific cell of the CNS of a subject.

In some embodiments, the vectors or viral particles described herein may be administered to a subject who is in the early stages of SMA. Early stage symptoms include, but are not limited to, muscles which are weak and soft or stiff, tight and spastic, cramping and twitching (fasciculations) of muscles, loss of muscle bulk (atrophy), fatigue, poor balance, slurred words, weak grip, and/or tripping when walking. The symptoms may be limited to a single body region or a mild symptom may affect more than one region. As a non-limiting example, administration of the vectors or particles described herein, may reduce the severity and/or occurrence of the symptoms of early stage SMA.

In other embodiments, the vectors or viral particles described herein may be administered to a subject who is in the middle stages of SMA-I or late stage of SMA-I, or early stage of SMA-II-IV. The middle stage of SMA-I or late stage of SMA-I, or early stage of SMA-II-IV include, but are not limited to, more widespread muscle symptoms as compared to the early stage, some muscles are paralyzed while others are weakened or unaffected, continued muscle twitching (fasciculations), unused muscles may cause contractures where the joints become rigid, painful and sometimes deformed, weakness in swallowing muscles may cause choking and greater difficulty eating and managing saliva, weakness in breathing muscles can cause respiratory insufficiency which can be prominent when lying down, and/or a subject may have bouts of uncontrolled and inappropriate laughing or crying (pseudobulbar affect). As a non-limiting example, administration of the vectors or viral particles described herein may reduce the severity and/or occurrence of the symptoms of middle stage SMA-I or late stage SMA-I, or early stage SMA-II-IV.

The compositions described herein can be administered to an individual by any route, e.g., intravascularly (e.g., intravenously (IV) or intraarterially), directly into arteries, systemically (for example by intravenous injection), or locally (for example by intraarterial or intraocular injection). Non-limiting exemplary administration methods include intravenous (e.g., by infusion pumps), intraperitoneal, intraocular, intra-arterial, intrapulmonary, oral, inhalation, intravesicular, intramuscular, intra-tracheal, subcutaneous, intraocular, intrathecal, transdermal, transpleural, intraarterial, topical, inhalational (e.g., as mists of sprays), mucosal (such as via nasal mucosa), subcutaneous, transdermal, gastrointestinal, intraarticular, intracisternal, intraventricular, intracranial, intraurethral, intrahepatic, intratumoral, intravitreal and subretinal injection. In some embodiments, the composition of the present disclosure for treating SMA is administered to the subject in need thereof intravenously, intramuscularly, subcutaneously, intraperitoneally, intrathecally and/or intraventricularly, allowing the present nucleic acid to pass through one or both the blood-brain barrier and the blood spinal cord barrier. In some embodiments, the method includes administering (e.g., intraventricularly administering and/or intrathecally administering) directly to the central nervous system (CNS) of a subject (using, e.g., an infusion pump and/or a delivery scaffold) a therapeutically effective amount of a composition of the present disclosure. The vector or viral particle may be used to enhance smn1 gene expression, and/or reducing one or more symptoms of SMA in the subject such that SMA is therapeutically treated.

In some embodiments, compositions of AAV vector or AAV particle comprising a nucleic acid sequence described herein may be administered in a way which facilitates the composition to enter the central nervous system and penetrate into motor neurons.

In some embodiments, the AAV vector or AAV particle of the present disclosure may be administered by muscular injection.

In some embodiments, the AAV vector or AAV particle that comprises the nucleic acid of the present disclosure is administered to a subject by peripheral injections and/or intranasal delivery.

In some embodiments, the AAV vector or AAV particle that comprises the nucleic acid of the present disclosure is administered to a subject by intracranial delivery (e.g. intrathecal or intracerebroventricular administration, see e.g., U.S. Pat. No. 8,119,611; the content of which is incorporated herein by reference in its entirety).

In some embodiments, the composition comprising the AAV vector or particle of the present disclosure is administered intravenous or intracranial to the central nervous system (CNS) of the subject. In other embodiments, the composition comprising the AAV vector or particle of the present disclosure is administered to CNS, such as neurons, motor neurons, microglia and astrocytes. In other embodiments, the composition comprising the AAV vector or particle of the present disclosure is administered to astrocytes.

In some embodiments, the composition comprising the AAV vector or particle of the present disclosure may be delivered into specific types of targeted cells, including neurons, motor neurons; glial cells including oligodendrocyte, astrocyte and microglia; and/or other cells surrounding neurons such as T cells.

In some embodiments, the AAV vector or AAV particle of the present disclosure may be administered in a therapeutically effective amount, e.g., an amount that is sufficient to alleviate and/or prevent at least one symptom associated with the disease, or provide improvement in the condition of the subject.

In some embodiments, the AAV vector or AAV particle of the present disclosure may be administered to the CNS in a therapeutically effective amount to improve function and/or survival for a subject with SMA. As a non-limiting example, the composition may be administered intravenously and/or intrathecally.

In some embodiments, the AAV vector or AAV particle of the present disclosure may be administered to a subject (e.g., to the CNS of a subject) in a therapeutically effective amount to slow the functional decline of a subject (e.g., determined using a known evaluation method such as the SMA functional rating scale (SMAFRS)) and/or prolong ventilator-independent survival of subjects (e.g., decreased mortality or need for ventilation support). As a non-limiting example, the composition may be administered intravenously and/or intrathecally.

In some embodiments, the AAV vector or AAV particle of the present disclosure may be administered to the cisterna magna in a therapeutically effective amount to transduce spinal cord motor neurons and/or astrocytes. As a non-limiting example, the composition may be administered intrathecally.

In some embodiments, the AAV vector or AAV particle of the present disclosure may be administered using intrathecal infusion in a therapeutically effective amount to transduce spinal cord motor neurons and/or astrocytes. As a non-limiting example, the composition may be administered intrathecally.

In some embodiments, the AAV vector or AAV particle of the present disclosure may be administered using a bolus infusion.

In some embodiments, the AAV vector or AAV particle of the present disclosure may be administered using sustained delivery over a period of minutes, hours or days. The infusion rate may be changed depending on the subject, distribution, formulation or another delivery parameter.

In some embodiments, the catheter may be located at more than one site in the spine for multi-site delivery. In some embodiments, the AAV vector or AAV particle of the present disclosure may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In some embodiments, a subject may be analyzed for spinal anatomy and pathology prior to delivery of the AAV vector or AAV particle described herein. As a non-limiting example, a subject with scoliosis may have a different dosing regimen and/or catheter location compared to a subject without scoliosis.

In some embodiments, the orientation of the spine of the subject during delivery of the present AAV vector or particle may be vertical to the ground. In other embodiments, the orientation of the spine of the subject during delivery of the AAV vector or AAV particle may be horizontal to the ground.

In some embodiments, the spine of the subject may be at an angle as compared to the ground during the delivery of the present AAV vector or AAV particle. The angle of the spine of the subject as compared to the ground may be at least 10, 20, 30, 40, 50, 60, 70, 80, 90, 100, 110, 120, 130, 140, 150 or 180 degrees.

In some embodiments, the delivery method and duration is chosen to provide broad transduction in the spinal cord. As a non-limiting example, intrathecal delivery is used to provide broad transduction along the rostral-caudal length of the spinal cord. As another non-limiting example, multi-site infusions provide a more uniform transduction along the rostral-caudal length of the spinal cord. As yet another non-limiting example, prolonged infusions provide a more uniform transduction along the rostral-caudal length of the spinal cord.

The pharmaceutical compositions of the present disclosure may be administered to a subject using any amount effective for reducing, preventing and/or treating a SMN associated disorder (e.g., SMA). The exact amount required may vary from subject to subject, depending on the species, age, and general condition of the subject, the severity of the disease, the particular composition, its mode of administration, its mode of activity, and the like.

In some embodiments, the AAV vector or AAV particle of the present disclosure may be administered in any suitable form, either as a liquid solution or suspension, as a solid form suitable for liquid solution or suspension in a liquid solution, and may be formulated with any appropriate and pharmaceutically acceptable excipient. In some embodiments, the AAV vector or particle is formulated. As a non-limiting example, the basicity and/or osmolality of the formulation may be optimized to ensure optimal drug distribution in the central nervous system or a region or component of the central nervous system.

In some embodiments, the pharmaceutical composition provided herein is a suspension, e.g., a refrigerated suspension. In some embodiments, the method further comprises agitating the suspension to ensure even distribution of the suspension prior to the administration step. In some embodiments, the method further comprises warming the pharmaceutical composition to room temperature prior to the administration step. The compositions may also be administered in a sustained release formulation. The sustained release devices (such as pellets, nanoparticles, microparticles, nanospheres, microspheres, and the like) may be administered by injection or surgical implanted in various locations.

The compositions of the present disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutic effectiveness for any particular patient will depend upon a variety of factors including the disorder being treated and the severity of the disorder; the activity of the specific compound employed; the specific composition employed; the age, body weight, general health, sex and diet of the patient; the time of administration; route of administration; the duration of the treatment; drugs used in combination or coincidental with the specific compound employed; and like factors well known in the medical arts.

In some embodiments, the age and sex of a subject may be used to determine the dose of the compositions of the present disclosure. As a non-limiting example, a subject who is older may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a younger subject. As another non-limiting example, a subject who is younger may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to an older subject. As yet another non-limiting example, a subject who is female may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a male subject. As yet another non-limiting example, a subject who is male may receive a larger dose (e.g., 5-10%, 10-20%, 15-30%, 20-50%, 25-50% or at least 1%, 2%, 3%, 4%, 5%, 10%, 20%, 30%, 40%, 50%, 60%, 70%, 80%, 90% or more than 90% more) of the composition as compared to a female subject.

In some embodiments, the doses of AAV vectors or AAV particles for delivering the nucleic acid of the present disclosure may be adapted dependent on the disease condition, the subject and the treatment strategy.

In some embodiments, the concentration of vector or viral particle that is administered may differ depending on production method and may be chosen or optimized based on concentrations determined to be therapeutically effective for the particular route of administration.

According to some embodiments, the concentration in vector genomes per milliliter (vg/ml) is selected from the group consisting of about 10⁸ vg/ml, about 10⁹ vg/ml, about 10¹⁰ vg/ml, about 10¹¹ vg/ml, about 10¹² vg/ml, about 10¹³ vg/ml, about 10¹⁴ vg/ml, and about 10¹⁵ vg/ml. In some embodiments, the concentration is in the range of 10¹⁰ vg/ml-10¹⁴ vg/ml, for example 10¹⁰ vg/ml-10¹⁵ vg/ml, 10¹⁰ vg/ml-10¹⁴ vg/ml, 010 vg/ml-10¹³ vg/ml, 10¹⁰ vg/ml-10¹² vg/ml, 10¹⁰ vg/ml-10¹¹ vg/ml, 10¹¹ vg/ml-10¹⁴ vg/ml, 10¹¹ vg/ml-10¹³ vg/ml, 10¹¹ vg/ml-10¹² vg/ml, 10¹² vg/ml-10¹⁴ vg/ml, 10¹² vg/ml-10¹³ vg/ml, 10¹³ vg/ml-10¹⁴ vg/ml, or 10¹⁴ vg/ml-10¹⁵ vg/ml. In some embodiments, the vector or viral particle provided herein is delivered by intravenous, intracranial injection, or intra cisterna magna injection, or intrathecal injection, or intramuscular injection, or intravitreal injection. In some embodiments, the vector or viral particle provided herein is injected in a volume between about 0.1 ml and about 20 ml, for example between about 0.1 ml and about 20 ml, between about 0.5 ml and about 20 ml, between about 1 ml and about 20 ml, between about 5 ml and about 20 ml, between about 0.1 ml and about 5.0 ml, between about 0.1 ml and about 2.0 ml, between about 0.1 ml and about 1.0 ml, between about 0.1 ml and about 0.8 ml, between about 0.1 ml and about 0.6 ml, between about 0.1 ml and about 0.4 ml, between about 0.1 ml and about 0.2 ml, between about 0.2 ml and about 1.0 ml, between about 0.2 ml and about 0.8 ml, between about 0.2 ml and about 0.6 ml, between about 0.2 ml and about 0.4 ml, between about 0.4 ml and about 1.0 ml, between about 0.4 ml and about 0.8 ml, between about 0.4 ml and about 0.6 ml, between about 0.6 ml and about 1.0 ml, between about 0.6 ml and about 0.8 ml, between about 0.8 ml and about 1.0 ml, or about 0.1 ml, about 0.2 ml, about 0.4 ml, about 0.6 ml, about 0.8 ml, and about 1.0 ml.

In some embodiments, a rAAV comprising the nucleic acid described herein can be administered to a subject at a dose of 1×10⁸ to 1×10¹⁷ vector genomes (vg), such as 1×10⁹ to 1×10¹⁷ vector genomes (vg) or 1×10¹⁴ to 1×10¹⁵ vector genomes (vg), and including, e.g., 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, 1×10¹⁷, 2×10¹⁷, 3×10¹⁷, 4×10¹⁷, 5×10¹⁷, 6×10¹⁷, 7×10¹⁷, 8×10¹⁷, or 9×10¹⁷ vector genomes (vg).

In some embodiments, a rAAV comprising the nucleic acid described herein can be administered to a subject at a dose of 1×10⁸ to 1×10¹⁷ vector genomes/kg (vg/kg), such as 1×10¹³ to 1×10¹⁶ vg/kg, and including, e.g., 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ vg/kg.

In some embodiments, one or more additional therapeutic agents may be administered to the subject.

The effectiveness of the compositions described herein can be monitored by several criteria. For example, after treatment in a subject using methods of the present disclosure, the subject may be assessed for e.g., an improvement and/or stabilization and/or delay in the progression of one or more signs or symptoms of the disease state by one or more clinical parameters including those described herein. Examples of such tests are known in the art, and include objective as well as subjective (e.g., subject reported) measures.

In some embodiments, the AAV vector or AAV particle of the present disclosure may be delivered to a subject via a single route administration. In other embodiments, the AAV vector or AAV particle of the present disclosure may be delivered to a subject via a multi-site route of administration, e.g., at 2, 3, 4, 5 or more than 5 sites.

Pharmaceutical compositions comprising the present AAV vector or AAV particle may be administered in a single daily dose, or the daily dose may be administered in divided dosages of two, three, or four times daily. Compositions provided herein can also be administered multiple times (e.g., twice, three times, four times, or five times) within a time period (e.g., 1 week, 2 weeks, 3 weeks, 1 month, 2 months, 3 months, 4 months, 5 months, 6 months, 7 months, 8 months, 9 months, 10 months, 11 months, 1 year, 2 years, or 3 years).

In some embodiments, the composition comprising the AAV vector or particle of the present disclosure is administered as solo therapeutics or combination therapeutics for the treatment of SMA.

In some embodiments, the composition comprising the AAV vector or particle of the present disclosure may be used in combination with one or more other therapeutic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent.

In some embodiments, therapeutic agents that may be used in combination with the AAV vector or particle of the present disclosure can be small molecule compounds including, e.g., immunosuppressants, antioxidants, anti-inflammatory agents, anti-apoptosis agents, calcium regulators, antiglutamatergic agents, structural protein inhibitors, and compounds involved in metal ion regulation.

In some embodiments, compounds for treating SMA which may be used in combination with the vectors or viral particles described herein include, but are not limited to, antiglutamatergic agents such as Riluzole, Topiramate, Talampanel, Lamotrigine, Dextromethorphan, Gabapentin and AMPA antagonist; anti-apoptosis agents such as Minocycline, sodium phenylbutyrate and Arimoclomol; anti-inflammatory agent such as ganglioside, Celecoxib, Cyclosporine, Azathioprine, Cyclophosphamide, Plasmaphoresis, Glatiramer acetate and thalidomide; Ceftriaxone; Beat-lactam antibiotics; Pramipexole (a dopamine agonist); Nimesulide; Diazoxide; pyrazolone derivatives; free radical scavengers that inhibit oxidative stress-induced cell death, such as bromocriptine; phenyl carbamate compounds; neuroprotective compounds; and glycopeptides.

In some embodiments, therapeutic agents that may be used in combination therapy with the vectors or viral particles described herein may be hormones or variants that can protect neuronal loss, such as adrenocorticotropic hormone (ACTH) or fragments thereof (e.g., U.S. Patent Publication No. 20130259875); Estrogen (e.g., U.S. Pat. Nos. 6,334,998 and 6,592,845); the content of each of which is incorporated herein by reference in their entirety.

In some embodiments, neurotrophic factors may be used in combination therapy with the AAV vector or AAV particle of the present disclosure for treating SMA. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

### 5.6. Assays

5.6.1. Analysis of mRNA Levels

Enhancement of levels or expression of a gene (e.g., a smn1 nucleic acid) can be assayed in a variety of ways known in the art.

For example, several methods of detecting or quantitating mRNA levels are known in the art. Exemplary methods include, but are not limited to, northern blots, ribonuclease protection assays, PCR-based methods, and the like. The mRNA sequence of a gene can be used to prepare a probe that is at least partially complementary to the mRNA sequence. The probe can then be used to detect the mRNA in a sample, using any suitable assay, such as PCR based methods, northern blotting, a dipstick assay, and the like.

The assay method can be varied depending on the type of mRNA information desired. Exemplary methods include but are not limited to northern blots and PCR-based methods (e.g., qRT-PCR). Methods such as qRT-PCR can also accurately quantitate the amount of the mRNA in a sample.

Any suitable assay platform can be used to determine the presence of mRNA in a sample. For example, an assay may be in the form of a dipstick, a membrane, a chip, a disk, a test strip, a filter, a microsphere, a slide, a multi-well plate, or an optical fiber. An assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film, a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.). See, e.g., Ausubel et al., Short Protocols in Molecular Biology (Wiley & Sons, 3rd ed. 1995); Sambrook et al., Molecular Cloning: A Laboratory Manual (Cold Spring Harbor, N.Y., 3rd ed. 2001). In some embodiments, the sample is labeled with fluorescent label. Exemplary fluorescent dyes include, but are not limited to, xanthene dyes, fluorescein dyes (e.g., fluorescein isothiocyanate (FITC), 6-carboxyfluorescein (FAM), 6 carboxy-2′,4′,7′,4,7-hexachlorofluorescein (HEX), 6-carboxy-4′,5′-dichloro-2′,7′-dimethoxyfluorescein (JOE)), rhodamine dyes (e.g., rhodamine 110 (R110), N,N,N′,N′-tetramethyl-6-carboxyrhodamine (TAMRA), 6-carboxy-X-rhodamine (ROX), 5 carboxyrhodamine 6G (R6G5 or G5), 6-carboxyrhodamine 6G (R6G6 or G6)), cyanine dyes (e.g., Cy3, Cy5 and Cy7), Alexa dyes (e.g., Alexa-fluor-555), coumarin, Diethylaminocoumarin, umbelliferone, benzimide dyes (e.g., Hoechst 33258), phenanthridine dyes (e.g., Texas Red), ethidium dyes, acridine dyes, carbazole dyes, phenoxazine dyes, porphyrin dyes, polymethine dyes, BODIPY dyes, quinoline dyes, Pyrene, Fluorescein Chlorotriazinyl, eosin dyes, Tetramethylrhodamine, Lissamine, Napthofluorescein, and the like.

A typical mRNA assay method can contain the steps of 1) obtaining surface-bound subject probes; 2) hybridizing a population of mRNAs to the surface-bound probes under conditions sufficient to provide for specific binding; (3) post-hybridization washing to remove nucleic acids not specifically bound to the surface-bound probes; and (4) detecting the hybridized mRNAs. The reagents used in each of these steps and their conditions for use may vary depending on the particular application.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions may be employed.

Hybridization is typically performed under stringent hybridization conditions. Standard hybridization techniques (e.g., under conditions sufficient to provide for specific binding of target mRNAs in the sample to the probes) are described in Kallioniemi et al., Science, 258:818-821 (1992) and International Patent Application Publication No. WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al., Meth. Enzymol. 1981, 21:470-480; Angerer et al., Genetic Engineering: Principles and Methods, Vol 7, pgs 43-65 (Plenum Press, New York, Setlow and Hollaender, eds. 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art.

Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

Other methods, such as PCR-based methods, can also be used to detect the expression of a gene. Examples of PCR methods can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, quantitative Reverse Transcription-PCR (qRT-PCR) can be used for both the detection and quantification of RNA targets (Bustin et al., Clin. Sci. 2005, 109:365-379). Quantitative results obtained by qRT-PCR are generally more informative than qualitative data. Thus, in some embodiments, qRT-PCR-based assays can be useful to measure mRNA levels during cell-based assays. The qRT-PCR method is also useful to monitor patient therapy. Examples of qRT-PCR-based methods can be found, for example, in U.S. Pat. No. 7,101,663, which is incorporated by reference herein in its entirety.

In contrast to regular reverse transcriptase-PCR and analysis by agarose gels, qRT PCR gives quantitative results. An additional advantage of qRT-PCR is the relative ease and convenience of use. Instruments for qRT-PCR, such as the Applied Biosystems 7500, are available commercially, so are the reagents, such as TaqMan® Sequence Detection Chemistry. For example, TaqMan® Gene Expression Assays can be used, following the manufacturer's instructions. These kits are pre-formulated gene expression assays for rapid, reliable detection and quantification of human, mouse, and rat mRNA transcripts. To determine the cycle number at which the fluorescence signal associated with a particular amplicon accumulation crosses the threshold (referred to as the CT), the data can be analyzed, for example, using 7500 Real-Time PCR System Sequence Detection software vs. using the comparative CT relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

In other embodiments, a target RNA can be detected or quantified by Next Generation Sequencing (NGS).

5.6.2 Analysis of Protein Levels

Enhanced protein expression of smn1 nucleic acids can be assessed by measuring SMN protein levels. Protein levels can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS), LC-MS (Liquid-chromatography—MassSpec), and other methods. Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of mouse, rat, monkey, and human smn1 are commercially available. In case of MassSpec, protein levels can be measure using labeled or unlabeled methods.

5.6.3. In Vivo Analysis

In vivo assays can be used to assess enhanced expression of smn1, such as, improved motor function and respiration, meanwhile reducing off-target toxicity in liver and/or heart.

In some embodiments, motor function is measured by righting, open field performance in the animal. In certain embodiments, respiration is measured by whole body plethysmograph, invasive resistance, and compliance measurements in the animal.

In some embodiments, overall survival (OS) and disease free survival (DFS) is measured by body weight, health status observation twice daily in the alive animal.

Testing may be performed in normal animals, or in experimental disease models. For administration to animals, oligonucleotides can be formulated in a pharmaceutically acceptable diluent, such as phosphate-buffered saline. Administration includes parenteral routes of administration, such as intraperitoneal, intravenous, and subcutaneous. Calculation of oligonucleotide dosage and dosing frequency is within the abilities of those skilled in the art, and depends upon factors such as route of administration and animal body weight. Following a period of treatment with oligonucleotides, RNA can be isolated from tissues of interest, including liver, heart, spleen, CNS tissue or CSF and changes in smn1 nucleic acid expression are measured, e.g., using NGS.

5.7. Kits and Articles of Manufacture

Further provided are kits, unit dosages, and articles of manufacture comprising any of the compositions described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as SMA) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 6. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 6.1. Example 1—Construction of Nucleic Acids Encoding SMN

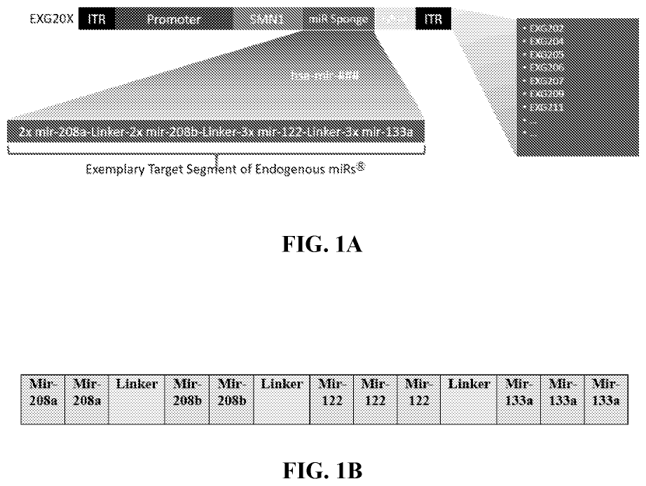

This example illustrates the construction of the exemplary nucleic acids provided herein comprising a first nucleic acid region encoding a SMN and a second nucleic acid region comprising multiple target segments of multiple endogenous microRNAs (miRNAs) (see FIG. 1A).

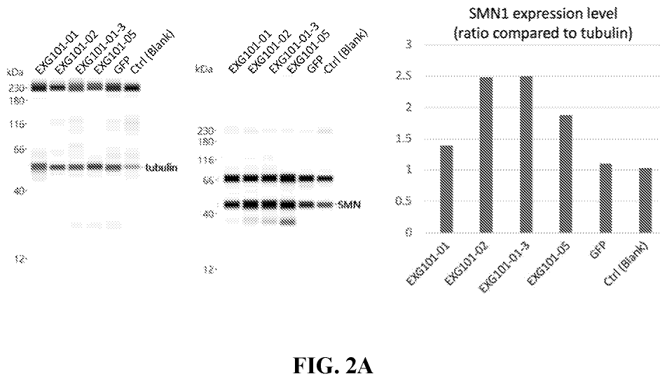

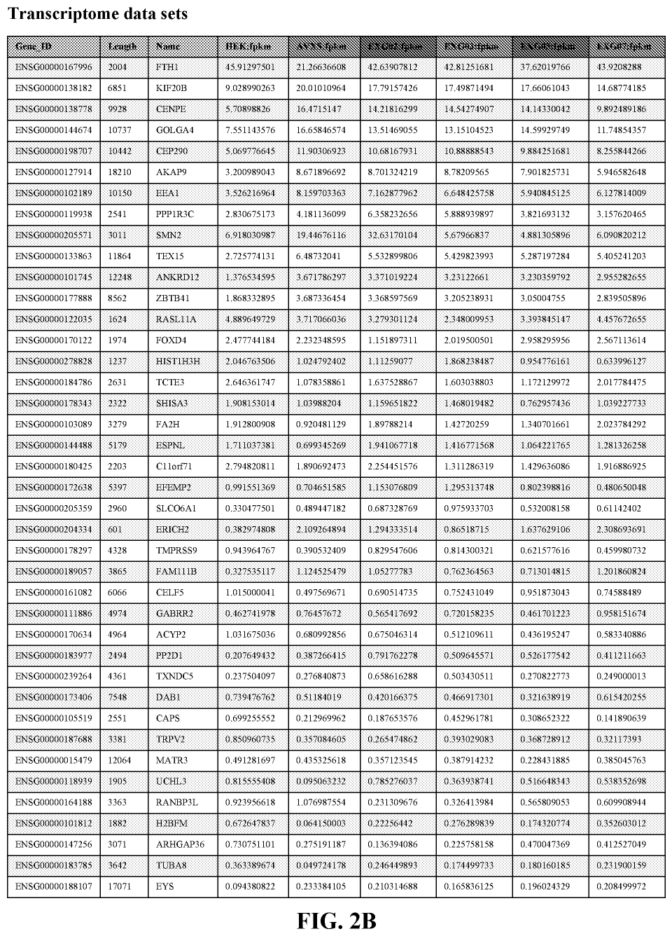

Optimization of the coding sequences of human SMN1 (hSMN1) was performed based on the wild-type SMN1 protein sequence (SEQ ID NO: 33) using algorithms and platforms, including GPS Technology (ATUM Inc., USA), OptimWiz (GeneWiz, USA), OptimumGene (GenScript, USA), and PyCUB (J. Kalfon, 2018). Wild type hSMN1 coding sequence (SEQ ID NO: 34) and codon optimized hSMN1 coding sequences (e.g., SEQ ID NO: 35) were subsequently cloned into expression vectors containing optimized promoter sequences (SEQ ID NO: 36 or SEQ ID NO: 37) provided herein. Codon optimization increased hSMN1 mRNA expression in vitro as measured by transcriptome analysis (FIG. 2B), consistent with protein expression analysis using Western Blot (FIG. 2A) described in more detail below. In addition, as discussed in more detail below, increased median survival of the injected animals was observed as shown in in vivo potency assays.

The mRNA transcript of each target segment used in the present nucleic acid constructs is specifically hybridizable by a tissue-specific endogenous miRNA, for example, liver specific miRNAs such as hsa-mir-122, and heart-specific endogenous miRNAs such as hsa-mir-1-5p, hsa-mir-208a, hsa-mir-208b, hsa-mir-133a, and hsa-mir-448-5p. Exemplary target segment for each of the above-mentioned miRNA is shown in Table 2 above. FIG. 1B illustrates a specific nucleic acid region comprising multiple target segments. The sequences of exemplary second nucleic acid region comprising multiple target segments of multiple endogenous miRNAs are also shown in Table 6 below with the component target segments for each nucleic acid sequences are indicated. In FIG. 1A, FIG. 1B, and Table 6, miR-1 represents a target segment of hsa-mir-1-5p, miR-208a represents a target segment of hsa-mir-208a-5p, miR-208b represents a target segment of hsa-mir-208b-5p, miR-122 represents a target segment of hsa-mir-122, miR-133 represents a target segment of hsa-mir-133a-1, and miR-488 represents a target segment of hsa-mir-488-5p. The 5′ (left) of these exemplary constructs is congruous to SMN coding sequence and its buffering sequence, and the 3′ (right) of these exemplary constructs is congruous to polyA sequence and its buffering sequence. A linker is present between certain target segments. As shown, the constructs can have multiple repeat sequences. The sequences are provided as exemplary constructs, the order of these target segments can be altered and are included in the present disclosure.

TABLE 5
Sequences for SMN and Promoter

| Regions | Sequences | SEQ ID NOS |
|---|---|---|
| SMN protein (wt) | MAMSSGGSGGGVPEQEDSVLFRRGTGQSDDSDI | SEQ ID NO: 33 |
| amino acid | WDDTALIKAYDKAVASFKHALKNGDICETSGKP | |
| sequence | KTTPKRKPAKKNKSQKKNTAASLQQWKVGDKCS | |
| | AIWSEDGCIYPATIASIDFKRETCVVVYTGYGN | |
| | REEQNLSDLLSPICEVANNIEQNAQENENESQV | |
| | STDESENSRSPGNKSDNIKPKSAPWNSFLPPPP | |
| | PMPGPRLGPGKPGLKFNGPPPPPPPPPPHLLSC | |
| | WLPPFPSGPPIIPPPPPICPDSLDDADALGSML | |
| | ISWYMSGYHTGYYMGFRQNQKEGRCSHSLN | |
| SMN nucleic acid | ATGGCGATGAGCAGCGGCGGCAGTGGTGGCGGC | SEQ ID NO: 34 |
| sequence (wt) | GTCCCGGAGCAGGAGGATTCCGTGCTGTTCCGG | |
| | CGCGGCACAGGCCAGAGCGATGATTCTGACATT | |
| | TGGGATGATACAGCACTGATAAAAGCATATGAT | |
| | AAAGCTGTGGCTTCATTTAAGCATGCTCTAAAG | |
| | AATGGTGACATTTGTGAAACTTCGGGTAAACCA | |
| | AAAACCACACCTAAAAGAAAACCTGCTAAGAAG | |
| | AATAAAAGCCAAAAGAAGAATACTGCAGCTTCC | |
| | TTACAACAGTGGAAAGTTGGGGACAAATGTTCT | |
| | GCCATTTGGTCAGAAGACGGTTGCATTTACCCA | |
| | GCTACCATTGCTTCAATTGATTTTAAGAGAGAA | |
| | ACCTGTGTTGTGGTTTACACTGGATATGGAAAT | |
| | AGAGAGGAGCAAAATCTGTCCGATCTACTTTCC | |
| | CCAATCTGTGAAGTAGCTAATAATATAGAACAG | |
| | AATGCTCAAGAGAATGAAAATGAAAGCCAAGTT | |
| | TCAACAGATGAAAGTGAGAACTCCAGGTCTCCT | |
| | GGAAATAAATCAGATAACATCAAGCCCAAATCT | |
| | GCTCCATGGAACTCTTTTCTCCCTCCACCACCC | |
| | CCCATGCCAGGGCCAAGACTGGGACCAGGAAAG | |
| | CCAGGTCTAAAATTCAATGGCCCACCACCGCCA | |
| | CCGCCACCACCACCACCCCACTTACTATCATGC | |
| | TGGCTGCCTCCATTTCCTTCTGGACCACCAATA | |
| | ATTCCCCCACCACCTCCCATATGTCCAGATTCT | |
| | CTTGATGATGCTGATGCTTTGGGAAGTATGTTA | |
| | ATTTCATGGTACATGAGTGGCTATCATACTGGC | |
| | TATTATATGGGTTTTAGACAAAATCAAAAAGAA | |
| | GGAAGGTGCTCACATTCCTTAAAT | |
| Codon optimized | ATGGCCATGAGCAGCGGAGGAAGCGGAGGAGGA | SEQ ID NO: 35 |
| SMN nucleic acid | GTGCCCGAGCAAGAGGACAGCGTGCTGTTTAGG | |
| sequence | AGAGGAACCGGACAGAGCGATGACTCCGATATC | |
| | TGGGACGACACCGCTCTGATCAAGGCCTATGAC | |
| | AAAGCCGTGGCCTCCTTCAAGCACGCTCTGAAG | |
| | AATGGCGATATCTGTGAGACCTCCGGCAAACCT | |
| | AAGACCACCCCCAAGAGGAAGCCCGCCAAGAAG | |
| | AACAAGTCCCAGAAGAAGAATACCGCCGCTAGC | |
| | CTCCAGCAGTGGAAAGTGGGCGATAAGTGCAGC | |
| | GCCATTTGGAGCGAGGATGGATGCATCTACCCC | |
| | GCCACCATTGCCAGCATCGACTTCAAGAGGGAG | |
| | ACATGCGTGGTGGTGTATACCGGATACGGAAAT | |
| | AGAGAGGAGCAGAATCTGAGCGATCTGCTGTCC | |
| | CCCATCTGCGAGGTGGCCAATAATATCGAGCAG | |
| | AACGCCCAAGAGAACGAGAACGAAAGCCAAGTG | |
| | TCCACCGATGAGAGCGAGAACTCCAGAAGCCCC | |
| | GGAAACAAGTCCGACAACATCAAACCCAAGAGC | |
| | GCCCCTTGGAACAGCTTTCTGCCTCCTCCCCCC | |
| | CCCATGCCCGGCCCTAGACTGGGACCCGGCAAG | |
| | CCCGGACTGAAGTTCAACGGACCCCCCCCTCCT | |
| | CCTCCCCCCCCTCCTCCTCATCTGCTGAGCTGC | |
| | TGGCTCCCCCCTTTCCCTAGCGGCCCCCCCATT | |
| | ATCCCCCCCCCTCCCCCTATCTGTCCCGACAGC | |
| | CTCGATGACGCTGACGCCCTCGGAAGCATGCTG | |
| | ATCAGCTGGTACATGAGCGGCTACCACACCGGA | |
| | TACTACATGGGCTTCAGACAGAACCAGAAGGAG | |
| | GGCAGATGCTCCCACTCTCTGAAC | |
| Exemplary | CGTTACATAACTTACGGTAAATGGCCCGCCTGG | SEQ ID NO: 36 |
| Promoter | CTGACCGCCCAACGACCCCCGCCCATTGACGTC | |
| Sequence (1) | AATAATGACGTATGTTCCCATAGTAACGCCAAT | |
| | AGGGACTTTCCATTGACGTCAATGGGTGGAGTA | |
| | TTTACGGTAAACTGCCCACTTGGCAGTACATCA | |
| | AGTGTATCATATGCCAAGTACGCCCCCTATTGA | |
| | CGTCAATGACGGTAAATGGCCCGCCTGGCATTA | |
| | TGCCCAGTACATGACCTTATGGGACTTTCCTAC | |
| | TTGGCAGTACATCTACTCGAGGCCACGTTCTGC | |
| | TTCACTCTCCCCATCTCCCCCCCCTCCCCACCC | |
| | CCAATTTTGTATTTATTTATTTTTTAATTATTT | |
| | TGTGCAGCGATGGGGGCGGGGGGGGGGGGGGGG | |
| | CGCGCGCCAGGCGGGGCGGGGCGGGGCGAGGGG | |
| | CGGGGCGGGGCGAGGCGGAGAGGTGCGGCGGCA | |
| | GCCAATCAGAGCGGCGCGCTCCGAAAGTTTCCT | |
| | TTTATGGCGAGGCGGCGGCGGCGGCGGCCCTAT | |
| | AAAAAGCGAAGCGCGCGGCGGGCGGGAGCGGGA | |
| | TCAGCCACCGCGGTGGCGGCCTAGAGTCGACGA | |
| | GGAACTGAAAAACCAGAAAGTTAACTGGTAAGT | |
| | TTAGTCTTTTTGTCTTTTATTTCAGGTCCCGGA | |
| | TCCGGTGGTGGTGCAAATCAAAGAACTGCTCCT | |
| | CAGTGGATGTTGCCTTTACTTCTAGGCCTGTAC | |
| | GGAAGTGTTACTTCTGCTCTAAAAGCTGCGGAA | |
| | TTGTACCCGCGGCCGATCCACCGGTCCGGAATT | |
| | CCCGGGATATCGTCGACCCACGCGTCCGGGCCC | |
| | CACGCTGCGCACCCGCGGGTTTGCT | |
| Exemplary | CCGTTACATAACTTACGGTAAATGGCCCGCCTG | SEQ ID NO: 37 |
| Promoter | GCTGACCGCCCAACGACCCCCGCCCATTGACGT | |
| Sequence (2) | CAATAGTAACGCCAATAGGGACTTTCCATTGAC | |
| | GTCAATGGGTGGAGTATTTACGGTAAACTGCCC | |
| | ACTTGGCAGTACATCAAGTGTATCATATGCCAA | |
| | GTACGCCCCCTATTGACGTCAATGACGGTAAAT | |
| | GGCCCGCCTGGCATTGTGCCCAGTACATGACCT | |
| | TATGGGACTTTCCTACTTGGCAGTACATCTACG | |
| | TATTAGTCATCGCTATTACCATGGTCGAGGTGA | |
| | GCCCCACGTTCTGCTTCACTCTCCCCATCTCCC | |
| | CCCCCTCCCCACCCCCAATTTTGTATTTATTTA | |
| | TTTTTTAATTATTTTGTGCAGCGATGGGGGCGG | |
| | GGGGGGGGGGGGGGCGCGCGCCAGGCGGGGCGG | |
| | GGCGGGGCGAGGGGCGGGGCGGGGCGAGGCGGA | |
| | GAGGTGCGGCGGCAGCCAATCAGAGCGGCGCGC | |
| | TCCGAAAGTTTCCTTTTATGGCGAGGCGGCGGC | |
| | GGCGGCGGCCCTATAAAAAGCGAAGCGCGCGGC | |
| | GGGCGGGAGTCGCTGCGACGCTGCCTTCGCCCC | |
| | GTGCCCCGCTCCGCCGCCGCCTCGCGCCGCCCG | |
| | CCCCGGCTCTGACTGACCGCGTTACTCCCACAG | |
| | GTGAGCGGGCGGGACGGCCCTTCTCCTCCGGGC | |
| | TGTAATTAGCTGAGCAAGAGGTAAGGGTTTAAG | |
| | GGATGGTTGGTTGGTGGGGTATTAATGTTTAAT | |
| | TACCTGGAGCACCTGCCTGAAATCACTTTTTTT | |
| | CAGG | |

TABLE 6
Exemplary miRNA Sponge Region Comprising Multiple
Target segments

| Sequence (SEQ ID NO) | Target segments |
|---|---|
| TTGAATGAGGCTTCAGTACTTTACAGAATCGTTGC | 1x hsa-mir-1; 2x hsa-mir- |
| CTGCACATCTTGGAAACACTTGCTGGGATTACTTC | 208a; 3x hsa-mir-208b; 3x |
| TTCAGGTTAACCCAACAGAAGGCTCGAGAAGGTAT | hsa-mir-122 |
| ATTGCTGTTGACAGTGAGCGCTACATACTTCTTTA | (as in EXG202) |
| TATGCCCATGTGAAGCCACAGATGATGGGCATATA | |
| AAGAAGTATGTATTGCCTACTGCCTCGGAATTCAA | |
| GGGGCTACTTTAGGAGCAATTATCTTGTTTACTAA | |
| AACTGAATACCTTGCTATCTCTTTGATACATTTTT | |
| ACAAAGCTGAATTAAAATGGTATAAATTAAATCAC | |
| TTTTTTCTAGTATAACCCGGGCCAAAAGCTCAGTA | |
| TAACCCGGGCCAAAAGCTCGACATAATTCGAGCAA | |
| AAAGCTAACATAATTCGAGCAAAAAGCTCTTGACA | |
| AACACCATTGTCACACTCCAACAAACACCATTGTC | |
| ACACTCCAACAAACACCATTGTCACACTCCA | |
| (SEQ ID NO: 18) | |
| GTATAACCCGGGCCAAAAGCTCAGTATAACCCGGG | 2x hsa-mir-208a; 2x hsa-mir- |
| CCAAAAGCTCGACATAATTCGAGCAAAAAGCTAAC | 208b; 3x hsa-mir-122; 3x |
| ATAATTCGAGCAAAAAGCTCTTGACAAACACCATT | hsa-mir-133a |
| GTCACACTCCAACAAACACCATTGTCACACTCCAA | (as in EXG204) |
| CAAACACCATTGTCACACTCCACCATAGACAGCTG | |
| GTTGAAGGGGACCAAAACAGCTGGTTGAAGGGGAC | |
| CAAAACAGCTGGTTGAAGGGGACCAAA (SEQ ID | |
| NO: 19) | |
| GTATAACCCGGGCCAAAAGCTCAGTATAACCCGGG | 2x hsa-mir-208a; 2x hsa-mir- |
| CCAAAAGCTCGACATAATTCGAGCAAAAAGCTAAC | 208b; 3x hsa-mir-122; 2x |
| ATAATTCGAGCAAAAAGCTCTTGACAAACACCATT | hsa-mir-488; 2x hsa-mir-1 |
| GTCACACTCCAACAAACACCATTGTCACACTCCAA | (as in EXG205) |
| CAAACACCATTGTCACACTCCACCATAGATTGAGA | |
| GTGCCATTATCTGGGATTGAGAGTGCCATTATCTG | |
| GGAATGGGCATATAAAGAAGTATGTAATGGGCATA | |
| TAAAGAAGTATGT (SEQ ID NO: 20) | |
| TTGAATGAGGCTTCAGTACTTTACAGAATCGTTGC | 1x hsa-mir-133a; 2x hsa-mir- |
| CTGCACATCTTGGAAACACTTGCTGGGATTACTTC | 208a; 2x hsa-mir-208b; 3x |
| TTCAGGTTAACCCAACAGAAGGCTCGAGAAGGTAT | hsa-mir-122 |
| ATTGCTGTTGACAGTGAGCGCTTTTGGTCCCCTTC | (as in EXG206) |
| AACCAGCTGGTGAAGCCACAGATGCAGCTGGTTGA | |
| AGGGGACCAAAATTGCCTACTGCCTCGGAATTCAA | |
| GGGGCTACTTTAGGAGCAATTATCTTGTTTACTAA | |
| AACTGAATACCTTGCTATCTCTTTGATACATTTTT | |
| ACAAAGCTGAATTAAAATGGTATAAATTAAATCAC | |
| TTTTTTCTAGTATAACCCGGGCCAAAAGCTCAGTA | |
| TAACCCGGGCCAAAAGCTCGACATAATTCGAGCAA | |
| AAAGCTAACATAATTCGAGCAAAAAGCTCTTGACA | |
| AACACCATTGTCACACTCCAACAAACACCATTGTC | |
| ACACTCCAACAAACACCATTGTCACACTCCA | |
| (SEQ ID NO: 21) | |

### 6.2. Example 2—Constructions of AAV Vectors Comprising the Nucleic Acid

Nucleic acid sequences including those described in Section 6.1 were introduced into exemplary rAAV vectors derived from AAV9 in this example to generate rAAV vectors including, for example, EXG202, EXG204, EXG205, EXG206, EXG207, EXG209, and EXG211, as shown in FIG. 1A. The nucleic acid sequences of exemplary rAAV vectors (i.e., EXG204, EXG207, EXG209 and EXG211) are shown in the table below. The target segments in these rAAV vectors are indicated in Table 6 and Table 7. The components of exemplary rAAV vectors provided herein are also provided in Table 8.

TABLE 7
Exemplary rAAV Vectors

| AAV genome name | | |
|---|---|---|
| (ITR-to-ITR) | Sequence (SEQ ID NO) | Target Segments |
| EXG204-LmiR122- | CTGCGCGCTCGCTCGCTCACTGAGGCCGC | 2x hsa-mir-208a; 2x |
| HmiR133 | CCGGGCAAAGCCCGGGCGTCGGGCGACCT | hsa-mir-208b; 3x |
| | TTGGTCGCCCGGCCTCAGTGAGCGAGCGA | hsa-mir-122; 3x hsa- |
| | GCGCGCAGAGAGGGAGTGGAATGCACGCG | mir-133a-1 |
| | TGGATCTGAGTTCAATTCACGCGTGGTAC | |
| | CCGTTACATAACTTACGGTAAATGGCCCG | |
| | CCTGGCTGACCGCCCAACGACCCCCGCCC | |
| | ATTGACGTCAATAGTAACGCCAATAGGGA | |
| | CTTTCCATTGACGTCAATGGGTGGAGTAT | |
| | TTACGGTAAACTGCCCACTTGGCAGTACA | |
| | TCAAGTGTATCATATGCCAAGTACGCCCC | |
| | CTATTGACGTCAATGACGGTAAATGGCCC | |
| | GCCTGGCATTGTGCCCAGTACATGACCTT | |
| | ATGGGACTTTCCTACTTGGCAGTACATCT | |
| | ACGTATTAGTCATCGCTATTACCATGGTC | |
| | GAGGTGAGCCCCACGTTCTGCTTCACTCT | |
| | CCCCATCTCCCCCCCCTCCCCACCCCCAA | |
| | TTTTGTATTTATTTATTTTTTAATTATTT | |
| | TGTGCAGCGATGGGGGCGGGGGGGGGGGG | |
| | GGGGCGCGCGCCAGGCGGGGCGGGGCGGG | |
| | GCGAGGGGCGGGGCGGGGCGAGGCGGAGA | |
| | GGTGCGGCGGCAGCCAATCAGAGCGGCGC | |
| | GCTCCGAAAGTTTCCTTTTATGGCGAGGC | |
| | GGCGGCGGCGGCGGCCCTATAAAAAGCGA | |
| | AGCGCGCGGCGGGCGGGAGTCGCTGCGAC | |
| | GCTGCCTTCGCCCCGTGCCCCGCTCCGCC | |
| | GCCGCCTCGCGCCGCCCGCCCCGGCTCTG | |
| | ACTGACCGCGTTACTCCCACAGGTGAGCG | |
| | GGCGGGACGGCCCTTCTCCTCCGGGCTGT | |
| | AATTAGCTGAGCAAGAGGTAAGGGTTTAA | |
| | GGGATGGTTGGTTGGTGGGGTATTAATGT | |
| | TTAATTACCTGGAGCACCTGCCTGAAATC | |
| | ACTTTTTTTCAGGAATTCCCGGGATATCG | |
| | TCGACCCACGCGTCCGGGCCCCACGCTGC | |
| | GCACCCGCGGGTTTGCTATGGCGATGAGC | |
| | AGCGGCGGCAGTGGTGGCGGCGTCCCGGA | |
| | GCAGGAGGATTCCGTGCTGTTCCGGCGCG | |
| | GCACAGGCCAGAGCGATGATTCTGACATT | |
| | TGGGATGATACAGCACTGATAAAAGCATA | |
| | TGATAAAGCTGTGGCTTCATTTAAGCATG | |
| | CTCTAAAGAATGGTGACATTTGTGAAACT | |
| | TCGGGTAAACCAAAAACCACACCTAAAAG | |
| | AAAACCTGCTAAGAAGAATAAAAGCCAAA | |
| | AGAAGAATACTGCAGCTTCCTTACAACAG | |
| | TGGAAAGTTGGGGACAAATGTTCTGCCAT | |
| | TTGGTCAGAAGACGGTTGCATTTACCCAG | |
| | CTACCATTGCTTCAATTGATTTTAAGAGA | |
| | GAAACCTGTGTTGTGGTTTACACTGGATA | |
| | TGGAAATAGAGAGGAGCAAAATCTGTCCG | |
| | ATCTACTTTCCCCAATCTGTGAAGTAGCT | |
| | AATAATATAGAACAGAATGCTCAAGAGAA | |
| | TGAAAATGAAAGCCAAGTTTCAACAGATG | |
| | AAAGTGAGAACTCCAGGTCTCCTGGAAAT | |
| | AAATCAGATAACATCAAGCCCAAATCTGC | |
| | TCCATGGAACTCTTTTCTCCCTCCACCAC | |
| | CCCCCATGCCAGGGCCAAGACTGGGACCA | |
| | GGAAAGCCAGGTCTAAAATTCAATGGCCC | |
| | ACCACCGCCACCGCCACCACCACCACCCC | |
| | ACTTACTATCATGCTGGCTGCCTCCATTT | |
| | CCTTCTGGACCACCAATAATTCCCCCACC | |
| | ACCTCCCATATGTCCAGATTCTCTTGATG | |
| | ATGCTGATGCTTTGGGAAGTATGTTAATT | |
| | TCATGGTACATGAGTGGCTATCATACTGG | |
| | CTATTATATGGGTTTTAGACAAAATCAAA | |
| | AAGAAGGAAGGTGCTCACATTCCTTAAAT | |
| | TAAGGAGAAATGCTGGCATAGAGCAGCAC | |
| | TAAATGACACCACTAAAGAAACGATCAGA | |
| | CAGATCTAGTATAACCCGGGCCAAAAGCT | |
| | CAGTATAACCCGGGCCAAAAGCTCGACAT | |
| | AATTCGAGCAAAAAGCTAACATAATTCGA | |
| | GCAAAAAGCTCTTGACAAACACCATTGTC | |
| | ACACTCCAACAAACACCATTGTCACACTC | |
| | CAACAAACACCATTGTCACACTCCACCAT | |
| | AGACAGCTGGTTGAAGGGGACCAAAACAG | |
| | CTGGTTGAAGGGGACCAAAACAGCTGGTT | |
| | GAAGGGGACCAAACAAGCTTATCGATACC | |
| | GTCGACTAGAGCTCGCTGATCAGCCTCGA | |
| | CTGTGCCTTCTAGTTGCCAGCCATCTGTT | |
| | GTTTGCCCCTCCCCCGTGCCTTCCTTGAC | |
| | CCTGGAAGGTGCCACTCCCACTGTCCTTT | |
| | CCTAATAAAATGAGGAAATTGCATCGCAT | |
| | TGTCTGAGTAGGTGTCATTCTATTCTGGG | |
| | GGGTGGGGTGGGGCAGGACAGCAAGGGGG | |
| | AGGATTGGGAAGTCTAGAGCAGGCATGCT | |
| | GGGGAGAGATCGATCTGAGGAACCCCTAG | |
| | TGATGGAGTTGGCCACTCCCTCTCTGCGC | |
| | GCTCGCTCGCTCACTGAGGCCGGGCGACC | |
| | AAAGGTCGCCCGACGCCCGGGCTTTGCCC | |
| | GGGCGGCCTCAGTGAGCGAGCGAGCGCGC | |
| | AGAGAGGGAGTGGCC (SEQ ID NO: | |
| | 22) | |
| EXG207-LmiR122- | CTGCGCGCTCGCTCGCTCACTGAGGCCGC | 2x hsa-mir-208a; 2x |
| HmiR133 | CCGGGCAAAGCCCGGGCGTCGGGCGACCT | hsa-mir-208b; 3x |
| | TTGGTCGCCCGGCCTCAGTGAGCGAGCGA | hsa-mir-122; 3x hsa- |
| | GCGCGCAGAGAGGGAGTGGAATGCACGCG | mir-133a-1 |
| | TGGATCTGAGTTCAATTCACGCGTGGTAC | |
| | CTCTGGTCGTTACATAACTTACGGTAAAT | |
| | GGCCCGCCTGGCTGACCGCCCAACGACCC | |
| | CCGCCCATTGACGTCAATAATGACGTATG | |
| | TTCCCATAGTAACGCCAATAGGGACTTTC | |
| | CATTGACGTCAATGGGTGGAGTATTTACG | |
| | GTAAACTGCCCACTTGGCAGTACATCAAG | |
| | TGTATCATATGCCAAGTACGCCCCCTATT | |
| | GACGTCAATGACGGTAAATGGCCCGCCTG | |
| | GCATTATGCCCAGTACATGACCTTATGGG | |
| | ACTTTCCTACTTGGCAGTACATCTACTCG | |
| | AGGCCACGTTCTGCTTCACTCTCCCCATC | |
| | TCCCCCCCCTCCCCACCCCCAATTTTGTA | |
| | TTTATTTATTTTTTAATTATTTTGTGCAG | |
| | CGATGGGGGCGGGGGGGGGGGGGGGGCGC | |
| | GCGCCAGGCGGGGCGGGGCGGGGCGAGGG | |
| | GCGGGGCGGGGCGAGGCGGAGAGGTGCGG | |
| | CGGCAGCCAATCAGAGCGGCGCGCTCCGA | |
| | AAGTTTCCTTTTATGGCGAGGCGGCGGCG | |
| | GCGGCGGCCCTATAAAAAGCGAAGCGCGC | |
| | GGCGGGCGGGAGCGGGATCAGCCACCGCG | |
| | GTGGCGGCCTAGAGTCGACGAGGAACTGA | |
| | AAAACCAGAAAGTTAACTGGTAAGTTTAG | |
| | TCTTTTTGTCTTTTATTTCAGGTCCCGGA | |
| | TCCGGTGGTGGTGCAAATCAAAGAACTGC | |
| | TCCTCAGTGGATGTTGCCTTTACTTCTAG | |
| | GCCTGTACGGAAGTGTTACTTCTGCTCTA | |
| | AAAGCTGCGGAATTGTACCCGCGGCCGAT | |
| | CCACCGGTCCGGAATTCCCGGGATATCGT | |
| | CGACCCACGCGTCCGGGCCCCACGCTGCG | |
| | CACCCGCGGGTTTGCTATGGCCATGAGCA | |
| | GCGGAGGAAGCGGAGGAGGAGTGCCCGAG | |
| | CAAGAGGACAGCGTGCTGTTTAGGAGAGG | |
| | AACCGGACAGAGCGATGACTCCGATATCT | |
| | GGGACGACACCGCTCTGATCAAGGCCTAT | |
| | GACAAAGCCGTGGCCTCCTTCAAGCACGC | |
| | TCTGAAGAATGGCGATATCTGTGAGACCT | |
| | CCGGCAAACCTAAGACCACCCCCAAGAGG | |
| | AAGCCCGCCAAGAAGAACAAGTCCCAGAA | |
| | GAAGAATACCGCCGCTAGCCTCCAGCAGT | |
| | GGAAAGTGGGCGATAAGTGCAGCGCCATT | |
| | TGGAGCGAGGATGGATGCATCTACCCCGC | |
| | CACCATTGCCAGCATCGACTTCAAGAGGG | |
| | AGACATGCGTGGTGGTGTATACCGGATAC | |
| | GGAAATAGAGAGGAGCAGAATCTGAGCGA | |
| | TCTGCTGTCCCCCATCTGCGAGGTGGCCA | |
| | ATAATATCGAGCAGAACGCCCAAGAGAAC | |
| | GAGAACGAAAGCCAAGTGTCCACCGATGA | |
| | GAGCGAGAACTCCAGAAGCCCCGGAAACA | |
| | AGTCCGACAACATCAAACCCAAGAGCGCC | |
| | CCTTGGAACAGCTTTCTGCCTCCTCCCCC | |
| | CCCCATGCCCGGCCCTAGACTGGGACCCG | |
| | GCAAGCCCGGACTGAAGTTCAACGGACCC | |
| | CCCCCTCCTCCTCCCCCCCCTCCTCCTCA | |
| | TCTGCTGAGCTGCTGGCTCCCCCCTTTCC | |
| | CTAGCGGCCCCCCCATTATCCCCCCCCCT | |
| | CCCCCTATCTGTCCCGACAGCCTCGATGA | |
| | CGCTGACGCCCTCGGAAGCATGCTGATCA | |
| | GCTGGTACATGAGCGGCTACCACACCGGA | |
| | TACTACATGGGCTTCAGACAGAACCAGAA | |
| | GGAGGGCAGATGCTCCCACTCTCTGAACT | |
| | GAGGAGAAATGCTGGCATAGAGCAGCACT | |
| | AAATGACACCACTAAAGAAACGATCAGAC | |
| | AGATCTAGTATAACCCGGGCCAAAAGCTC | |
| | AGTATAACCCGGGCCAAAAGCTCGACATA | |
| | ATTCGAGCAAAAAGCTAACATAATTCGAG | |
| | CAAAAAGCTCTTGACAAACACCATTGTCA | |
| | CACTCCAACAAACACCATTGTCACACTCC | |
| | AACAAACACCATTGTCACACTCCACCATA | |
| | GACAGCTGGTTGAAGGGGACCAAAACAGC | |
| | TGGTTGAAGGGGACCAAAACAGCTGGTTG | |
| | AAGGGGACCAAACAAGCTTATCGATACCG | |
| | TCGACTAGAGCTCGCTGATCAGCCTCGAC | |
| | TGTGCCTTCTAGTTGCCAGCCATCTGTTG | |
| | TTTGCCCCTCCCCCGTGCCTTCCTTGACC | |
| | CTGGAAGGTGCCACTCCCACTGTCCTTTC | |
| | CTAATAAAATGAGGAAATTGCATCGCATT | |
| | GTCTGAGTAGGTGTCATTCTATTCTGGGG | |
| | GGTGGGGTGGGGCAGGACAGCAAGGGGGA | |
| | GGATTGGGAAGTCTAGAGCAGGCATGCTG | |
| | GGGAGAGATCGATCTGAGGAACCCCTAGT | |
| | GATGGAGTTGGCCACTCCCTCTCTGCGCG | |
| | CTCGCTCGCTCACTGAGGCCGGGCGACCA | |
| | AAGGTCGCCCGACGCCCGGGCTTTGCCCG | |
| | GGCGGCCTCAGTGAGCGAGCGAGCGCGCA | |
| | GAGAGGGAGTGGCC (SEQ ID NO: | |
| | 23) | |
| EXG209-LmiR122- | CTGCGCGCTCGCTCGCTCACTGAGGCCGC | 2x hsa-mir-208a; 2x |
| HmiR133 | CCGGGCAAAGCCCGGGCGTCGGGCGACCT | hsa-mir-208b; 3x |
| | TTGGTCGCCCGGCCTCAGTGAGCGAGCGA | hsa-mir-122; 3x hsa- |
| | GCGCGCAGAGAGGGAGTGGAATTCACGCG | mir-133a-1 |
| | TGGATCTGAATTCAATTCACGCGTGGTAC | |
| | CTCTGGTCGTTACATAACTTACGGTAAAT | |
| | GGCCCGCCTGGCTGACCGCCCAACGACCC | |
| | CCGCCCATTGACGTCAATAATGACGTATG | |
| | TTCCCATAGTAACGCCAATAGGGACTTTC | |
| | CATTGACGTCAATGGGTGGAGTATTTACG | |
| | GTAAACTGCCCACTTGGCAGTACATCAAG | |
| | TGTATCATATGCCAAGTACGCCCCCTATT | |
| | GACGTCAATGACGGTAAATGGCCCGCCTG | |
| | GCATTATGCCCAGTACATGACCTTATGGG | |
| | ACTTTCCTACTTGGCAGTACATCTACTCG | |
| | AGGCCACGTTCTGCTTCACTCTCCCCATC | |
| | TCCCCCCCCTCCCCACCCCCAATTTTGTA | |
| | TTTATTTATTTTTTAATTATTTTGTGCAG | |
| | CGATGGGGGCGGGGGGGGGGGGGGGGCGC | |
| | GCGCCAGGCGGGGCGGGGCGGGGCGAGGG | |
| | GCGGGGCGGGGCGAGGCGGAGAGGTGCGG | |
| | CGGCAGCCAATCAGAGCGGCGCGCTCCGA | |
| | AAGTTTCCTTTTATGGCGAGGCGGCGGCG | |
| | GCGGCGGCCCTATAAAAAGCGAAGCGCGC | |
| | GGCGGGCGGGAGCGGGATCAGCCACCGCG | |
| | GTGGCGGCCTAGAGTCGACGAGGAACTGA | |
| | AAAACCAGAAAGTTAACTGGTAAGTTTAG | |
| | TCTTTTTGTCTTTTATTTCAGGTCCCGGA | |
| | TCCGGTGGTGGTGCAAATCAAAGAACTGC | |
| | TCCTCAGTGGATGTTGCCTTTACTTCTAG | |
| | GCCTGTACGGAAGTGTTACTTCTGCTCTA | |
| | AAAGCTGCGGAATTGTACCCGCGGCCGAT | |
| | CCACCGGTCCGGAATTCCCGGGATATCGT | |
| | CGACCCACGCGTCCGGGCCCCACGCTGCG | |
| | CACCCGCGGGTTTGCTATGGCGATGAGCA | |
| | GCGGCGGCAGTGGTGGCGGCGTCCCGGAG | |
| | CAGGAGGATTCCGTGCTGTTCCGGCGCGG | |
| | CACAGGCCAGAGCGATGATTCTGACATTT | |
| | GGGATGATACAGCACTGATAAAAGCATAT | |
| | GATAAAGCTGTGGCTTCATTTAAGCATGC | |
| | TCTAAAGAATGGTGACATTTGTGAAACTT | |
| | CGGGTAAACCAAAAACCACACCTAAAAGA | |
| | AAACCTGCTAAGAAGAATAAAAGCCAAAA | |
| | GAAGAATACTGCAGCTTCCTTACAACAGT | |
| | GGAAAGTTGGGGACAAATGTTCTGCCATT | |
| | TGGTCAGAAGACGGTTGCATTTACCCAGC | |
| | TACCATTGCTTCAATTGATTTTAAGAGAG | |
| | AAACCTGTGTTGTGGTTTACACTGGATAT | |
| | GGAAATAGAGAGGAGCAAAATCTGTCCGA | |
| | TCTACTTTCCCCAATCTGTGAAGTAGCTA | |
| | ATAATATAGAACAGAATGCTCAAGAGAAT | |
| | GAAAATGAAAGCCAAGTTTCAACAGATGA | |
| | AAGTGAGAACTCCAGGTCTCCTGGAAATA | |
| | AATCAGATAACATCAAGCCCAAATCTGCT | |
| | CCATGGAACTCTTTTCTCCCTCCACCACC | |
| | CCCCATGCCAGGGCCAAGACTGGGACCAG | |
| | GAAAGCCAGGTCTAAAATTCAATGGCCCA | |
| | CCACCGCCACCGCCACCACCACCACCCCA | |
| | CTTACTATCATGCTGGCTGCCTCCATTTC | |
| | CTTCTGGACCACCAATAATTCCCCCACCA | |
| | CCTCCCATATGTCCAGATTCTCTTGATGA | |
| | TGCTGATGCTTTGGGAAGTATGTTAATTT | |
| | CATGGTACATGAGTGGCTATCATACTGGC | |
| | TATTATATGGGTTTTAGACAAAATCAAAA | |
| | AGAAGGAAGGTGCTCACATTCCTTAAATT | |
| | AAGGAGAAATGCTGGCATAGAGCAGCACT | |
| | AAATGACACCACTAAAGAAACGATCAGAC | |
| | AGATCTAGTATAACCCGGGCCAAAAGCTC | |
| | AGTATAACCCGGGCCAAAAGCTCGACATA | |
| | ATTCGAGCAAAAAGCTAACATAATTCGAG | |
| | CAAAAAGCTCTTGACAAACACCATTGTCA | |
| | CACTCCAACAAACACCATTGTCACACTCC | |
| | AACAAACACCATTGTCACACTCCACCATA | |
| | GACAGCTGGTTGAAGGGGACCAAAACAGC | |
| | TGGTTGAAGGGGACCAAAACAGCTGGTTG | |
| | AAGGGGACCAAACAAGCTTATCGATACCG | |
| | TCGACTAGAGCTCGCTGATCAGCCTCGAC | |
| | TGTGCCTTCTAGTTGCCAGCCATCTGTTG | |
| | TTTGCCCCTCCCCCGTGCCTTCCTTGACC | |
| | CTGGAAGGTGCCACTCCCACTGTCCTTTC | |
| | CTAATAAAATGAGGAAATTGCATCGCATT | |
| | GTCTGAGTAGGTGTCATTCTATTCTGGGG | |
| | GGTGGGGTGGGGCAGGACAGCAAGGGGGA | |
| | GGATTGGGAAGTCTAGAGCAGGCATGCTG | |
| | GGGAGAGATCGATCTGAGGAACCCCTAGT | |
| | GATGGAGTTGGCCACTCCCTCTCTGCGCG | |
| | CTCGCTCGCTCACTGAGGCCGGGCGACCA | |
| | AAGGTCGCCCGACGCCCGGGCTTTGCCCG | |
| | GGCGGCCTCAGTGAGCGAGCGAGCGCGCA | |
| | GAGAGGGAGTGGCC (SEQ ID NO: | |
| | 24) | |
| EXG211-LmiR122- | CTGCGCGCTCGCTCGCTCACTGAGGCCGC | 2x hsa-mir-208a; 2x |
| HmiR133 | CCGGGCAAAGCCCGGGCGTCGGGCGACCT | hsa-mir-208b; 3x |
| | TTGGTCGCCCGGCCTCAGTGAGCGAGCGA | hsa-mir-122; 3x hsa- |
| | GCGCGCAGAGAGGGAGTGGAATGCACGCG | mir-133a-1 |
| | TGGATCTGAGTTCAATTCACGCGTGGTAC | |
| | CCGTTACATAACTTACGGTAAATGGCCCG | |
| | CCTGGCTGACCGCCCAACGACCCCCGCCC | |
| | ATTGACGTCAATAGTAACGCCAATAGGGA | |
| | CTTTCCATTGACGTCAATGGGTGGAGTAT | |
| | TTACGGTAAACTGCCCACTTGGCAGTACA | |
| | TCAAGTGTATCATATGCCAAGTACGCCCC | |
| | CTATTGACGTCAATGACGGTAAATGGCCC | |
| | GCCTGGCATTGTGCCCAGTACATGACCTT | |
| | ATGGGACTTTCCTACTTGGCAGTACATCT | |
| | ACGTATTAGTCATCGCTATTACCATGGTC | |
| | GAGGTGAGCCCCACGTTCTGCTTCACTCT | |
| | CCCCATCTCCCCCCCCTCCCCACCCCCAA | |
| | TTTTGTATTTATTTATTTTTTAATTATTT | |
| | TGTGCAGCGATGGGGGCGGGGGGGGGGGG | |
| | GGGGCGCGCGCCAGGCGGGGCGGGGCGGG | |
| | GCGAGGGGCGGGGCGGGGCGAGGCGGAGA | |
| | GGTGCGGCGGCAGCCAATCAGAGCGGCGC | |
| | GCTCCGAAAGTTTCCTTTTATGGCGAGGC | |
| | GGCGGCGGCGGCGGCCCTATAAAAAGCGA | |
| | AGCGCGCGGCGGGCGGGAGTCGCTGCGAC | |
| | GCTGCCTTCGCCCCGTGCCCCGCTCCGCC | |
| | GCCGCCTCGCGCCGCCCGCCCCGGCTCTG | |
| | ACTGACCGCGTTACTCCCACAGGTGAGCG | |
| | GGCGGGACGGCCCTTCTCCTCCGGGCTGT | |
| | AATTAGCTGAGCAAGAGGTAAGGGTTTAA | |
| | GGGATGGTTGGTTGGTGGGGTATTAATGT | |
| | TTAATTACCTGGAGCACCTGCCTGAAATC | |
| | ACTTTTTTTCAGGAATTCCCGGGATATCG | |
| | TCGACCCACGCGTCCGGGCCCCACGCTGC | |
| | GCACCCGGGGCCACCATGGCCATGAGCAG | |
| | CGGAGGAAGCGGAGGAGGAGTGCCCGAGC | |
| | AAGAGGACAGCGTGCTGTTTAGGAGAGGA | |
| | ACCGGACAGAGCGATGACTCCGATATCTG | |
| | GGACGACACCGCTCTGATCAAGGCCTATG | |
| | ACAAAGCCGTGGCCTCCTTCAAGCACGCT | |
| | CTGAAGAATGGCGATATCTGTGAGACCTC | |
| | CGGCAAACCTAAGACCACCCCCAAGAGGA | |
| | AGCCCGCCAAGAAGAACAAGTCCCAGAAG | |
| | AAGAATACCGCCGCTAGCCTCCAGCAGTG | |
| | GAAAGTGGGCGATAAGTGCAGCGCCATTT | |
| | GGAGCGAGGATGGATGCATCTACCCCGCC | |
| | ACCATTGCCAGCATCGACTTCAAGAGGGA | |
| | GACATGCGTGGTGGTGTATACCGGATACG | |
| | GAAATAGAGAGGAGCAGAATCTGAGCGAT | |
| | CTGCTGTCCCCCATCTGCGAGGTGGCCAA | |
| | TAATATCGAGCAGAACGCCCAAGAGAACG | |
| | AGAACGAAAGCCAAGTGTCCACCGATGAG | |
| | AGCGAGAACTCCAGAAGCCCCGGAAACAA | |
| | GTCCGACAACATCAAACCCAAGAGCGCCC | |
| | CTTGGAACAGCTTTCTGCCTCCTCCCCCC | |
| | CCCATGCCCGGCCCTAGACTGGGACCCGG | |
| | CAAGCCCGGACTGAAGTTCAACGGACCCC | |
| | CCCCTCCTCCTCCCCCCCCTCCTCCTCAT | |
| | CTGCTGAGCTGCTGGCTCCCCCCTTTCCC | |
| | TAGCGGCCCCCCCATTATCCCCCCCCCTC | |
| | CCCCTATCTGTCCCGACAGCCTCGATGAC | |
| | GCTGACGCCCTCGGAAGCATGCTGATCAG | |
| | CTGGTACATGAGCGGCTACCACACCGGAT | |
| | ACTACATGGGCTTCAGACAGAACCAGAAG | |
| | GAGGGCAGATGCTCCCACTCTCTGAACTG | |
| | AGGAGAAATGCTGGCATAGAGCAGCACTA | |
| | AATGACACCACTAAAGAAACGATCAGACA | |
| | GATCTAGTATAACCCGGGCCAAAAGCTCA | |
| | GTATAACCCGGGCCAAAAGCTCGACATAA | |
| | TTCGAGCAAAAAGCTAACATAATTCGAGC | |
| | AAAAAGCTCTTGACAAACACCATTGTCAC | |
| | ACTCCAACAAACACCATTGTCACACTCCA | |
| | ACAAACACCATTGTCACACTCCACCATAG | |
| | ACAGCTGGTTGAAGGGGACCAAAACAGCT | |
| | GGTTGAAGGGGACCAAAACAGCTGGTTGA | |
| | AGGGGACCAAACAAGCTTATCGATACCGT | |
| | CGACTAGAGCTCGCTGATCAGCCTCGACT | |
| | GTGCCTTCTAGTTGCCAGCCATCTGTTGT | |
| | TTGCCCCTCCCCCGTGCCTTCCTTGACCC | |
| | TGGAAGGTGCCACTCCCACTGTCCTTTCC | |
| | TAATAAAATGAGGAAATTGCATCGCATTG | |
| | TCTGAGTAGGTGTCATTCTATTCTGGGGG | |
| | GTGGGGTGGGGCAGGACAGCAAGGGGGAG | |
| | GATTGGGAAGTCTAGAGCAGGCATGCTGG | |
| | GGAGAGATCGATCTGAGGAACCCCTAGTG | |
| | ATGGAGTTGGCCACTCCCTCTCTGCGCGC | |
| | TCGCTCGCTCACTGAGGCCGGGCGACCAA | |
| | AGGTCGCCCGACGCCCGGGCTTTGCCCGG | |
| | GCGGCCTCAGTGAGCGAGCGAGCGCGCAG | |
| | AGAGGGAGTGGCC (SEQ ID NO: | |
| | 25) | |

TABLE 8
Components of Exemplary rAAV Vectors

| | SMN coding | | |
|---|---|---|---|
| | sequence | Promoter | Target Segments |
| EXG101-01 | SEQ ID NO: 34 | SEQ ID NO: 36 | — |
| EXG101-01M2 | SEQ ID NO: 34 | SEQ ID NO: 36 | — |
| EXG101-01-03 | SEQ ID NO: 35 | SEQ ID NO: 36 | — |
| EXG101-02M | SEQ ID NO: 34 | SEQ ID NO: 37 | — |
| EXG101-05M | SEQ ID NO: 35 | SEQ ID NO: 37 | — |
| EXG202 | SEQ ID NO: 34 | SEQ ID NO: 36 | 1× hsa-mir-1; |
| | | | 2× hsa-mir-208a; |
| | | | 3× hsa-mir-208b; |
| | | | 3× hsa-mir-122 |
| EXG204 | SEQ ID NO: 34 | SEQ ID NO: 36 | 2× hsa-mir-208a; |
| | | | 2× hsa-mir-208b; |
| | | | 3× hsa-mir-122; |
| | | | 3× hsa-mir- |
| | | | 133a-1 |
| EXG205 | SEQ ID NO: 34 | SEQ ID NO: 36 | 2× hsa-mir-208a; |
| | | | 2× hsa-mir-208b; |
| | | | 3× hsa-mir-122; |
| | | | 2× hsa-mir-488; |
| | | | 2× hsa-mir-1 |
| EXG206 | SEQ ID NO: 34 | SEQ ID NO: 36 | 1× hsa-mir-133a; |
| | | | 2× hsa-mir-208a; |
| | | | 2× hsa-mir-208b; |
| | | | 3× hsa-mir-122 |
| EXG207 | SEQ ID NO: 35 | SEQ ID NO: 36 | 2× hsa-mir-208a; |
| | | | 2× hsa-mir-208b; |
| | | | 3× hsa-mir-122; |
| | | | 3× hsa-mir- |
| | | | 133a-1 |
| EXG209 | SEQ ID NO: 34 | SEQ ID NO: 36 | 2× hsa-mir-208a; |
| | | | 2× hsa-mir-208b; |
| | | | 3× hsa-mir-122; |
| | | | 3× hsa-mir- |
| | | | 133a-1 |
| EXG211 | SEQ ID NO: 35 | SEQ ID NO: 37 | 2× hsa-mir-208a; |
| | | | 2× hsa-mir-208b; |
| | | | 3× hsa-mir-122; |
| | | | 3× hsa-mir- |
| | | | 133a-1 |

### 6.3. Example 3—Protein Expressions Assays

HEK 293 cells were cultured in a 12-well plate with DMEM supplemented with 10% of fetal bovine serum, and incubate in a 37° C., 5% CO₂ incubator for 24 hours before infected. The HEK 293 cells were seeded in 12-well plate with 4×10⁵ HEK 293 cells per well 24 h before infection, in 1 mL DMEM, 10% FBS, and incubated in a 37° C., 5% CO₂ incubator. The HEK 293 cells were then co-infected with rAAV-SMN (10⁵ GC/cell) provided herein and a human adeno virus 5 (Ad5) helper virus (10 iu/cell) and incubated in a 37° C., 5% CO₂ incubator for 72 h. Normal 293FT cell was used as a blank control. Cells were harvested at 72 h, washed by PBS, pelleted by centrifugation, and lysed in cell lysis buffer. Based on total protein concentrations calculated from the BCA assays, samples containing equal amounts of protein (1.25 ug/sample) were separately resolved by 4%-12% Bis-Tris gel electrophoresis, according to the manufacturer's recommendations (Invitrogen), then transferred onto nitrocellulose membrane using power Blotter station for 7 mins at 25V, and probed with the SMN antibodies and Tubulin antibody.

The rAAV vectors tested in the present example are shown in Table 8 above. For example, EXG101-01-03 comprises a promoter sequence (SEQ ID NO: 35) driven hSMN1 sequence (SEQ ID NO: 36); EXG101-05M comprises a promoter sequence (SEQ ID NO: 35) driven hSMN1 sequence (SEQ ID NO: 37); EXG101-02M comprises a promoter sequence (SEQ ID NO: 34) driven hSMN1 sequence (SEQ ID NO: 37); and EXG101-01M2 comprises a promoter sequence (SEQ ID NO: 34) driven hSMN1 sequence (SEQ ID NO: 36).

Expression intension was measured by the ratio of SMN/tubulin, and the results are shown in FIG. 2A.

### 6.4. Example 4—In Vivo Potency Assays

The constructs provided herein were tested and analyzed in in vivo potency assays by treating mouse at P0-2 age with spinal muscular atrophy (SMA) having bi-allelic mutations in the survival motor neuron 1 (smn1) gene. The in vivo potency assay refers to the rescue experiments in smn1−/− deficient mouse model, and the measurement of median survival and other relevant in vivo parameters, such as righting, body weight measurements. SMA type 1 mouse model is defined by the onset of progressive muscle weakness associated with loss of lower motor neurons prior to gaining the ability to right or rear independently.

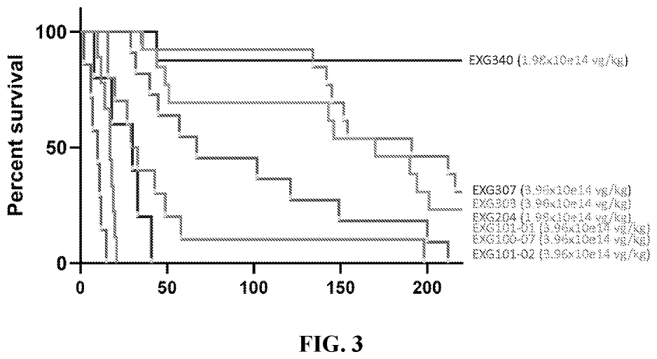

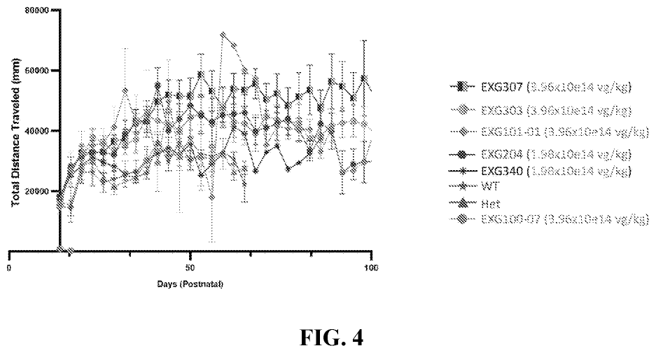

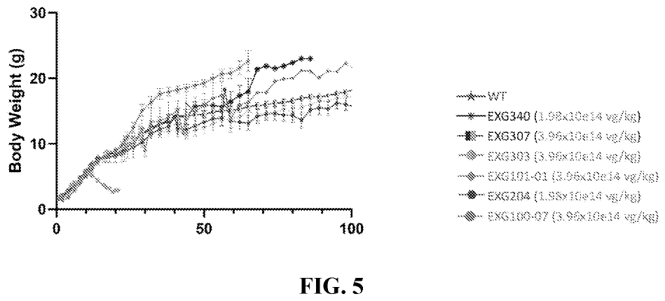

As shown in FIGS. 3-6, the mice treated with the present constructs, e.g., EXG204, demonstrated superior survival rate, comparable open field activities to control wild-type mice without treatment, and very even body weight distribution.

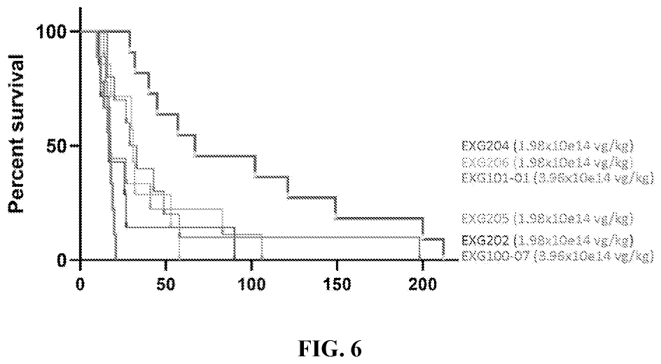

The results also demonstrate that target segments of hsa-mir-133a (e.g., as in EXG204 and EXG206) are more effective than target segments of other miRNAs, such as hsa-mir-1 and hsa-mir-488a (e.g., as in EXG202 and EXG205) (see FIG. 6). Moreover, multiple repeats of a target segment of hsa-mir-133a, e.g., 3 repeats, showed better results as compared with a single target segment of hsa-mir-133a, as exemplified by comparing EXG204 and EXG206.

In sum, the present rAAV9 viral vectors for delivery of the smn1 gene were demonstrated to express the human SMN1 protein driven by optimized promoter sequences, and simultaneously achieving (>60% better) in vivo tolerated liver toxicity and heart toxicity through post-transcriptional regulation by 3′-targeting sequences of endogenous microRNAs, and maximizing tissue specific down-regulation of SMN1 in liver and heart.

### 6.5. Example 5—Constructs Comprising Synthetic Promoters

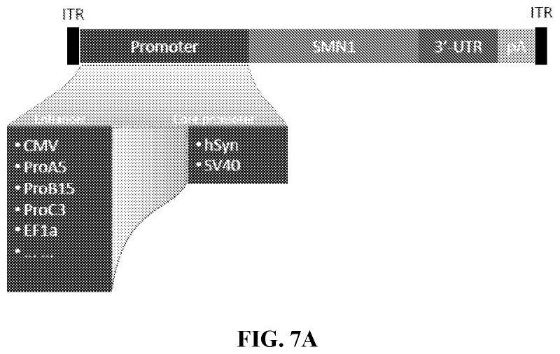

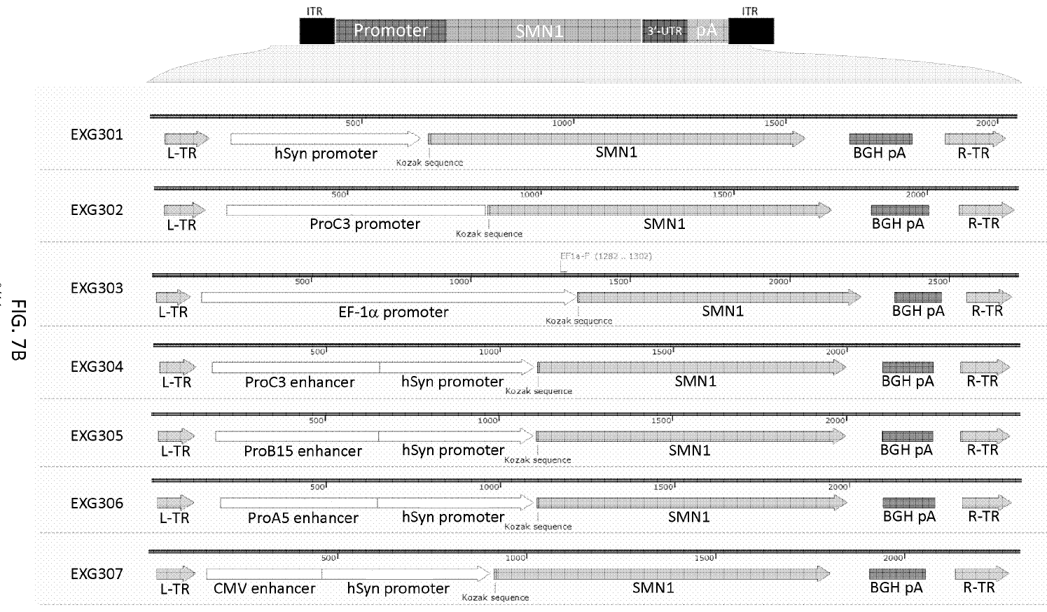

Various nucleic acid constructs comprising synthetic promoters were generated and tested in this study. Specifically, as shown in FIG. 7A and FIG. 7B, in certain constructs, various enhancers were combined with a core promoter (hSyn). For example, EXG304 comprises proC3 enhancer and hSyn promoter; EXG305 comprises proB15 enhancer and hSyn promoter; EXG306 comprises proA5 enhancer and hSyn promoter; and EXG307 comprises CMV enhancer and hSyn promoter; and EXG340 is designed by replacing the SMN1 protein coding sequence with a codon optimized sequence of human SMN1, while other elements are identical to EXG307. Various enhancers and promoters are shown in Table 9. The sequences for the rAAV vectors shown in FIG. 7B (i.e., EXG301, EXG302, EXG303, EXG304, EXG305, EXG306 and EXG307), EXG340, and EXG341, are shown in Table 10 below.

TABLE 9
Core Promoter and Enhancer Sequences

| Element name | Sequence (5′-to-3′) |
|---|---|
| Core promoter | AGTGCAAGTGGGTTTTAGGA |
| (hSyn) | CCAGGATGAGGCGGGGTGGG |
| sequences | GGTGCCTACCTGACGACCGA |
| | CCCCGACCCACTGGACAAGC |
| | ACCCAACCCCCATTCCCCAA |
| | ATTGCGCATCCCCTATCAGA |
| | GAGGGGGAGGGGAAACAGGA |
| | TGCGGCGAGGCGCGTGCGCA |
| | CTGCCAGCTTCAGCACCGCG |
| | GACAGTGCCTTCGCCCCCGC |
| | CTGGCGGCGCGCGCCACCGC |
| | CGCCTCAGCACTGAAGGCGC |
| | GCTGACGTCACTCGCCGGTC |
| | CCCCGCAAACTCCCCTTCCC |
| | GGCCACCTTGGTCGCGTCCG |
| | CGCCGCCGCCGGCCCAGCCG |
| | GACCGCACCACGCGAGGCGC |
| | GAGATAGGGGGGCACGGGCG |
| | CGACCATCTGCGCTGCGGCG |
| | CCGGCGACTCAGCGCTGCCT |
| | CAGTCTGCGGTGGGCAGCGG |
| | AGGAGTCGTGTCGTGCCTGA |
| | GAGCGCAG |
| | (SEQ ID NO: 38) |
| CMV enhancer | CGTTACATAACTTACGGTAA |
| | ATGGCCCGCCTGGCTGACCG |
| | CCCAACGACCCCCGCCCATT |
| | GACGTCAATAATGACGTATG |
| | TTCCCATAGTAACGCCAATA |
| | GGGACTTTCCATTGACGTCA |
| | ATGGGTGGAGTATTTACGGT |
| | AAACTGCCCACTTGGCAGTA |
| | CATCAAGTGTATCATATGCC |
| | AAGTACGCCCCCTATTGACG |
| | TCAATGACGGTAAATGGCCC |
| | GCCTGGCATTATGCCCAGTA |
| | CATGACCTTATGGGACTTTC |
| | CTACTTGGCAGTACATCTAC |
| | GTATTAGTCATCGCTATTAC |
| | CATG |
| | (SEQ ID NO: 39) |
| ProC3 enhancer | GATGAATTCCGCCGGAAACT |
| | AGGTCCGGAGGACTGCCGGA |
| | AACACCTGTAATCAAGCCGC |
| | CGGAAACCTGTTGTGGCCGT |
| | ATGCCGGAAACGTCTTAATT |
| | GGACGTGCCGGAAACTCTTT |
| | TAATGAGTTCGCCGGAAACC |
| | AGACCAGCCGAGCTGCCGGA |
| | AACCGGTTATATAGAACGGC |
| | CGGAAACGGTCCACAGGAAA |
| | AAGCCGGAAACACCCAAACG |
| | GTTAGCGCCGGAAACGACTG |
| | GGGAGGACGTGCCGGAAACG |
| | TACTATCTGAAGATGCCGGA |
| | AACACTTGAAAGCTCCAAGC |
| | CGGAAACGGGCCCGTGCGGA |
| | TAGCCGGAAACTGACGGTAC |
| | ACGGCCGCCGGAAACACTAC |
| | TTGTATGGTAGCCGGAAACT |
| | TGGGCGTGGCTGGGGCCGGA |
| | AACGCTCGAGATCTGCGATC |
| | (SEQ ID NO: 40) |
| ProA5 enhancer | CCTGGAGGCCTTCCTGGAAG |
| | AAGAGATCCTGGCACCGCAC |
| | AAAGAGAAGCACAGGCTTTC |
| | CAGGGCTGAGGAGAGGGAGG |
| | TCAAGTGAGGCCCAGGTGCC |
| | CCTGCCTGAGCCTGTGTCCC |
| | CAGAAACCTCCTCTCCCTCT |
| | CATCACCCCCACATCCTCCC |
| | TGCCACTCCCCGCAGCTCCC |
| | TGTGGCCAAGTGCACTGCAG |
| | CACTCGGCTCTGCTCCACAA |
| | ACGGTCTGCTCCACTCCAGG |
| | AAGGCCACCTCCTCCCCCCC |
| | CCCCCACCTCCGGCTGTCAC |
| | CACTCACCGCTCTAGCCTCC |
| | AGGGGGTGGGGACCCCAGAG |
| | CTGGACACACCCCATCGAAG |
| | CCCCACAGCTCAGCCAGCCG |
| | GACAGACTCACGGTCGGACT |
| | CAAGACCCCGGAGCCCTGAG |
| | GTGGGCAGCGCGCCAGGGTT |
| | CCTCGCAGCCTCTTCAAGGT |
| | CAGTGCAAGT |
| | (SEQ ID NO: 41) |
| ProB15 enhancer | CAGCCCCCGGGCCCTCCTCC |
| | TCCCTCTGCCTTTTTAAGGG |
| | ACGCCCTCCAGGGCGACCCC |
| | GGAGGGCGGACTTGCCAAGC |
| | TGAAGAGAATCAGTCAAAAA |
| | TCCGCCCACAGGGGACACAT |
| | CATTTAAATAAATGTGTTTC |
| | TTTGCCCGAACAGAAGTTCA |
| | GATAGGCTCGATTATCATTA |
| | ATTCTGGGTTTCACGTAACG |
| | AGAGGAAACACAGGTTGCAA |
| | TAAAAATAAAAAAATGGTTT |
| | GAAATCAATTTTAACTCATT |
| | TTGAACGTCCTCACACGTTT |
| | GACAAACCGATTTGTTTCAG |
| | GAGACTTGCTAATATCTAAA |
| | TCGGTGACAGGGTGTTTGCT |
| | GTGAGTGTGGCTCTGGAAAA |
| | GTTATTAAGCGTTATAAAAA |
| | AAATGATGTAATGAAATTCT |
| | AATTAATGGGAGGGAAGTGC |
| | CAACAAATCACTCCTTAAAA |
| | TATTAACGCTATCAAAGAAC |
| | AGCTGGAGAAGG |
| | (SEQ ID NO: 42) |
| ProC3 promoter | GAAACAGCTGAGGGTGCCCA |
| | GCCGGAAACTCGAAATCAAC |
| | GTAGGCCGGAAACTATTCGA |
| | TGAATTCCGCCGGAAACTAG |
| | GTCCGGAGGACTGCCGGAAA |
| | CACCTGTAATCAAGCCGCCG |
| | GAAACCTGTTGTGGCCGTAT |
| | GCCGGAAACGTCTTAATTGG |
| | ACGTGCCGGAAACTCTTTTA |
| | ATGAGTTCGCCGGAAACCAG |
| | ACCAGCCGAGCTGCCGGAAA |
| | CCGGTTATATAGAACGGCCG |
| | GAAACGGTCCACAGGAAAAA |
| | GCCGGAAACACCCAAACGGT |
| | TAGCGCCGGAAACGACTGGG |
| | GAGGACGTGCCGGAAACGTA |
| | CTATCTGAAGATGCCGGAAA |
| | CACTTGAAAGCTCCAAGCCG |
| | GAAACGGGCCCGTGCGGATA |
| | GCCGGAAACTGACGGTACAC |
| | GGCCGCCGGAAACACTACTT |
| | GTATGGTAGCCGGAAACTTG |
| | GGCGTGGCTGGGGCCGGAAA |
| | CGCTCGAGATCTGCGATCTG |
| | CATCTCAATTAGTCAGCAAC |
| | CATAGTCCCGCCCCTAACTC |
| | CGCCCATCCCGCCCCTAACT |
| | CCGCCCAGTTCCGCCCATTC |
| | TCCGCCCCATCGCTGACTAA |
| | TTTTTTTTATTTATGCAGAG |
| | GCCGAGGCCGCCTCGGCCTC |
| | TGAGCTATTCCAGAAGTAGT |
| | GAGGAGGCTTTTTTGGAGGC |
| | CTAGGCTTTTGCAAA |
| | (SEQ ID NO: 43) |
| EF1a promoter | GGCTCCGGTGCCCGTCAGTG |
| | GGCAGAGCGCACATCGCCCA |
| | CAGTCCCCGAGAAGTTGGGG |
| | GGAGGGGTCGGCAATTGAAC |
| | CGGTGCCTAGAGAAGGTGGC |
| | GCGGGGTAAACTGGGAAAGT |
| | GATGTCGTGTACTGGCTCCG |
| | CCTTTTTCCCGAGGGTGGGG |
| | GAGAACCGTATATAAGTGCA |
| | GTAGTCGCCGTGAACGTTCT |
| | TTTTCGCAACGGGTTTGCCG |
| | CCAGAACACAGGTAAGTGCC |
| | GTGTGTGGTTCCCGCGGGCC |
| | TGGCCTCTTTACGGGTTATG |
| | GCCCTTGCGTGCCTTGAATT |
| | ACTTCCACTGGCTGCAGTAC |
| | GTGATTCTTGATCCCGAGCT |
| | TCGGGTTGGAAGTGGGTGGG |
| | AGAGTTCGAGGCCTTGCGCT |
| | TAAGGAGCCCCTTCGCCTCG |
| | TGCTTGAGTTGAGGCCTGGC |
| | CTGGGCGCTGGGGCCGCCGC |
| | GTGCGAATCTGGTGGCACCT |
| | TCGCGCCTGTCTCGCTGCTT |
| | TCGATAAGTCTCTAGCCATT |
| | TAAAATTTTTGATGACCTGC |
| | TGCGACGCTTTTTTTCTGGC |
| | AAGATAGTCTTGTAAATGCG |
| | GGCCAAGATCTGCACACTGG |
| | TATTTCGGTTTTTGGGGCCG |
| | CGGGCGGCGACGGGGCCCGT |
| | GCGTCCCAGCGCACATGTTC |
| | GGCGAGGCGGGGCCTGCGAG |
| | CGCGGCCACCGAGAATCGGA |
| | CGGGGGTAGTCTCAAGCTGG |
| | CCGGCCTGCTCTGGTGCCTG |
| | GCCTCGCGCCGCCGTGTATC |
| | GCCCCGCCCTGGGCGGCAAG |
| | GCTGGCCCGGTCGGCACCAG |
| | TTGCGTGAGCGGAAAGATGG |
| | CCGCTTCCCGGCCCTGCTGC |
| | AGGGAGCTCAAAATGGAGGA |
| | CGCGGCGCTCGGGAGAGCGG |
| | GCGGGTGAGTCACCCACACA |
| | AAGGAAAAGGGCCTTTCCGT |
| | CCTCAGCCGTCGCTTCATGT |
| | GACTCCACGGAGTACCGGGC |
| | GCCGTCCAGGCACCTCGATT |
| | AGTTCTCGAGCTTTTGGAGT |
| | ACGTCGTCTTTAGGTTGGGG |
| | GGAGGGGTTTTATGCGATGG |
| | AGTTTCCCCACACTGAGTGG |
| | GTGGAGACTGAAGTTAGGCC |
| | AGCTTGGCACTTGATGTAAT |
| | TCTCCTTGGAATTTGCCCTT |
| | TTTGAGTTTGGATCTTGGTT |
| | CATTCTCAAGCCTCAGACAG |
| | TGGTTCAAAGTTTTTTTCTT |
| | CCATTTCAGGTGTCGTGA |
| | (SEQ ID NO: 44) |

TABLE 10
Exemplary rAAV Vectors with Various Promoters

| Construct | |
|---|---|
| names | Sequence (5′-to-3′) |
| EXG301 | CTGCGCGCTCGCTCGCTCAC |
| | TGAGGCCGCCCGGGCAAAGC |
| | CCGGGCGTCGGGCGACCTTT |
| | GGTCGCCCGGCCTCAGTGAG |
| | CGAGCGAGCGCGCAGAGAGG |
| | GAGTGGAATGCACGCGTGGA |
| | TCTGAGTTCAATTCACGCGT |
| | GCTGCGTATGGCGTATAGTG |
| | CAAGTGGGTTTTAGGACCAG |
| | GATGAGGCGGGGTGGGGGTG |
| | CCTACCTGACGACCGACCCC |
| | GACCCACTGGACAAGCACCC |
| | AACCCCCATTCCCCAAATTG |
| | CGCATCCCCTATCAGAGAGG |
| | GGGAGGGGAAACAGGATGCG |
| | GCGAGGCGCGTGCGCACTGC |
| | CAGCTTCAGCACCGCGGACA |
| | GTGCCTTCGCCCCCGCCTGG |
| | CGGCGCGCGCCACCGCCGCC |
| | TCAGCACTGAAGGCGCGCTG |
| | ACGTCACTCGCCGGTCCCCC |
| | GCAAACTCCCCTTCCCGGCC |
| | ACCTTGGTCGCGTCCGCGCC |
| | GCCGCCGGCCCAGCCGGACC |
| | GCACCACGCGAGGCGCGAGA |
| | TAGGGGGGCACGGGCGCGAC |
| | CATCTGCGCTGCGGCGCCGG |
| | CGACTCAGCGCTGCCTCAGT |
| | CTGCGGTGGGCAGCGGAGGA |
| | GTCGTGTCGTGCCTGAGAGC |
| | GCAGTCGAATTCAAGCTGCT |
| | AGCCACCATGGCGATGAGCA |
| | GCGGCGGCAGTGGTGGCGGC |
| | GTCCCGGAGCAGGAGGATTC |
| | CGTGCTGTTCCGGCGCGGCA |
| | CAGGCCAGAGCGATGATTCT |
| | GACATTTGGGATGATACAGC |
| | ACTGATAAAAGCATATGATA |
| | AAGCTGTGGCTTCATTTAAG |
| | CATGCTCTAAAGAATGGTGA |
| | CATTTGTGAAACTTCGGGTA |
| | AACCAAAAACCACACCTAAA |
| | AGAAAACCTGCTAAGAAGAA |
| | TAAAAGCCAAAAGAAGAATA |
| | CTGCAGCTTCCTTACAACAG |
| | TGGAAAGTTGGGGACAAATG |
| | TTCTGCCATTTGGTCAGAAG |
| | ACGGTTGCATTTACCCAGCT |
| | ACCATTGCTTCAATTGATTT |
| | TAAGAGAGAAACCTGTGTTG |
| | TGGTTTACACTGGATATGGA |
| | AATAGAGAGGAGCAAAATCT |
| | GTCCGATCTACTTTCCCCAA |
| | TCTGTGAAGTAGCTAATAAT |
| | ATAGAACAGAATGCTCAAGA |
| | GAATGAAAATGAAAGCCAAG |
| | TTTCAACAGATGAAAGTGAG |
| | AACTCCAGGTCTCCTGGAAA |
| | TAAATCAGATAACATCAAGC |
| | CCAAATCTGCTCCATGGAAC |
| | TCTTTTCTCCCTCCACCACC |
| | CCCCATGCCAGGGCCAAGAC |
| | TGGGACCAGGAAAGCCAGGT |
| | CTAAAATTCAATGGCCCACC |
| | ACCGCCACCGCCACCACCAC |
| | CACCCCACTTACTATCATGC |
| | TGGCTGCCTCCATTTCCTTC |
| | TGGACCACCAATAATTCCCC |
| | CACCACCTCCCATATGTCCA |
| | GATTCTCTTGATGATGCTGA |
| | TGCTTTGGGAAGTATGTTAA |
| | TTTCATGGTACATGAGTGGC |
| | TATCATACTGGCTATTATAT |
| | GGGTTTTAGACAAAATCAAA |
| | AAGAAGGAAGGTGCTCACAT |
| | TCCTTAAATTAAGGAGAAAT |
| | GCTGGCATAGAGCAGCACTA |
| | AATGACACCACTAAAGAAAC |
| | GATCAGACAGATCTACAAAG |
| | CTTATCGATACCGTCGACTA |
| | GAGCTCGCTGATCAGCCTCG |
| | ACTGTGCCTTCTAGTTGCCA |
| | GCCATCTGTTGTTTGCCCCT |
| | CCCCCGTGCCTTCCTTGACC |
| | CTGGAAGGTGCCACTCCCAC |
| | TGTCCTTTCCTAATAAAATG |
| | AGGAAATTGCATCGCATTGT |
| | CTGAGTAGGTGTCATTCTAT |
| | TCTGGGGGGTGGGGTGGGGC |
| | AGGACAGCAAGGGGGAGGAT |
| | TGGGAAGTCTAGAGCAGGCA |
| | TGCTGGGGAGAGATCGATCT |
| | GAGGAACCCCTAGTGATGGA |
| | GTTGGCCACTCCCTCTCTGC |
| | GCGCTCGCTCGCTCACTGAG |
| | GCCGGGCGACCAAAGGTCGC |
| | CCGACGCCCGGGCTTTGCCC |
| | GGGCGGCCTCAGTGAGCGAG |
| | CGAGCGCGCAGAGAGGGAGT |
| | GGCC (SEQ ID NO: 45) |
| EXG302 | CTGCGCGCTCGCTCGCTCAC |
| | TGAGGCCGCCCGGGCAAAGC |
| | CCGGGCGTCGGGCGACCTTT |
| | GGTCGCCCGGCCTCAGTGAG |
| | CGAGCGAGCGCGCAGAGAGG |
| | GAGTGGAATGCACGCGTGGA |
| | TCTGAGTTCAACGCGTAAAG |
| | CTGGAACTGGGGCCGGAAAC |
| | AGCTGAGGGTGCCCAGCCGG |
| | AAACTCGAAATCAACGTAGG |
| | CCGGAAACTATTCGATGAAT |
| | TCCGCCGGAAACTAGGTCCG |
| | GAGGACTGCCGGAAACACCT |
| | GTAATCAAGCCGCCGGAAAC |
| | CTGTTGTGGCCGTATGCCGG |
| | AAACGTCTTAATTGGACGTG |
| | CCGGAAACTCTTTTAATGAG |
| | TTCGCCGGAAACCAGACCAG |
| | CCGAGCTGCCGGAAACCGGT |
| | TATATAGAACGGCCGGAAAC |
| | GGTCCACAGGAAAAAGCCGG |
| | AAACACCCAAACGGTTAGCG |
| | CCGGAAACGACTGGGGAGGA |
| | CGTGCCGGAAACGTACTATC |
| | TGAAGATGCCGGAAACACTT |
| | GAAAGCTCCAAGCCGGAAAC |
| | GGGCCCGTGCGGATAGCCGG |
| | AAACTGACGGTACACGGCCG |
| | CCGGAAACACTACTTGTATG |
| | GTAGCCGGAAACTTGGGCGT |
| | GGCTGGGGCCGGAAACGCTC |
| | GAGATCTGCGATCTGCATCT |
| | CAATTAGTCAGCAACCATAG |
| | TCCCGCCCCTAACTCCGCCC |
| | ATCCCGCCCCTAACTCCGCC |
| | CAGTTCCGCCCATTCTCCGC |
| | CCCATCGCTGACTAATTTTT |
| | TTTATTTATGCAGAGGCCGA |
| | GGCCGCCTCGGCCTCTGAGC |
| | TATTCCAGAAGTAGTGAGGA |
| | GGCTTTTTTGGAGGCCTAGG |
| | CTTTTGCAAAGGATCCGCCA |
| | CCATGGCGATGAGCAGCGGC |
| | GGCAGTGGTGGCGGCGTCCC |
| | GGAGCAGGAGGATTCCGTGC |
| | TGTTCCGGCGCGGCACAGGC |
| | CAGAGCGATGATTCTGACAT |
| | TTGGGATGATACAGCACTGA |
| | TAAAAGCATATGATAAAGCT |
| | GTGGCTTCATTTAAGCATGC |
| | TCTAAAGAATGGTGACATTT |
| | GTGAAACTTCGGGTAAACCA |
| | AAAACCACACCTAAAAGAAA |
| | ACCTGCTAAGAAGAATAAAA |
| | GCCAAAAGAAGAATACTGCA |
| | GCTTCCTTACAACAGTGGAA |
| | AGTTGGGGACAAATGTTCTG |
| | CCATTTGGTCAGAAGACGGT |
| | TGCATTTACCCAGCTACCAT |
| | TGCTTCAATTGATTTTAAGA |
| | GAGAAACCTGTGTTGTGGTT |
| | TACACTGGATATGGAAATAG |
| | AGAGGAGCAAAATCTGTCCG |
| | ATCTACTTTCCCCAATCTGT |
| | GAAGTAGCTAATAATATAGA |
| | ACAGAATGCTCAAGAGAATG |
| | AAAATGAAAGCCAAGTTTCA |
| | ACAGATGAAAGTGAGAACTC |
| | CAGGTCTCCTGGAAATAAAT |
| | CAGATAACATCAAGCCCAAA |
| | TCTGCTCCATGGAACTCTTT |
| | TCTCCCTCCACCACCCCCCA |
| | TGCCAGGGCCAAGACTGGGA |
| | CCAGGAAAGCCAGGTCTAAA |
| | ATTCAATGGCCCACCACCGC |
| | CACCGCCACCACCACCACCC |
| | CACTTACTATCATGCTGGCT |
| | GCCTCCATTTCCTTCTGGAC |
| | CACCAATAATTCCCCCACCA |
| | CCTCCCATATGTCCAGATTC |
| | TCTTGATGATGCTGATGCTT |
| | TGGGAAGTATGTTAATTTCA |
| | TGGTACATGAGTGGCTATCA |
| | TACTGGCTATTATATGGGTT |
| | TTAGACAAAATCAAAAAGAA |
| | GGAAGGTGCTCACATTCCTT |
| | AAATTAAGGAGAAATGCTGG |
| | CATAGAGCAGCACTAAATGA |
| | CACCACTAAAGAAACGATCA |
| | GACAGATCTACAAAGCTTAT |
| | CGATACCGTCGACTAGAGCT |
| | CGCTGATCAGCCTCGACTGT |
| | GCCTTCTAGTTGCCAGCCAT |
| | CTGTTGTTTGCCCCTCCCCC |
| | GTGCCTTCCTTGACCCTGGA |
| | AGGTGCCACTCCCACTGTCC |
| | TTTCCTAATAAAATGAGGAA |
| | ATTGCATCGCATTGTCTGAG |
| | TAGGTGTCATTCTATTCTGG |
| | GGGGTGGGGTGGGGCAGGAC |
| | AGCAAGGGGGAGGATTGGGA |
| | AGTCTAGAGCAGGCATGCTG |
| | GGGAGAGATCGATCTGAGGA |
| | ACCCCTAGTGATGGAGTTGG |
| | CCACTCCCTCTCTGCGCGCT |
| | CGCTCGCTCACTGAGGCCGG |
| | GCGACCAAAGGTCGCCCGAC |
| | GCCCGGGCTTTGCCCGGGCG |
| | GCCTCAGTGAGCGAGCGAGC |
| | GCGCAGAGAGGGAGTGGCC |
| | (SEQ ID NO: 46) |
| EXG303 | CCTGCGCGCTCGCTCGCTCA |
| | CTGAGGCCGCCCGGGCAAAG |
| | CCCGGGCGTCGGGCGACCTT |
| | TGGTCGCCCGGCCTCAGTGA |
| | GCGAGCGAGCGCGCAGAGAG |
| | GGAGTGGAATGCACGCGTGG |
| | ATCTGAGTTCAATTCACGCG |
| | TGTGGCTCCGGTGCCCGTCA |
| | GTGGGCAGAGCGCACATCGC |
| | CCACAGTCCCCGAGAAGTTG |
| | GGGGGAGGGGTCGGCAATTG |
| | AACCGGTGCCTAGAGAAGGT |
| | GGCGCGGGGTAAACTGGGAA |
| | AGTGATGTCGTGTACTGGCT |
| | CCGCCTTTTTCCCGAGGGTG |
| | GGGGAGAACCGTATATAAGT |
| | GCAGTAGTCGCCGTGAACGT |
| | TCTTTTTCGCAACGGGTTTG |
| | CCGCCAGAACACAGGTAAGT |
| | GCCGTGTGTGGTTCCCGCGG |
| | GCCTGGCCTCTTTACGGGTT |
| | ATGGCCCTTGCGTGCCTTGA |
| | ATTACTTCCACTGGCTGCAG |
| | TACGTGATTCTTGATCCCGA |
| | GCTTCGGGTTGGAAGTGGGT |
| | GGGAGAGTTCGAGGCCTTGC |
| | GCTTAAGGAGCCCCTTCGCC |
| | TCGTGCTTGAGTTGAGGCCT |
| | GGCCTGGGCGCTGGGGCCGC |
| | CGCGTGCGAATCTGGTGGCA |
| | CCTTCGCGCCTGTCTCGCTG |
| | CTTTCGATAAGTCTCTAGCC |
| | ATTTAAAATTTTTGATGACC |
| | TGCTGCGACGCTTTTTTTCT |
| | GGCAAGATAGTCTTGTAAAT |
| | GCGGGCCAAGATCTGCACAC |
| | TGGTATTTCGGTTTTTGGGG |
| | CCGCGGGCGGCGACGGGGCC |
| | CGTGCGTCCCAGCGCACATG |
| | TTCGGCGAGGCGGGGCCTGC |
| | GAGCGCGGCCACCGAGAATC |
| | GGACGGGGGTAGTCTCAAGC |
| | TGGCCGGCCTGCTCTGGTGC |
| | CTGGCCTCGCGCCGCCGTGT |
| | ATCGCCCCGCCCTGGGCGGC |
| | AAGGCTGGCCCGGTCGGCAC |
| | CAGTTGCGTGAGCGGAAAGA |
| | TGGCCGCTTCCCGGCCCTGC |
| | TGCAGGGAGCTCAAAATGGA |
| | GGACGCGGCGCTCGGGAGAG |
| | CGGGCGGGTGAGTCACCCAC |
| | ACAAAGGAAAAGGGCCTTTC |
| | CGTCCTCAGCCGTCGCTTCA |
| | TGTGACTCCACGGAGTACCG |
| | GGCGCCGTCCAGGCACCTCG |
| | ATTAGTTCTCGAGCTTTTGG |
| | AGTACGTCGTCTTTAGGTTG |
| | GGGGGAGGGGTTTTATGCGA |
| | TGGAGTTTCCCCACACTGAG |
| | TGGGTGGAGACTGAAGTTAG |
| | GCCAGCTTGGCACTTGATGT |
| | AATTCTCCTTGGAATTTGCC |
| | CTTTTTGAGTTTGGATCTTG |
| | GTTCATTCTCAAGCCTCAGA |
| | CAGTGGTTCAAAGTTTTTTT |
| | CTTCCATTTCAGGTGTCGTG |
| | ACGCCACCATGGCGATGAGC |
| | AGCGGCGGCAGTGGTGGCGG |
| | CGTCCCGGAGCAGGAGGATT |
| | CCGTGCTGTTCCGGCGCGGC |
| | ACAGGCCAGAGCGATGATTC |
| | TGACATTTGGGATGATACAG |
| | CACTGATAAAAGCATATGAT |
| | AAAGCTGTGGCTTCATTTAA |
| | GCATGCTCTAAAGAATGGTG |
| | ACATTTGTGAAACTTCGGGT |
| | AAACCAAAAACCACACCTAA |
| | AAGAAAACCTGCTAAGAAGA |
| | ATAAAAGCCAAAAGAAGAAT |
| | ACTGCAGCTTCCTTACAACA |
| | GTGGAAAGTTGGGGACAAAT |
| | GTTCTGCCATTTGGTCAGAA |
| | GACGGTTGCATTTACCCAGC |
| | TACCATTGCTTCAATTGATT |
| | TTAAGAGAGAAACCTGTGTT |
| | GTGGTTTACACTGGATATGG |
| | AAATAGAGAGGAGCAAAATC |
| | TGTCCGATCTACTTTCCCCA |
| | ATCTGTGAAGTAGCTAATAA |
| | TATAGAACAGAATGCTCAAG |
| | AGAATGAAAATGAAAGCCAA |
| | GTTTCAACAGATGAAAGTGA |
| | GAACTCCAGGTCTCCTGGAA |
| | ATAAATCAGATAACATCAAG |
| | CCCAAATCTGCTCCATGGAA |
| | CTCTTTTCTCCCTCCACCAC |
| | CCCCCATGCCAGGGCCAAGA |
| | CTGGGACCAGGAAAGCCAGG |
| | TCTAAAATTCAATGGCCCAC |
| | CACCGCCACCGCCACCACCA |
| | CCACCCCACTTACTATCATG |
| | CTGGCTGCCTCCATTTCCTT |
| | CTGGACCACCAATAATTCCC |
| | CCACCACCTCCCATATGTCC |
| | AGATTCTCTTGATGATGCTG |
| | ATGCTTTGGGAAGTATGTTA |
| | ATTTCATGGTACATGAGTGG |
| | CTATCATACTGGCTATTATA |
| | TGGGTTTTAGACAAAATCAA |
| | AAAGAAGGAAGGTGCTCACA |
| | TTCCTTAAATTAAGGAGAAA |
| | TGCTGGCATAGAGCAGCACT |
| | AAATGACACCACTAAAGAAA |
| | CGATCAGACAGATCTACAAA |
| | GCTTATCGATACCGTCGACT |
| | AGAGCTCGCTGATCAGCCTC |
| | GACTGTGCCTTCTAGTTGCC |
| | AGCCATCTGTTGTTTGCCCC |
| | TCCCCCGTGCCTTCCTTGAC |
| | CCTGGAAGGTGCCACTCCCA |
| | CTGTCCTTTCCTAATAAAAT |
| | GAGGAAATTGCATCGCATTG |
| | TCTGAGTAGGTGTCATTCTA |
| | TTCTGGGGGGTGGGGTGGGG |
| | CAGGACAGCAAGGGGGAGGA |
| | TTGGGAAGTCTAGAGCAGGC |
| | ATGCTGGGGAGAGATCGATC |
| | TGAGGAACCCCTAGTGATGG |
| | AGTTGGCCACTCCCTCTCTG |
| | CGCGCTCGCTCGCTCACTGA |
| | GGCCGGGCGACCAAAGGTCG |
| | CCCGACGCCCGGGCTTTGCC |
| | CGGGCGGCCTCAGTGAGCGA |
| | GCGAGCGCGCAGAGAGGGAG |
| | TGGCC (SEQ ID NO: 47) |
| EXG304 | CTGCGCGCTCGCTCGCTCAC |
| | TGAGGCCGCCCGGGCAAAGC |
| | CCGGGCGTCGGGCGACCTTT |
| | GGTCGCCCGGCCTCAGTGAG |
| | CGAGCGAGCGCGCAGAGAGG |
| | GAGTGGAATGCACGCGTGGA |
| | TCTGAGTTCAACGCGTAAAG |
| | CTGGAACTGGGGCCGGAAAC |
| | AGCTGAGGGTGCCCAGCCGG |
| | AAACTCGAAATCAACGTAGG |
| | CCGGAAACTATTCGATGAAT |
| | TCCGCCGGAAACTAGGTCCG |
| | GAGGACTGCCGGAAACACCT |
| | GTAATCAAGCCGCCGGAAAC |
| | CTGTTGTGGCCGTATGCCGG |
| | AAACGTCTTAATTGGACGTG |
| | CCGGAAACTCTTTTAATGAG |
| | TTCGCCGGAAACCAGACCAG |
| | CCGAGCTGCCGGAAACCGGT |
| | TATATAGAACGGCCGGAAAC |
| | GGTCCACAGGAAAAAGCCGG |
| | AAACACCCAAACGGTTAGCG |
| | CCGGAAACGACTGGGGAGGA |
| | CGTGCCGGAAACGTACTATC |
| | TGAAGATGCCGGAAACACTT |
| | GAAAGCTCCAAGCCGGAAAC |
| | GGGCCCGTGCGGATAGCCGG |
| | AAACTGACGGTACACGGCCG |
| | CCGGAAACACTACTTGTATG |
| | GTAGCCGGAAACTTGGGCGT |
| | GGCTGGGGCCGGAAACGCTC |
| | GAGATCTGCGATCAGTGCAA |
| | GTGGGTTTTAGGACCAGGAT |
| | GAGGCGGGGTGGGGGTGCCT |
| | ACCTGACGACCGACCCCGAC |
| | CCACTGGACAAGCACCCAAC |
| | CCCCATTCCCCAAATTGCGC |
| | ATCCCCTATCAGAGAGGGGG |
| | AGGGGAAACAGGATGCGGCG |
| | AGGCGCGTGCGCACTGCCAG |
| | CTTCAGCACCGCGGACAGTG |
| | CCTTCGCCCCCGCCTGGCGG |
| | CGCGCGCCACCGCCGCCTCA |
| | GCACTGAAGGCGCGCTGACG |
| | TCACTCGCCGGTCCCCCGCA |
| | AACTCCCCTTCCCGGCCACC |
| | TTGGTCGCGTCCGCGCCGCC |
| | GCCGGCCCAGCCGGACCGCA |
| | CCACGCGAGGCGCGAGATAG |
| | GGGGGCACGGGCGCGACCAT |
| | CTGCGCTGCGGCGCCGGCGA |
| | CTCAGCGCTGCCTCAGTCTG |
| | CGGTGGGCAGCGGAGGAGTC |
| | GTGTCGTGCCTGAGAGCGCA |
| | GGGATACACGCCACCATGGC |
| | GATGAGCAGCGGCGGCAGTG |
| | GTGGCGGCGTCCCGGAGCAG |
| | GAGGATTCCGTGCTGTTCCG |
| | GCGCGGCACAGGCCAGAGCG |
| | ATGATTCTGACATTTGGGAT |
| | GATACAGCACTGATAAAAGC |
| | ATATGATAAAGCTGTGGCTT |
| | CATTTAAGCATGCTCTAAAG |
| | AATGGTGACATTTGTGAAAC |
| | TTCGGGTAAACCAAAAACCA |
| | CACCTAAAAGAAAACCTGCT |
| | AAGAAGAATAAAAGCCAAAA |
| | GAAGAATACTGCAGCTTCCT |
| | TACAACAGTGGAAAGTTGGG |
| | GACAAATGTTCTGCCATTTG |
| | GTCAGAAGACGGTTGCATTT |
| | ACCCAGCTACCATTGCTTCA |
| | ATTGATTTTAAGAGAGAAAC |
| | CTGTGTTGTGGTTTACACTG |
| | GATATGGAAATAGAGAGGAG |
| | CAAAATCTGTCCGATCTACT |
| | TTCCCCAATCTGTGAAGTAG |
| | CTAATAATATAGAACAGAAT |
| | GCTCAAGAGAATGAAAATGA |
| | AAGCCAAGTTTCAACAGATG |
| | AAAGTGAGAACTCCAGGTCT |
| | CCTGGAAATAAATCAGATAA |
| | CATCAAGCCCAAATCTGCTC |
| | CATGGAACTCTTTTCTCCCT |
| | CCACCACCCCCCATGCCAGG |
| | GCCAAGACTGGGACCAGGAA |
| | AGCCAGGTCTAAAATTCAAT |
| | GGCCCACCACCGCCACCGCC |
| | ACCACCACCACCCCACTTAC |
| | TATCATGCTGGCTGCCTCCA |
| | TTTCCTTCTGGACCACCAAT |
| | AATTCCCCCACCACCTCCCA |
| | TATGTCCAGATTCTCTTGAT |
| | GATGCTGATGCTTTGGGAAG |
| | TATGTTAATTTCATGGTACA |
| | TGAGTGGCTATCATACTGGC |
| | TATTATATGGGTTTTAGACA |
| | AAATCAAAAAGAAGGAAGGT |
| | GCTCACATTCCTTAAATTAA |
| | GGAGAAATGCTGGCATAGAG |
| | CAGCACTAAATGACACCACT |
| | AAAGAAACGATCAGACAGAT |
| | CTACAAAGCTTATCGATACC |
| | GTCGACTAGAGCTCGCTGAT |
| | CAGCCTCGACTGTGCCTTCT |
| | AGTTGCCAGCCATCTGTTGT |
| | TTGCCCCTCCCCCGTGCCTT |
| | CCTTGACCCTGGAAGGTGCC |
| | ACTCCCACTGTCCTTTCCTA |
| | ATAAAATGAGGAAATTGCAT |
| | CGCATTGTCTGAGTAGGTGT |
| | CATTCTATTCTGGGGGGTGG |
| | GGTGGGGCAGGACAGCAAGG |
| | GGGAGGATTGGGAAGTCTAG |
| | AGCAGGCATGCTGGGGAGAG |
| | ATCGATCTGAGGAACCCCTA |
| | GTGATGGAGTTGGCCACTCC |
| | CTCTCTGCGCGCTCGCTCGC |
| | TCACTGAGGCCGGGCGACCA |
| | AAGGTCGCCCGACGCCCGGG |
| | CTTTGCCCGGGCGGCCTCAG |
| | TGAGCGAGCGAGCGCGCAGA |
| | GAGGGAGTGGCC |
| | (SEQ ID NO: 48) |
| EXG305 | CTGCGCGCTCGCTCGCTCAC |
| | TGAGGCCGCCCGGGCAAAGC |
| | CCGGGCGTCGGGCGACCTTT |
| | GGTCGCCCGGCCTCAGTGAG |
| | CGAGCGAGCGCGCAGAGAGG |
| | GAGTGGAATGCACGCGTGGA |
| | TCTGAGTTCGCGTGCCCCTG |
| | CCTGCGCGAGGGCGGGAAGA |
| | CAGCCCCCGGGCCCTCCTCC |
| | TCCCTCTGCCTTTTTAAGGG |
| | ACGCCCTCCAGGGCGACCCC |
| | GGAGGGCGGACTTGCCAAGC |
| | TGAAGAGAATCAGTCAAAAA |
| | TCCGCCCACAGGGGACACAT |
| | CATTTAAATAAATGTGTTTC |
| | TTTGCCCGAACAGAAGTTCA |
| | GATAGGCTCGATTATCATTA |
| | ATTCTGGGTTTCACGTAACG |
| | AGAGGAAACACAGGTTGCAA |
| | TAAAAATAAAAAAATGGTTT |
| | GAAATCAATTTTAACTCATT |
| | TTGAACGTCCTCACACGTTT |
| | GACAAACCGATTTGTTTCAG |
| | GAGACTTGCTAATATCTAAA |
| | TCGGTGACAGGGTGTTTGCT |
| | GTGAGTGTGGCTCTGGAAAA |
| | GTTATTAAGCGTTATAAAAA |
| | AAATGATGTAATGAAATTCT |
| | AATTAATGGGAGGGAAGTGC |
| | CAACAAATCACTCCTTAAAA |
| | TATTAACGCTATCAAAGAAC |
| | AGCTGGAGAAGGAGTGCAAG |
| | TGGGTTTTAGGACCAGGATG |
| | AGGCGGGGTGGGGGTGCCTA |
| | CCTGACGACCGACCCCGACC |
| | CACTGGACAAGCACCCAACC |
| | CCCATTCCCCAAATTGCGCA |
| | TCCCCTATCAGAGAGGGGGA |
| | GGGGAAACAGGATGCGGCGA |
| | GGCGCGTGCGCACTGCCAGC |
| | TTCAGCACCGCGGACAGTGC |
| | CTTCGCCCCCGCCTGGCGGC |
| | GCGCGCCACCGCCGCCTCAG |
| | CACTGAAGGCGCGCTGACGT |
| | CACTCGCCGGTCCCCCGCAA |
| | ACTCCCCTTCCCGGCCACCT |
| | TGGTCGCGTCCGCGCCGCCG |
| | CCGGCCCAGCCGGACCGCAC |
| | CACGCGAGGCGCGAGATAGG |
| | GGGGCACGGGCGCGACCATC |
| | TGCGCTGCGGCGCCGGCGAC |
| | TCAGCGCTGCCTCAGTCTGC |
| | GGTGGGCAGCGGAGGAGTCG |
| | TGTCGTGCCTGAGAGCGCAG |
| | GGATACACGCCACCATGGCG |
| | ATGAGCAGCGGCGGCAGTGG |
| | TGGCGGCGTCCCGGAGCAGG |
| | AGGATTCCGTGCTGTTCCGG |
| | CGCGGCACAGGCCAGAGCGA |
| | TGATTCTGACATTTGGGATG |
| | ATACAGCACTGATAAAAGCA |
| | TATGATAAAGCTGTGGCTTC |
| | ATTTAAGCATGCTCTAAAGA |
| | ATGGTGACATTTGTGAAACT |
| | TCGGGTAAACCAAAAACCAC |
| | ACCTAAAAGAAAACCTGCTA |
| | AGAAGAATAAAAGCCAAAAG |
| | AAGAATACTGCAGCTTCCTT |
| | ACAACAGTGGAAAGTTGGGG |
| | ACAAATGTTCTGCCATTTGG |
| | TCAGAAGACGGTTGCATTTA |
| | CCCAGCTACCATTGCTTCAA |
| | TTGATTTTAAGAGAGAAACC |
| | TGTGTTGTGGTTTACACTGG |
| | ATATGGAAATAGAGAGGAGC |
| | AAAATCTGTCCGATCTACTT |
| | TCCCCAATCTGTGAAGTAGC |
| | TAATAATATAGAACAGAATG |
| | CTCAAGAGAATGAAAATGAA |
| | AGCCAAGTTTCAACAGATGA |
| | AAGTGAGAACTCCAGGTCTC |
| | CTGGAAATAAATCAGATAAC |
| | ATCAAGCCCAAATCTGCTCC |
| | ATGGAACTCTTTTCTCCCTC |
| | CACCACCCCCCATGCCAGGG |
| | CCAAGACTGGGACCAGGAAA |
| | GCCAGGTCTAAAATTCAATG |
| | GCCCACCACCGCCACCGCCA |
| | CCACCACCACCCCACTTACT |
| | ATCATGCTGGCTGCCTCCAT |
| | TTCCTTCTGGACCACCAATA |
| | ATTCCCCCACCACCTCCCAT |
| | ATGTCCAGATTCTCTTGATG |
| | ATGCTGATGCTTTGGGAAGT |
| | ATGTTAATTTCATGGTACAT |
| | GAGTGGCTATCATACTGGCT |
| | ATTATATGGGTTTTAGACAA |
| | AATCAAAAAGAAGGAAGGTG |
| | CTCACATTCCTTAAATTAAG |
| | GAGAAATGCTGGCATAGAGC |
| | AGCACTAAATGACACCACTA |
| | AAGAAACGATCAGACAGATC |
| | TACAAAGCTTATCGATACCG |
| | TCGACTAGAGCTCGCTGATC |
| | AGCCTCGACTGTGCCTTCTA |
| | GTTGCCAGCCATCTGTTGTT |
| | TGCCCCTCCCCCGTGCCTTC |
| | CTTGACCCTGGAAGGTGCCA |
| | CTCCCACTGTCCTTTCCTAA |
| | TAAAATGAGGAAATTGCATC |
| | GCATTGTCTGAGTAGGTGTC |
| | ATTCTATTCTGGGGGGTGGG |
| | GTGGGGCAGGACAGCAAGGG |
| | GGAGGATTGGGAAGTCTAGA |
| | GCAGGCATGCTGGGGAGAGA |
| | TCGATCTGAGGAACCCCTAG |
| | TGATGGAGTTGGCCACTCCC |
| | TCTCTGCGCGCTCGCTCGCT |
| | CACTGAGGCCGGGCGACCAA |
| | AGGTCGCCCGACGCCCGGGC |
| | TTTGCCCGGGCGGCCTCAGT |
| | GAGCGAGCGAGCGCGCAGAG |
| | AGGGAGTGGCC |
| | (SEQ ID NO: 49) |
| EXG306 | CTGCGCGCTCGCTCGCTCAC |
| | TGAGGCCGCCCGGGCAAAGC |
| | CCGGGCGTCGGGCGACCTTT |
| | GGTCGCCCGGCCTCAGTGAG |
| | CGAGCGAGCGCGCAGAGAGG |
| | GAGTGGAATGCACGCGTGGA |
| | TCTGAGTTCGCGTGGTGCCC |
| | AGGCAGTGGGAGCAGGGCTG |
| | ACCAGAGTTCTGCAGAGATT |
| | GCCTGGAGGCCTTCCTGGAA |
| | GAAGAGATCCTGGCACCGCA |
| | CAAAGAGAAGCACAGGCTTT |
| | CCAGGGCTGAGGAGAGGGAG |
| | GTCAAGTGAGGCCCAGGTGC |
| | CCCTGCCTGAGCCTGTGTCC |
| | CCAGAAACCTCCTCTCCCTC |
| | TCATCACCCCCACATCCTCC |
| | CTGCCACTCCCCGCAGCTCC |
| | CTGTGGCCAAGTGCACTGCA |
| | GCACTCGGCTCTGCTCCACA |
| | AACGGTCTGCTCCACTCCAG |
| | GAAGGCCACCTCCTCCCCCC |
| | CCCCCCACCTCCGGCTGTCA |
| | CCACTCACCGCTCTAGCCTC |
| | CAGGGGGTGGGGACCCCAGA |
| | GCTGGACACACCCCATCGAA |
| | GCCCCACAGCTCAGCCAGCC |
| | GGACAGACTCACGGTCGGAC |
| | TCAAGACCCCGGAGCCCTGA |
| | GGTGGGCAGCGCGCCAGGGT |
| | TCCTCGCAGCCTCTTCAAGG |
| | TCAGTGCAAGTAGTGCAAGT |
| | GGGTTTTAGGACCAGGATGA |
| | GGCGGGGTGGGGGTGCCTAC |
| | CTGACGACCGACCCCGACCC |
| | ACTGGACAAGCACCCAACCC |
| | CCATTCCCCAAATTGCGCAT |
| | CCCCTATCAGAGAGGGGGAG |
| | GGGAAACAGGATGCGGCGAG |
| | GCGCGTGCGCACTGCCAGCT |
| | TCAGCACCGCGGACAGTGCC |
| | TTCGCCCCCGCCTGGCGGCG |
| | CGCGCCACCGCCGCCTCAGC |
| | ACTGAAGGCGCGCTGACGTC |
| | ACTCGCCGGTCCCCCGCAAA |
| | CTCCCCTTCCCGGCCACCTT |
| | GGTCGCGTCCGCGCCGCCGC |
| | CGGCCCAGCCGGACCGCACC |
| | ACGCGAGGCGCGAGATAGGG |
| | GGGCACGGGCGCGACCATCT |
| | GCGCTGCGGCGCCGGCGACT |
| | CAGCGCTGCCTCAGTCTGCG |
| | GTGGGCAGCGGAGGAGTCGT |
| | GTCGTGCCTGAGAGCGCAGG |
| | GATACACGCCACCATGGCGA |
| | TGAGCAGCGGCGGCAGTGGT |
| | GGCGGCGTCCCGGAGCAGGA |
| | GGATTCCGTGCTGTTCCGGC |
| | GCGGCACAGGCCAGAGCGAT |
| | GATTCTGACATTTGGGATGA |
| | TACAGCACTGATAAAAGCAT |
| | ATGATAAAGCTGTGGCTTCA |
| | TTTAAGCATGCTCTAAAGAA |
| | TGGTGACATTTGTGAAACTT |
| | CGGGTAAACCAAAAACCACA |
| | CCTAAAAGAAAACCTGCTAA |
| | GAAGAATAAAAGCCAAAAGA |
| | AGAATACTGCAGCTTCCTTA |
| | CAACAGTGGAAAGTTGGGGA |
| | CAAATGTTCTGCCATTTGGT |
| | CAGAAGACGGTTGCATTTAC |
| | CCAGCTACCATTGCTTCAAT |
| | TGATTTTAAGAGAGAAACCT |
| | GTGTTGTGGTTTACACTGGA |
| | TATGGAAATAGAGAGGAGCA |
| | AAATCTGTCCGATCTACTTT |
| | CCCCAATCTGTGAAGTAGCT |
| | AATAATATAGAACAGAATGC |
| | TCAAGAGAATGAAAATGAAA |
| | GCCAAGTTTCAACAGATGAA |
| | AGTGAGAACTCCAGGTCTCC |
| | TGGAAATAAATCAGATAACA |
| | TCAAGCCCAAATCTGCTCCA |
| | TGGAACTCTTTTCTCCCTCC |
| | ACCACCCCCCATGCCAGGGC |
| | CAAGACTGGGACCAGGAAAG |
| | CCAGGTCTAAAATTCAATGG |
| | CCCACCACCGCCACCGCCAC |
| | CACCACCACCCCACTTACTA |
| | TCATGCTGGCTGCCTCCATT |
| | TCCTTCTGGACCACCAATAA |
| | TTCCCCCACCACCTCCCATA |
| | TGTCCAGATTCTCTTGATGA |
| | TGCTGATGCTTTGGGAAGTA |
| | TGTTAATTTCATGGTACATG |
| | AGTGGCTATCATACTGGCTA |
| | TTATATGGGTTTTAGACAAA |
| | ATCAAAAAGAAGGAAGGTGC |
| | TCACATTCCTTAAATTAAGG |
| | AGAAATGCTGGCATAGAGCA |
| | GCACTAAATGACACCACTAA |
| | AGAAACGATCAGACAGATCT |
| | ACAAAGCTTATCGATACCGT |
| | CGACTAGAGCTCGCTGATCA |
| | GCCTCGACTGTGCCTTCTAG |
| | TTGCCAGCCATCTGTTGTTT |
| | GCCCCTCCCCCGTGCCTTCC |
| | TTGACCCTGGAAGGTGCCAC |
| | TCCCACTGTCCTTTCCTAAT |
| | AAAATGAGGAAATTGCATCG |
| | CATTGTCTGAGTAGGTGTCA |
| | TTCTATTCTGGGGGGTGGGG |
| | TGGGGCAGGACAGCAAGGGG |
| | GAGGATTGGGAAGTCTAGAG |
| | CAGGCATGCTGGGGAGAGAT |
| | CGATCTGAGGAACCCCTAGT |
| | GATGGAGTTGGCCACTCCCT |
| | CTCTGCGCGCTCGCTCGCTC |
| | ACTGAGGCCGGGCGACCAAA |
| | GGTCGCCCGACGCCCGGGCT |
| | TTGCCCGGGCGGCCTCAGTG |
| | AGCGAGCGAGCGCGCAGAGA |
| | GGGAGTGGCC |
| | (SEQ ID NO: 50) |
| EXG307 | CTGCGCGCTCGCTCGCTCAC |
| | TGAGGCCGCCCGGGCAAAGC |
| | CCGGGCGTCGGGCGACCTTT |
| | GGTCGCCCGGCCTCAGTGAG |
| | CGAGCGAGCGCGCAGAGAGG |
| | GAGTGGAATGCACGCGTGGA |
| | TCTGAGTTCGCGTCGTTACA |
| | TAACTTACGGTAAATGGCCC |
| | GCCTGGCTGACCGCCCAACG |
| | ACCCCCGCCCATTGACGTCA |
| | ATAATGACGTATGTTCCCAT |
| | AGTAACGCCAATAGGGACTT |
| | TCCATTGACGTCAATGGGTG |
| | GAGTATTTACGGTAAACTGC |
| | CCACTTGGCAGTACATCAAG |
| | TGTATCATATGCCAAGTACG |
| | CCCCCTATTGACGTCAATGA |
| | CGGTAAATGGCCCGCCTGGC |
| | ATTATGCCCAGTACATGACC |
| | TTATGGGACTTTCCTACTTG |
| | GCAGTACATCTACGTATTAG |
| | TCATCGCTATTACCATGAGT |
| | GCAAGTGGGTTTTAGGACCA |
| | GGATGAGGCGGGGTGGGGGT |
| | GCCTACCTGACGACCGACCC |
| | CGACCCACTGGACAAGCACC |
| | CAACCCCCATTCCCCAAATT |
| | GCGCATCCCCTATCAGAGAG |
| | GGGGAGGGGAAACAGGATGC |
| | GGCGAGGCGCGTGCGCACTG |
| | CCAGCTTCAGCACCGCGGAC |
| | AGTGCCTTCGCCCCCGCCTG |
| | GCGGCGCGCGCCACCGCCGC |
| | CTCAGCACTGAAGGCGCGCT |
| | GACGTCACTCGCCGGTCCCC |
| | CGCAAACTCCCCTTCCCGGC |
| | CACCTTGGTCGCGTCCGCGC |
| | CGCCGCCGGCCCAGCCGGAC |
| | CGCACCACGCGAGGCGCGAG |
| | ATAGGGGGGCACGGGCGCGA |
| | CCATCTGCGCTGCGGCGCCG |
| | GCGACTCAGCGCTGCCTCAG |
| | TCTGCGGTGGGCAGCGGAGG |
| | AGTCGTGTCGTGCCTGAGAG |
| | CGCAGGGATACACGCCACCA |
| | TGGCGATGAGCAGCGGCGGC |
| | AGTGGTGGCGGCGTCCCGGA |
| | GCAGGAGGATTCCGTGCTGT |
| | TCCGGCGCGGCACAGGCCAG |
| | AGCGATGATTCTGACATTTG |
| | GGATGATACAGCACTGATAA |
| | AAGCATATGATAAAGCTGTG |
| | GCTTCATTTAAGCATGCTCT |
| | AAAGAATGGTGACATTTGTG |
| | AAACTTCGGGTAAACCAAAA |
| | ACCACACCTAAAAGAAAACC |
| | TGCTAAGAAGAATAAAAGCC |
| | AAAAGAAGAATACTGCAGCT |
| | TCCTTACAACAGTGGAAAGT |
| | TGGGGACAAATGTTCTGCCA |
| | TTTGGTCAGAAGACGGTTGC |
| | ATTTACCCAGCTACCATTGC |
| | TTCAATTGATTTTAAGAGAG |
| | AAACCTGTGTTGTGGTTTAC |
| | ACTGGATATGGAAATAGAGA |
| | GGAGCAAAATCTGTCCGATC |
| | TACTTTCCCCAATCTGTGAA |
| | GTAGCTAATAATATAGAACA |
| | GAATGCTCAAGAGAATGAAA |
| | ATGAAAGCCAAGTTTCAACA |
| | GATGAAAGTGAGAACTCCAG |
| | GTCTCCTGGAAATAAATCAG |
| | ATAACATCAAGCCCAAATCT |
| | GCTCCATGGAACTCTTTTCT |
| | CCCTCCACCACCCCCCATGC |
| | CAGGGCCAAGACTGGGACCA |
| | GGAAAGCCAGGTCTAAAATT |
| | CAATGGCCCACCACCGCCAC |
| | CGCCACCACCACCACCCCAC |
| | TTACTATCATGCTGGCTGCC |
| | TCCATTTCCTTCTGGACCAC |
| | CAATAATTCCCCCACCACCT |
| | CCCATATGTCCAGATTCTCT |
| | TGATGATGCTGATGCTTTGG |
| | GAAGTATGTTAATTTCATGG |
| | TACATGAGTGGCTATCATAC |
| | TGGCTATTATATGGGTTTTA |
| | GACAAAATCAAAAAGAAGGA |
| | AGGTGCTCACATTCCTTAAA |
| | TTAAGGAGAAATGCTGGCAT |
| | AGAGCAGCACTAAATGACAC |
| | CACTAAAGAAACGATCAGAC |
| | AGATCTACAAAGCTTATCGA |
| | TACCGTCGACTAGAGCTCGC |
| | TGATCAGCCTCGACTGTGCC |
| | TTCTAGTTGCCAGCCATCTG |
| | TTGTTTGCCCCTCCCCCGTG |
| | CCTTCCTTGACCCTGGAAGG |
| | TGCCACTCCCACTGTCCTTT |
| | CCTAATAAAATGAGGAAATT |
| | GCATCGCATTGTCTGAGTAG |
| | GTGTCATTCTATTCTGGGGG |
| | GTGGGGTGGGGCAGGACAGC |
| | AAGGGGGAGGATTGGGAAGT |
| | CTAGAGCAGGCATGCTGGGG |
| | AGAGATCGATCTGAGGAACC |
| | CCTAGTGATGGAGTTGGCCA |
| | CTCCCTCTCTGCGCGCTCGC |
| | TCGCTCACTGAGGCCGGGCG |
| | ACCAAAGGTCGCCCGACGCC |
| | CGGGCTTTGCCCGGGCGGCC |
| | TCAGTGAGCGAGCGAGCGCG |
| | CAGAGAGGGAGTGGCC |
| | (SEQ ID NO: 51) |
| EXG340 | CTGCGCGCTCGCTCGCTCAC |
| | TGAGGCCGCCCGGGCAAAGC |
| | CCGGGCGTCGGGCGACCTTT |
| | GGTCGCCCGGCCTCAGTGAG |
| | CGAGCGAGCGCGCAGAGAGG |
| | GAGTGGAATGCACGCGTGGA |
| | TCTGAGTTCGCGTCGTTACA |
| | TAACTTACGGTAAATGGCCC |
| | GCCTGGCTGACCGCCCAACG |
| | ACCCCCGCCCATTGACGTCA |
| | ATAATGACGTATGTTCCCAT |
| | AGTAACGCCAATAGGGACTT |
| | TCCATTGACGTCAATGGGTG |
| | GAGTATTTACGGTAAACTGC |
| | CCACTTGGCAGTACATCAAG |
| | TGTATCATATGCCAAGTACG |
| | CCCCCTATTGACGTCAATGA |
| | CGGTAAATGGCCCGCCTGGC |
| | ATTATGCCCAGTACATGACC |
| | TTATGGGACTTTCCTACTTG |
| | GCAGTACATCTACGTATTAG |
| | TCATCGCTATTACCATGAGT |
| | GCAAGTGGGTTTTAGGACCA |
| | GGATGAGGCGGGGTGGGGGT |
| | GCCTACCTGACGACCGACCC |
| | CGACCCACTGGACAAGCACC |
| | CAACCCCCATTCCCCAAATT |
| | GCGCATCCCCTATCAGAGAG |
| | GGGGAGGGGAAACAGGATGC |
| | GGCGAGGCGCGTGCGCACTG |
| | CCAGCTTCAGCACCGCGGAC |
| | AGTGCCTTCGCCCCCGCCTG |
| | GCGGCGCGCGCCACCGCCGC |
| | CTCAGCACTGAAGGCGCGCT |
| | GACGTCACTCGCCGGTCCCC |
| | CGCAAACTCCCCTTCCCGGC |
| | CACCTTGGTCGCGTCCGCGC |
| | CGCCGCCGGCCCAGCCGGAC |
| | CGCACCACGCGAGGCGCGAG |
| | ATAGGGGGGCACGGGCGCGA |
| | CCATCTGCGCTGCGGCGCCG |
| | GCGACTCAGCGCTGCCTCAG |
| | TCTGCGGTGGGCAGCGGAGG |
| | AGTCGTGTCGTGCCTGAGAG |
| | CGCAGGGATACACGCCACCA |
| | TGGCCATGAGCAGCGGAGGA |
| | AGCGGAGGAGGAGTGCCCGA |
| | GCAAGAGGACAGCGTGCTGT |
| | TTAGGAGAGGAACCGGACAG |
| | AGCGATGACTCCGATATCTG |
| | GGACGACACCGCTCTGATCA |
| | AGGCCTATGACAAAGCCGTG |
| | GCCTCCTTCAAGCACGCTCT |
| | GAAGAATGGCGATATCTGTG |
| | AGACCTCCGGCAAACCTAAG |
| | ACCACCCCCAAGAGGAAGCC |
| | CGCCAAGAAGAACAAGTCCC |
| | AGAAGAAGAATACCGCCGCT |
| | AGCCTCCAGCAGTGGAAAGT |
| | GGGCGATAAGTGCAGCGCCA |
| | TTTGGAGCGAGGATGGATGC |
| | ATCTACCCCGCCACCATTGC |
| | CAGCATCGACTTCAAGAGGG |
| | AGACATGCGTGGTGGTGTAT |
| | ACCGGATACGGAAATAGAGA |
| | GGAGCAGAATCTGAGCGATC |
| | TGCTGTCCCCCATCTGCGAG |
| | GTGGCCAATAATATCGAGCA |
| | GAACGCCCAAGAGAACGAGA |
| | ACGAAAGCCAAGTGTCCACC |
| | GATGAGAGCGAGAACTCCAG |
| | AAGCCCCGGAAACAAGTCCG |
| | ACAACATCAAACCCAAGAGC |
| | GCCCCTTGGAACAGCTTTCT |
| | GCCTCCTCCCCCCCCCATGC |
| | CCGGCCCTAGACTGGGACCC |
| | GGCAAGCCCGGACTGAAGTT |
| | CAACGGACCCCCCCCTCCTC |
| | CTCCCCCCCCTCCTCCTCAT |
| | CTGCTGAGCTGCTGGCTCCC |
| | CCCTTTCCCTAGCGGCCCCC |
| | CCATTATCCCCCCCCCTCCC |
| | CCTATCTGTCCCGACAGCCT |
| | CGATGACGCTGACGCCCTCG |
| | GAAGCATGCTGATCAGCTGG |
| | TACATGAGCGGCTACCACAC |
| | CGGATACTACATGGGCTTCA |
| | GACAGAACCAGAAGGAGGGC |
| | AGATGCTCCCACTCTCTGAA |
| | CTGAGGAGAAATGCTGGCAT |
| | AGAGCAGCACTAAATGACAC |
| | CACTAAAGAAACGATCAGAC |
| | AGATCTACAAAGCTTATCGA |
| | TACCGTCGACTAGAGCTCGC |
| | TGATCAGCCTCGACTGTGCC |
| | TTCTAGTTGCCAGCCATCTG |
| | TTGTTTGCCCCTCCCCCGTG |
| | CCTTCCTTGACCCTGGAAGG |
| | TGCCACTCCCACTGTCCTTT |
| | CCTAATAAAATGAGGAAATT |
| | GCATCGCATTGTCTGAGTAG |
| | GTGTCATTCTATTCTGGGGG |
| | GTGGGGTGGGGCAGGACAGC |
| | AAGGGGGAGGATTGGGAAGT |
| | CTAGAGCAGGCATGCTGGGG |
| | AGAGATCGATCTGAGGAACC |
| | CCTAGTGATGGAGTTGGCCA |
| | CTCCCTCTCTGCGCGCTCGC |
| | TCGCTCACTGAGGCCGGGCG |
| | ACCAAAGGTCGCCCGACGCC |
| | CGGGCTTTGCCCGGGCGGCC |
| | TCAGTGAGCGAGCGAGCGCG |
| | CAGAGAGGGAGTGGCC |
| | (SEQ ID NO: 52) |
| EXG341 | CTGCGCGCTCGCTCGCTCAC |
| | TGAGGCCGCCCGGGCAAAGC |
| | CCG |
| | GGCGTCGGGCGACCTTTGGT |
| | CGCCCGGCCTCAGTGAGCGA |
| | GCGAGCGCGCAGAGAGGGAG |
| | TGGAATGCACGCGTGGATCT |
| | GAGTTCGCGTCGTTACATAA |
| | CTTACGGTAAATGGCCCGCC |
| | TGGCTGACCGCCCAACGACC |
| | CCCGCCCATTGACGTCAATA |
| | ATGACGTATGTTCCCATAGT |
| | AACGCCAATAGGGACTTTCC |
| | ATTGACGTCAATGGGTGGAG |
| | TATTTACGGTAAACTGCCCA |
| | CTTGGCAGTACATCAAGTGT |
| | ATCATATGCCAAGTACGCCC |
| | CCTATTGACGTCAATGACGG |
| | TAAATGGCCCGCCTGGCATT |
| | ATGCCCAGTACATGACCTTA |
| | TGGGACTTTCCTACTTGGCA |
| | GTACATCTACGTATTAGTCA |
| | TCGCTATTACCATGAGTGCA |
| | AGTGGGTTTTAGGACCAGGA |
| | TGAGGCGGGGTGGGGGTGCC |
| | TACCTGACGACCGACCCCGA |
| | CCCACTGGACAAGCACCCAA |
| | CCCCCATTCCCCAAATTGCG |
| | CATCCCCTATCAGAGAGGGG |
| | GAGGGGAAACAGGATGCGGC |
| | GAGGCGCGTGCGCACTGCCA |
| | GCTTCAGCACCGCGGACAGT |
| | GCCTTCGCCCCCGCCTGGCG |
| | GCGCGCGCCACCGCCGCCTC |
| | AGCACTGAAGGCGCGCTGAC |
| | GTCACTCGCCGGTCCCCCGC |
| | AAACTCCCCTTCCCGGCCAC |
| | CTTGGTCGCGTCCGCGCCGC |
| | CGCCGGCCCAGCCGGACCGC |
| | ACCACGCGAGGCGCGAGATA |
| | GGGGGGCACGGGCGCGACCA |
| | TCTGCGCTGCGGCGCCGGCG |
| | ACTCAGCGCTGCCTCAGTCT |
| | GCGGTGGGCAGCGGAGGAGT |
| | CGTGTCGTGCCTGAGAGCGC |
| | AGGGATACACGCCACCATGG |
| | CCATGAGCAGCGGAGGAAGC |
| | GGAGGAGGAGTGCCCGAGCA |
| | AGAGGACAGCGTGCTGTTTA |
| | GGAGAGGAACCGGACAGAGC |
| | GATGACTCCGATATCTGGGA |
| | CGACACCGCTCTGATCAAGG |
| | CCTATGACAAAGCCGTGGCC |
| | TCCTTCAAGCACGCTCTGAA |
| | GAATGGCGATATCTGTGAGA |
| | CCTCCGGCAAACCTAAGACC |
| | ACCCCCAAGAGGAAGCCCGC |
| | CAAGAAGAACAAGTCCCAGA |
| | AGAAGAATACCGCCGCTAGC |
| | CTCCAGCAGTGGAAAGTGGG |
| | CGATAAGTGCAGCGCCATTT |
| | GGAGCGAGGATGGATGCATC |
| | TACCCCGCCACCATTGCCAG |
| | CATCGACTTCAAGAGGGAGA |
| | CATGCGTGGTGGTGTATACC |
| | GGATACGGAAATAGAGAGGA |
| | GCAGAATCTGAGCGATCTGC |
| | TGTCCCCCATCTGCGAGGTG |
| | GCCAATAATATCGAGCAGAA |
| | CGCCCAAGAGAACGAGAACG |
| | AAAGCCAAGTGTCCACCGAT |
| | GAGAGCGAGAACTCCAGAAG |
| | CCCCGGAAACAAGTCCGACA |
| | ACATCAAACCCAAGAGCGCC |
| | CCTTGGAACAGCTTTCTGCC |
| | TCCTCCCCCCCCCATGCCCG |
| | GCCCTAGACTGGGACCCGGC |
| | AAGCCCGGACTGAAGTTCAA |
| | CGGACCCCCCCCTCCTCCTC |
| | CCCCCCCTCCTCCTCATCTG |
| | CTGAGCTGCTGGCTCCCCCC |
| | TTTCCCTAGCGGCCCCCCCA |
| | TTATCCCCCCCCCTCCCCCT |
| | ATCTGTCCCGACAGCCTCGA |
| | TGACGCTGACGCCCTCGGAA |
| | GCATGCTGATCAGCTGGTAC |
| | ATGAGCGGCTACCACACCGG |
| | ATACTACATGGGCTTCAGAC |
| | AGAACCAGAAGGAGGGCAGA |
| | TGCTCCCACTCTCTGAACTG |
| | AGGAGAAATGCTGGCATAGA |
| | GCAGCACTAAATGACACCAC |
| | TAAAGAAACGATCAGACAGA |
| | TCTATAATCAACCTCTGGAT |
| | TACAAAATTTGTGAAAGATT |
| | GACTGGTATTCTTAACTATG |
| | TTGCTCCTTTTACGCTATGT |
| | GGATACGCTGCTTTAATGCC |
| | TTTGTATCATGCTATTGCTT |
| | CCCGTATGGCTTTCATTTTC |
| | TCCTCCTTGTATAAATCCTG |
| | GTTAGTTCTTGCCACGGCGG |
| | AACTCATCGCCGCCTGCCTT |
| | GCCCGCTGCTGGACAGGGGC |
| | TCGGCTGTTGGGCACTGACA |
| | ATTCCGTGGTGTTAAGCTTA |
| | TCGATACCGTCGACTAGAGC |
| | TCGCTGATCAGCCTCGACTG |
| | TGCCTTCTAGTTGCCAGCCA |
| | TCTGTTGTTTGCCCCTCCCC |
| | CGTGCCTTCCTTGACCCTGG |
| | AAGGTGCCACTCCCACTGTC |
| | CTTTCCTAATAAAATGAGGA |
| | AATTGCATCGCATTGTCTGA |
| | GTAGGTGTCATTCTATTCTG |
| | GGGGGTGGGGTGGGGCAGGA |
| | CAGCAAGGGGGAGGATTGGG |
| | AAGTCTAGAGCAGGCATGCT |
| | GGGGAGAGATCGATCTGAGG |
| | AACCCCTAGTGATGGAGTTG |
| | GCCACTCCCTCTCTGCGCGC |
| | TCGCTCGCTCACTGAGGCCG |
| | GGCGACCAAAGGTCGCCCGA |
| | CGCCCGGGCTTTGCCCGGGC |
| | GGCCTCAGTGAGCGAGCGAG |
| | CGCGCAGAGAGGGAGTGGCC |
| | (SEQ ID NO: 53) |

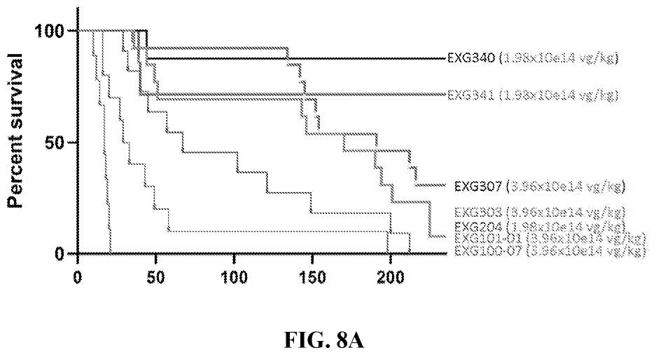

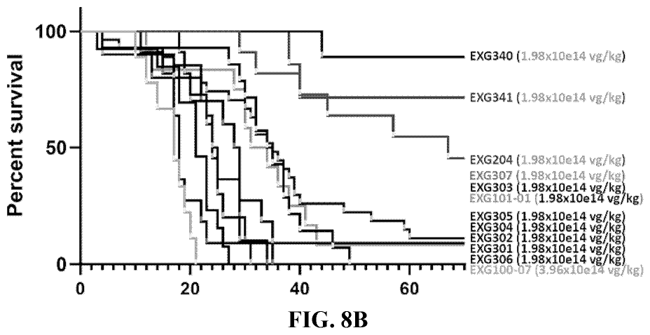

The in vivo potency assay described in Example 4 was used to examine the various constructs with different promoters described above with a single IV administration at dose level of 1.98×10¹⁴ or 3.96×10¹⁴ vg/kg. As shown in FIG. 8A and FIG. 8B, EXG303, EXG307, and EXG340 showed dose-dependent improvement in survival. Although all the EXG candidates consistently demonstrated therapeutic effects compared to GFP control—EXG100-07, EXG-307 performed significantly better than EXG301, EXG204 and EXG101-01. Surprisingly, when constructs with different synthetic promoters were adminstered as a single IV administration at dose level of 3.96×10¹⁴ vg/kg, synthetic promoter containing construct EXG307 and EXG340 demonstrated significant improvement in survival compared to other promoters including other synthetic promoters, such as EXG-304, EXG-305, and EXG-306. These results demonstrated superior effects of certain specific combinations of a specific enhancer with a specific core promoter such as CMV enhancer with hSyn promoter, particularly in use with SMN1 in an AAV vector mediated gene therapy.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed:

1. A nucleic acid comprising:
(i) a first nucleic acid region comprising a nucleic acid sequence encoding a SMN protein or variant thereof; and
(ii) a second nucleic acid region comprising one or more target segment(s) of one or more endogenous microRNA(s) (miRNA(s)),
wherein the second nucleic acid region is at 3′ of the first nucleic acid region.

2. The nucleic acid of claim 1, wherein the SMN protein or variant thereof comprises an amino acid sequence of SEQ ID NO: 33, or an amino acid sequence having at least 80%, 85%, 90%, 91%, 92%, 93%, 94%, 95%, 96%, 97%, 98%, 99% identity to SEQ ID NO: 33.

3. The nucleic acid of claim 1 or claim 2, wherein the first nucleic acid region comprises a nucleic acid sequence selected from a group consisting of SEQ ID NO: 34 and SEQ ID NO: 35.

4. The nucleic acid of any one of claims 1 to 3, wherein the second nucleic acid region comprises at least one target segment of an endogenous miRNA in heart.

5. The nucleic acid of claim 4, wherein the endogenous miRNA in heart is selected from a group consisting of hsa-mir-1-5p, hsa-mir-208a-5p, hsa-mir-208b-5p, hsa-mir-133a-1, and hsa-mir-488-5p.

6. The nucleic acid of claim 4, wherein the endogenous miRNA in heart comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 1, SEQ ID NO: 2, SEQ ID NO: 3, SEQ ID NO: 5, or SEQ ID NO: 6.

7. The nucleic acid of anyone of claims 1 to 6, wherein the second nucleic acid region comprises at least one target segment of an endogenous miRNA in liver.

8. The nucleic acid of claim 7, wherein the endogenous miRNA in liver is hsa-mir-122.

9. The nucleic acid of claim 7, wherein the endogenous miRNA in liver comprises a nucleic acid sequence at least 80%, 85%, 90%, 91%, 92%, 93%, 94%, 95%, 96%, 97%, 98%, 99% or 100% identical to SEQ ID NO: 4.

10. The nucleic acid of any one of claims 1 to 3, wherein the second nucleic acid region comprises two or more target segments of hsa-mir-133a-1.

11. The nucleic acid of claim 10, wherein the second nucleic acid region comprises at least three target segments of hsa-mir-133a-1.

12. The nucleic acid of any one of claims 1 to 3, wherein the second nucleic acid region comprises at least one target segment of hsa-mir-208a-5p, at least one target segment of hsa-mir-208b-5p, at least one target segment of hsa-mir-122, and at least one target segment of hsa-mir-133a-1.

13. The nucleic acid of claim 12, wherein the second nucleic acid region comprises 2 target segments of hsa-mir-208a-5p, 2 target segments of hsa-mir-208b-5p, 3 target segments of hsa-mir-122, and 3 target segments of hsa-mir-133a-1.

14. The nucleic acid of any one of claims 5, 8, and 10 to 13, wherein:
(i) the target segment of hsa-mir-1-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 7;
(ii) the target segment of hsa-mir-208a-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 8;
(iii) the target segment of hsa-mir-208b-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 9;
(iv) the target segment of hsa-mir-122 comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 10;
(v) the target segment of hsa-mir-133a-1 comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 11; and/or
(vi) the target segment of hsa-mir-488-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 12.

15. The nucleic acid of any one of claims 1 to 3, wherein the second nucleic acid region comprises at least 3 repeats of a nucleic acid sequence of SEQ ID NO: 11.

16. The nucleic acid of claim 15, wherein the second nucleic acid region further comprises one or more target segments of an endogenous miRNA in liver.

17. The nucleic acid of any one of claims 1 to 3, wherein the second nucleic acid region comprises:
(i) 2 repeats of a target segment having a nucleic acid sequence of SEQ ID NO: 8,
(ii) 2 repeats of a target segment having a nucleic acid sequence of SEQ ID NO: 9,
(iii) 3 repeats of a target segment having a nucleic acid sequence of SEQ ID NO: 10, and
(iv) 3 repeats of a target segment having a nucleic acid sequence of SEQ ID NO: 11.

18. The nucleic acid of any one of claims 1 to 17, wherein the second nucleic acid region further comprises one or more linkers between target segments; and wherein optionally the linker comprises 1 to 10 nucleotides.

19. The nucleic acid of claim 18, wherein the linker comprises a nucleic acid sequence selected from a group consisting of SEQ ID NO: 13, SEQ ID NO: 14, SEQ ID NO: 15, SEQ ID NO: 16, and SEQ ID NO: 17.

20. The nucleic acid of any one of claims 1 to 3, wherein the second nucleic acid region comprises:
(i) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 18;
(ii) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 19;
(iii) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 20; or
(iv) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 21.

21. The nucleic acid of any one of claims 1 to 20, wherein the first nucleic acid region further comprises:
(i) a promoter having a nucleic acid sequence of SEQ ID NO: 36 or SEQ ID NO: 37;
(ii) a promoter comprising CMV enhancer and hSyn promoter;
(iii) a promoter comprising proC3 enhancer and hSyn promoter;
(iv) a promoter comprising proA5 enhancer and hSyn promoter; or
(v) a promoter comprising proB15 enhancer and hSyn promoter,
wherein optionally the hSyn promoter comprises a nucleic acid sequence of SEQ ID NO: 38, the CMV enhancer comprises a nucleic acid sequence of SEQ ID NO: 39, the proC3 enhancer comprises a nucleic acid sequence of SEQ ID NO: 40, the proA5 enhancer comprises a nucleic acid sequence of SEQ ID NO: 41, and/or the proB15 enhancer comprises a nucleic acid sequence of SEQ ID NO: 42.

22. A vector comprising the nucleic acid of any one of claims 1 to 21.

23. The vector of claim 22, wherein the vector is a viral vector.

24. The vector of claim 23, wherein the viral vector is an adeno-associated virus (AAV) vector.

25. The vector of claim 24, wherein the AAV vector is derived from AAV1, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a combination or variant thereof.

26. The vector of claim 25, wherein the vector is a recombinant AAV9 (rAAV9) vector, or a variant thereof.

27. A recombinant AAV (rAAV) vector comprising:
(i) a first nucleic acid region comprising a transgene; and
(ii) a second nucleic acid region comprising one or more target segment(s) of one or more endogenous miRNA(s),
wherein at least one target segment is a target segment of an endogenous miRNA in heart, and at least one target segment is a target segment of an endogenous miRNA in liver;
wherein the second nucleic acid region is at 3′ of the first nucleic acid region; and
wherein the rAAV vector comprises an inverted terminal repeat (ITR) from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, or AAV44-9.

28. The rAAV vector of claim 27, wherein the first nucleic acid region comprises a nucleic acid sequence encoding a SMA protein or variant thereof comprising an amino acid sequence of SEQ ID NO: 33, or an amino acid sequence having at least 80%, 85%, 90%, 91%, 92%, 93%, 94%, 95%, 96%, 97%, 98%, 99% identity to SEQ ID NO: 33.

29. The nucleic acid of claim 27 or claim 28, wherein the first nucleic acid region comprises a nucleic acid sequence selected from a group consisting of SEQ ID NO: 34 and SEQ ID NO: 35.

30. The rAAV vector of any one of claims 27 to 29, wherein the endogenous miRNA in heart is selected from a group consisting of hsa-mir-1-5p, hsa-mir-208a-5p, hsa-mir-208b-5p, hsa-mir-133a-1, and hsa-mir-488-5p; and/or wherein the endogenous miRNA in liver is hsa-mir-122.

31. The rAAV vector of any one of claims 27 to 29, wherein:
(i) the endogenous miRNA in heart comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 1, SEQ ID NO: 2, SEQ ID NO: 3, SEQ ID NO: 5, or SEQ ID NO: 6; and/or
(ii) the endogenous miRNA in liver comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 4.

32. The rAAV vector of any one of claims 27 to 29, wherein the second nucleic acid region comprises two or more target segments of hsa-mir-133a-1.

33. The rAAV vector of claim 32, wherein the second nucleic acid region comprises at least three target segments of hsa-mir-133a-1.

34. The rAAV vector of any one of claims 27 to 29, wherein the second nucleic acid region comprises at least one target segment of hsa-mir-208a-5p, at least one target segment of hsa-mir-208b-5p, at least one target segment of hsa-mir-122, and at least one target segment of hsa-mir-133a-1.

35. The rAAV vector of any one of claims 27 to 29, wherein the second nucleic acid region comprises 2 target segments of hsa-mir-208a-5p, 2 target segments of hsa-mir-208b-5p, 3 target segments of hsa-mir-122, and 3 target segments of hsa-mir-133a-1.

36. The rAAV vector of any one of claims 30 and 32 to 35, wherein:
(i) the target segment of hsa-mir-1-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 7;
(ii) the target segment of hsa-mir-208a-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 8;
(iii) the target segment of hsa-mir-208b-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 9;
(iv) the target segment of hsa-mir-122 comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 10;
(v) the target segment of hsa-mir-133a-1 comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 11; and/or
(vi) the target segment of hsa-mir-488-5p comprises a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 12.

37. The rAAV vector of any one of claims 27 to 29, wherein the second nucleic acid region comprises at least 3 repeats of a nucleic acid sequence of SEQ ID NO: 11.

38. The rAAV vector of any one of claims 27 to 29, wherein the second nucleic acid region comprises:
(i) 2 repeats of a target segment having a nucleic acid sequence of SEQ ID NO: 8,
(ii) 2 repeats of a target segment having a nucleic acid sequence of SEQ ID NO: 9,
(iii) 3 repeats of a target segment having a nucleic acid sequence of SEQ ID NO: 10, and
(iv) 3 repeats of a target segment having a nucleic acid sequence of SEQ ID NO: 11.

39. The rAAV vector of any one of claims 27 to 38, wherein the second nucleic acid region further comprises one or more linkers between target segments, and wherein optionally the linker comprises 1 to 10 nucleotides.

40. The rAAV vector of claim 39, wherein the linker comprises a nucleic acid sequence selected from a group consisting of SEQ ID NO: 13, SEQ ID NO: 14, SEQ ID NO: 15, SEQ ID NO: 16, and SEQ ID NO: 17.

41. The rAAV vector of any one of claims 27 to 29, wherein the second nucleic acid region comprises:
(i) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 18;
(ii) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 19;
(iii) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 20; or
(iv) a nucleic acid sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 21.

42. The rAAV vector of any one of claims 27 to 41, wherein the first nucleic acid region further comprises:
(i) a promoter having a nucleic acid sequence of SEQ ID NO: 36 or SEQ ID NO: 37;
(ii) a promoter comprising CMV enhancer and hSyn promoter;
(iii) a promoter comprising proC3 enhancer and hSyn promoter;
(iv) a promoter comprising proA5 enhancer and hSyn promoter; or
(v) a promoter comprising proB15 enhancer and hSyn promoter,
wherein optionally the hSyn promoter comprises a nucleic acid sequence of SEQ ID NO: 38, the CMV enhancer comprises a nucleic acid sequence of SEQ ID NO: 39, the proC3 enhancer comprises a nucleic acid sequence of SEQ ID NO: 40, the proA5 enhancer comprises a nucleic acid sequence of SEQ ID NO: 41, and/or the proB15 enhancer comprises a nucleic acid sequence of SEQ ID NO: 42.

43. The rAAV vector of any one of claims 27 to 42, wherein the ITR is from AAV9.

44. A rAAV vector comprising a nucleic acid sequence of SEQ ID NO: 22 or SEQ ID NO: 23, SEQ ID NO: 24, or SEQ ID NO: 25, or a nucleic acid sequence having at least 80%, 85%, 90%, 91%, 92%, 93%, 94%, 95%, 96%, 97%, 98%, 99% identify to SEQ ID NO: 21, SEQ ID NO: 22, SEQ ID NO: 23, SEQ ID NO: 24 or SEQ ID NO: 25.

45. A nucleic acid comprising a nucleic acid region comprising a nucleic acid sequence encoding a SMN protein or variant thereof and a synthetic promoter comprising an enhancer and a core promoter, wherein optionally,
(i) the synthetic promoter comprises CMV enhancer and hSyn promoter;
(ii) the synthetic promoter comprises proC3 enhancer and hSyn promoter;
(iii) the synthetic promoter comprises proA5 enhancer and hSyn promoter; or
(iv) the synthetic promoter comprises proB15 enhancer and hSyn promoter.

46. The nucleic acid of claim 45, wherein the hSyn promoter comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 38.

47. The nucleic acid of claim 45, wherein the CMV enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 39.

48. The nucleic acid of claim 45, wherein the proC3 enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 40.

49. The nucleic acid of claim 45, wherein the proA5 enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 41.

50. The nucleic acid of claim 45, wherein the proB15 enhancer comprises a nucleic acid sequence selected from a group consisting of nucleic acid sequences at least 80%, 85%, 90%, 91%, 92%, 93%, 94%, 95%, 96%, 97%, 98%, 99% or 100% identical to SEQ ID NO: 42.

51. The nucleic acid of any one of claims 45 to 50, wherein the SMN protein or variant thereof comprises an amino acid sequence of SEQ ID NO: 33, or an amino acid sequence having at least 80%, 85%, 90%, 91%, 92%, 93%, 94%, 95%, 96%, 97%, 98%, 99% identity to SEQ ID NO: 33.

52. The nucleic acid of any one of claims 45 to 50, wherein the nucleic acid sequence encoding the SMN protein or variant thereof comprises a nucleic acid sequence selected from a group consisting of SEQ ID NO: 34 and SEQ ID NO: 35.

53. A vector comprising the nucleic acid of any one of claims 45 to 52.

54. The vector of claim 53, wherein the vector is a viral vector.

55. The vector of claim 54, wherein the viral vector is an adeno-associated virus (AAV) vector.

56. The vector of claim 55, wherein the AAV vector is derived from AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a combination or variant thereof.

57. The vector of claim 56, wherein the vector is a recombinant AAV9 (rAAV9) vector, or a variant thereof.

58. A rAAV vector comprising a nucleic acid sequence of SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53, or a nucleic acid sequence having at least 80%, 85%, 90%, 91%, 92%, 93%, 94%, 95%, 96%, 97%, 98%, 99% identify to SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

59. A recombinant AAV (rAAV) particle comprising
(a) the nucleic acid of any one of claims 1 to 21 and 45 to 52; or the rAAV vector of any one of claims 27 to 44 and 58; and
(b) a capsid protein of AAV1, AAV2, AAV2i8, AAV3, AAV3-B, AAV4, AAV5, AAV6, AAV7, AAV8, AAVrh8, AAVrh8R, AAV9, AAV10, AAVrh10, AAV11, AAV12, AAV13, AAV-DJ, AAV LK03, AAVrh74, AAV44-9, or a variant thereof.

60. The rAAV particle of claim 59, wherein the capsid protein is a AAV9 capsid protein or a variant thereof.

61. A pharmaceutical composition comprising the nucleic acid of any one of claims 1 to 21 and 45 to 52, the vector or rAAV vector of any one of claims 22 to 44 and 53 to 58, or the rAAV particle of any one of claims 45 to 46, 59 and 60, and a pharmaceutically acceptable excipient.

62. A method of enhancing expression of SMN protein in a cell, comprising contacting the cell with the nucleic acid of any one of claims 1 to 21 and 45 to 52, the vector or rAAV vector of any one of claims 22 to 44 and 53 to 58, the rAAV particle of any one of claims 59 to 60, or the pharmaceutical composition of claim 61.

63. A method of treating a disease or disorder in a subject, comprising administering to the subject with the nucleic acid of any one of claims 1 to 21 and 45 to 52, the vector or rAAV vector of any one of claims 22 to 44 and 53 to 58, the rAAV particle of any one of claims 59 to 60, or the pharmaceutical composition of claim 61.

64. The method of claim 63, wherein the disease or disorder is a SMN associated disease or disorder.

65. The method of claim 64, wherein the SMN associated disease or disorder is a disease or disorder associated with insufficient expression of SMN protein.

66. The method of claim 63, wherein the disease or disorder is associated with a deficient SMN protein.

67. The method of claim 63, wherein the disease or disorder is associated with a smn1 gene deletion and/or mutation.

68. The method of claim 63, wherein the disease or disorder is spinal muscular atrophy (SMA).

69. The method of claim 63, wherein the disease or disorder is SMA-I, SMA-II, SMA-III, or SMA-IV.

70. The method of any one of claims 63 to 69, wherein the subject is under 2 years old.
